SEQUENCE LISTING (33 sequences)

SEQ ID NO: 1 - protein, 440 aa, Botrytis cinerea
MQQVITQTLVDDRFIQISDSKKSEGLATDSTKRQSQEQPIHDKDPIKAATAAMAATPLVKEHQDTWYYPPDIANDLQSINLPAELKGEIFACAWEYTRCVIPNYTNWNRYVAFMRIIIMGIIAEFRGEMVDVTASNNLLGYDLDATLAALFEGTPGHKEMAREYKTFLLITADKASERRDGELFRRYVNALAQSPRHWFRMRDCDALARFTIASALACNDLDDIWFTEDQFEILTEIGDTLYDAVAFYKHRAEGETNSTFAYMPEDLRIKAYSECREILWALDAAWARNPKLANVINFVRFFGGPIHMMMRRYRFVEENLTIGKSETDKVVDQTRKNFKLWNRVDANKRSVLNTQRYKALIARSEELMFPGLAEFLEMGGDGICDKCKYRESYGAELSHQFGGVELCSECRLSWRKYLECFVERATKVFPELKTHFEVPV

SEQ ID NO: 2 - protein, 452 aa, Rutstroemia sp.
MHLRPPNNMQQVITQTLVDDRFVQLPVSQQTKSETSKGTGIDDKRWVQEQPDPAKGSNQVAAEAMKAAAPTKEHRDTWYYPPDIASDLQSVNLPAELKAEIFACAWEYTRCVIPNYTNWKRYVAFMRTIIIGVIAEFRGEMVDVTASTSILGYDLDGVLAALFEGTPGHKEMAREYKTFLLITADKASERRDGELFRRYVNALAQSPRHWFRMRDCDALARFTIASALACNDLDDIWYTEEQFEILTEIGDTLYDAVAFYKHRAEGETNSTFAYMPEDLRIKAYSECREILWALDAAWARNPKLVNVINFVRFFGGPIHMMMRRYRFVEENLTIGKSETDKVVDQTRKNFKLWNRVDANKRSIRNTQRYKALISRSEELMFPGLAEFLEVGGDGVCDKCRYRESYGAEVSHQFGGVELCSECKLSWRQYLECFVERAADVFPELKTYFEVQV

SEQ ID NO: 3 - protein, 484 aa, Colletotrichum higginsianum
MAIYEVSPEDSRAGTAEPNSIPHDQHAHKFRKFNDTWYYPSDIADDLKDIDIPDKLKAEAYACAWVYTRCVIPQYTNWDRYVAFMRIIVMGIIAEFKGSLVDVTAGDNILGYSLDGTLAALFDGTAGHELMAREYKTFLLVTADKSSGRRSGELFRRYVDSLTLSPRHWFRLRDCDALARFTIASALACNDLDDVWFTEEEFEILTEMGDTLYDAVAFYKHRAEGETNSTFAYMPEDQRVTAFARCREVLWALDVAWARRREGPSVINFTRFFGGPIHMMMRRYRFVEEGLTVGTAETLKVVNQTRRNFKLWNRVDATRVAGSSSSGSPSCNQSVQRYKDLVANRSGDLMFPELPGFLEAADKDRCGRCLYRDSYGAEGTHCFGGVQLCDDCRAGWRAYLESFPERAARVFPEILEVKPDLNNRIKNESRNDNVVIDESPQESYKLGKSSDESYVPADWKGTGKSASAAKFDSLKEISQDVLGV

SEQ ID NO: 4 - protein, 416 aa, Pseudogymnoascus sp.
MGQMQSKLGFNAGSSSNGKVTEKTLASVDIDVKTQITTPGKDSWYYPVDIANDLQGVDLPAEVKAEVLNTAWEYTRCSAPQYTNWGRYIAFMRTITICTIAEFRGKLVDVSASDNIMGYDVGSTLATLFEGTPGHTDMAREYRSFLLLTADKSSDRRDGELFRRYVNALAQSPRQWFRMRDCDALVRYTMACALVSNDHDDVWFTDEQFEILSEIYITLYDAVAFFKHRSEGETHNTYAYMPEHLRVKAYQQSREILWALDTAWAQHPGRQIAINFIRLTSGPTHLMMRRYRFVEESLTIGRRETDEVISQARTNAKLWNRVDGNKRGVNDTQRYKDLLAQSDKLMFPGLAGFLESGGDGSCKDCRYRDSYGAETSHEFGGVKLCDGCKSTWQAYLESLPERARKVLPEIVLVEVR

SEQ ID NO: 5 - protein, 443 aa, Eutypa lata
MSPKFRDTWYYPPDIANDLNGVTQLPERFKEEAYACAWEYTRCVIPQYTNWRRYVAFMRIIIMGIIAEFRGDLVDVTASDSILGYSLDGTLAELFEGTPGHALMAREYKTFLLVTADKASSRRDGELFRRYVNALAISPRAWFRMRDCDALARFTMASALACNDLDDVWPTDQQFELLAEIGDTLYDAVAFYKHRSEGETNSTFAYVPPEIRIKAFRVAREVLWALDVAYAHKPEGAPLMNFVRYFGGPIHMMMRRYRFVEEDLTVGRPETDAVVTETRRHVKLWNRVDADIHDSNGEGQTIEASSIQRYHAVLERSEELMFPELPELLERGSKPHCDRCLYRASYGAEQNHSFGGVALCRGCQAMWRGYVESLPDRTKEVFAGIVLKAPSGQEVAGPNNGIKNQQVQNGRHSNDDDFPSLPNGNAPLQNGHANGAVLVAVME

SEQ ID NO: 6 - protein, 426 aa, Leptosphaeria maculans
MLLYNSFTEGLKFTKTKILLRYYASALIDVPRDDVIEDAGVSKSQAMQNIRDRWYYPPDLANDLQDLDMPKAMKQEIFACAWEYTRCVIPQYTNWPRYVAFMRIIIIGIVAEFRGNLVDVTAGDDMMGYNLSTVLDALFLGTADRENMCREYRSFLLITADKSSERRNGELFRRYVNALAHSPRQWFRMRDADALARFTIAASLACNDLDDLKFSNMEYEILTEIGDTLYDAVAFFKHRSEGETNSTFAYMPPDMRVEAFHQAREVLWAMDVALAPKTTLQGVINFVRFFGGPIHMMMRRYRFVEEHLTIGQVETEKVVDQTRKNFKLWNRLDAKDAKAGDEKRYKDIVSNNSGEVMFPGLVEFLENEDNCPDCCFRESYGAETKHQFGGVQICSACREEWGRYMKSLPDRAVKAFPELAYVLSIP

SEQ ID NO: 7 - protein, 387 aa, Amycolatopsis mediterranei
MLMTRAEPRDNWFYPRDIEDDLKGTDLPEEIVAETLACAWEYTRCVIPQFIDWDRYIAFTRIIVIGIIAEFRGGLVDVAAGDNLLGYDLDELLDTVFAGTPGHEEMAREYRTFLLITADKSSERCNSELFRRYVNALTRSPKDWFRLRDSDALARFTIAAAMACNDITDTWFSEEEFQILTELGDTLYDAVAYYKHRAEGETNSTFAYVGHELRAESYRRCREALWALDVAWARAPAHRATINFLRYFGGPIHMMMRRYRFTEEDLTIGRPETEHVVAQTRQNIKLWHRVDVTEKSTHSVRYANVVARSEELMFPGLADMLESAADAHCDHCRHRLSYGAEPLGRFGGVELCDSCRADWATYLRTFPARAAEVFPLVGVEQAIVGLA

SEQ ID NO: 8 - protein, 377 aa, Aspergillus tubingensis
MKIQNEWYYPDDIAHDLDGIDLPKNTKDEVLACAWEYSRSVIPQYTNWERYIAFMRIIIIGVIAEFQGSIVDVTAGPKVLNYDLDEVLDELFYGTPGHLDMAREYKAFLFVTSQKVSHANSELFRRYVNALVSSPRQWFRMRDCDALARFTIASALACNDLLDIWFTDAQYDILCEIGDTMYDAVAFYKHRSEGETNSTFAYMPEDHRIDAFHRARQVLWAIDLAMAGIPGHLAVTNFLRSFGGPIHMMMRRYRFVEEDLTVGKSETEEVINQTRLNKKLWNRIDGRKTGIAEDIEHYNRSMARSDEFMFRGLADYLDRADNQHCPECIYREVYGAQRDHCFGGVQLCDQCRLEWGHFLETLPERAKRAFPDLNLRI

SEQ ID NO: 9 - protein, 432 aa, Pyrenophora tritici-repentis
MSGKTRYQELWAYPLRLYRSFGKDSTDLTSNPGSLLVHPGSITLEQSVPIVKEKWYYPTDIANDLEHIDLPQRVKEEVYACAWEYTRCVIPQYTNWKRYIAFMRIIVIGIIAEYNGELVRVAETDTLLGYNLTAVLADLFQGTRIHTEMAREFRCFLLITADKSSNWRDGLLFRSYVNALAKSPSQWFRMRDTDALIRFTLGAALACNDLDSIWYTEKQMETLCELGATLYDSVAFFKHRTEAETHSTFAYVPSSIRVQAFHQCRELLWALDVAWAGKPEHLVVINFLRFFGGPIHMMMRRYRFVEEGLTIGRPETETMVKLARQHFKLWNRVDAENCSVQDIERYKDLTARSNELMFSGLRAFLDASGEKSCEDCQQRQTYGAETSYAFGGVQLCSKCKQSWGAYVQSFPDRFNTVFPEVQKSDGTGCQIK

SEQ ID NO: 10 - protein, 416 aa, Pyrenophora teres
MPSARTSSKNGPTYDPAARKLEPKVTIQNRWYYPPDLSNDLKDIDLPTEVKGEVFACAWEYTRCVIPQYTNWPRYVAFMRIIVMGIIAEFRGSMVDVTAGDEILGYNLSEVLDALFLGTADHENMCREYRTFLLITCEKTGGKRNEELFRRYANALARSPGQWFRMRDADALARFTIAAALACNDLDDIKFSDAEYDILTEIGNTLYDAIAFFKHRSEGETNSTFSYVPSDMRVQAFHQAREVLWALDVAFAAKPGLQNVTNFVRFFGGPIHMMMRRYRFVEEGLTIGKAETDEVIGQTRQNFKLWNRLDAQDAGKKNVRRYRALVSKGRDTLMFPGLAEILESEDNCRNCTFPKSYGVVTRYEFGGVKLCASCKKNWRRYIKTLPERAVEAFPQLACVLSLEHSVGHRAYPLAAP

SEQ ID NO: 11 - protein, 404 aa, Exophiala xenobiotica
MHLEQESAESPTNGVATHEVTVVNRWYYPPDLANDMENIDMPKSMKDEVLATAWEYTRCVIPEFTNWSRYLAFARVITIGVICEFSGDMINVAESRKMLGYDVDQVLDALFKGTPGHEAMSREFRAFLLIVADKTSDRRGGELFRRYVNALARSPRQWFRMRDGDALARLTIAGLLACNDMDDVYFSDAQFEILTELADTLYDAVAFFKHRSEGETNSTFAYVPTDLRIHAFHQAREVLWALDTCWAKRPALRGVVNFIRNFGGPLHMVMRRYRFVEESLTIGRPESNKVIDQARRHVKLWNRVDAKNGGKEESSPDVQRYKHLIQNHTQDLMYTELDEFLEHSDDSTCTDCRFRRSYGADNGHIFGGVVLCNRCKVAWGAYLESFPKRTLEAFPELKGFEGFT

SEQ ID NO: 12 - protein, 406 aa, Elsinoe australis
MPKATTESHIRTYTSISHTKMLPKPTDVFYYPPDIAHDLDNLDLADQQKAEVLATSWEYTRCVIPQYTNWKRYVAFLRCIIVAVIAEYRGNVVNLTESDDILGYSVNGLIDDVFKGTAGHELMGREFKAFLLMTGEKTSERRNGELFRRYVNALSHNPQQWFRMRDCDALARFTIAGASVCNDSGNLWFNEEQFIILAELGDIMYDAVAFYKHRSEGETHSTFVYMPQDLRVKSFHVARELLWALDTAWARLPSHQIVINFVRFFGGTIHMLTRRYRFVEEDLSIGKLEDEDVVEQTRRNVKLWNRVEEKDEKFQENSSRYKEIISKHSEDLMFPGLAHALETAETGRCTDCVYRSSSGAQGVGEFGGVQLCPACREQWRQYLEALPQRVIEVFPEVLDVPGFSRS

SEQ ID NO: 13 - protein, 442 aa, Alternaria alternata
MSTFLTKVWANAIGFGIVWTGVVLFSNFFPKHDQKCAPQSDESVKNKAVVVHERWYYPPDIANDLKDIDLPDRVKQEIFATAWEYTRCVIPEYTNWSRYVAFMRIIVMGIIAEFKGSLMDVANDDNVVGYSLTEVLDTLCGGTAGHAEMCREYRCFLLMSSQKSRHQTNTMLFRRYSVALSKSPWQFFRMRDTDALARFTIGVALVCNDLDHIWFTNDQFDIMAEIGNTMYDGISYWKHRSEGEINSTFAYVPETKRVMAYHKCREALWALDVAWARQPEMKCVINFLRYFGGPIHMIMRRYRFVEEGLTLGRPEDKRVIEQTRKHVKLWNRLDEQKKTKKQETESLQQYRHVLSREKILLFNGLGEMLEKADEGLCSECSYRETYGAPQAHTFGGVVLCAAHKQGWADHTESILERMVRSFPEAAETVRISEMRATRSAAP

SEQ ID NO: 14 - protein, 419 aa, Stemphylium lycopersici
MSSFVTKVFANAIGFGIVWTGVLMFHHIRAEPVDRMSSRGDQKVAVQTNEPVKEKAVVVHERWYYPPDIANDLNGIDLPFRVKQEVFATAWEYTRCVIPEYTNWSRYVAFMRIIVMGIIAEFRGSLMDVANDDNVVGYSLTEVLNTLCGGTAGHVALSKSPWQFFRMRDTDALARFTIGTALVCNDLDHIWFSEDQFDIMAEIGNTMYDGISFWKHRSEGEINSTFAYVPEDKRVLAYHKCREALWALDVAWAKQPEMKCVINFLRYFGGPIHMIMRRYRFVEEGLTLGRPETQRVITQTRNNVKLWNRLDEQKKTKQQELESLQQYRQVLSQEKVLLFNGLGAMLEKADEDLCSKCTYRETYGAPQAHTFGGVTLCASCQQGWGAYTETVLERMARSFPEAAETIRISEMRATCLIAP

SEQ ID NO: 15 - protein, 399 aa, Fusarium oxysporum
MSNKIEHQTAVNVSNEKWYFPQELRDGLQPFNLSNETIAETLACAWEYSRCVIPNWTSWERYLAFNRTIIIAIVAEFRGDLIPEINSKNIIGYDLDELLSTLFGGTSMREDMSREFRTFLLMSAEKSRKQRDSELFGRYLNALAKSPQWWFRLRDCDALARYTIASALACNGFSEIPFREEQLQLVAELSDTLYDAVAFYKHRAEGETNSTFGYVGSEMRVENYREYREILWALDVKWAHDPAKRCVLNFIRPFGGPIHMMTRRYRFVEDGLMIGRPETDQVVELTRANFKLWHKVDSDIVKPDKDGRYMDTITKLDKLMLPGFCSLLESSARKHCPYCSNAVSSGSDIRRFGGVRLCDECKANWRSFMGAARARFAAAFPEIKNEVLKGWQCCGSSSS

SEQ ID NO: 16 - protein, 375 aa, Streptomyces sp.
MPEERWFCPDDLSNDLRDSALLPAQRAETLGQAWEYSRCTVPQFTNWGRYIALTRLCAIAVVAEVFGEHVDVLSEGPVLGYDVDDLLDTLFRDSAVREDMAREFRAAMLLSTEKSSGRRDTELMRRYVDALAHSPRDWFRLRDCDGLFRFYIAAAIACNDDDTWLTEDENRLIAEISAGLYDAVAFYKHRAEGEIHSTFAYAGAELRENAYRAYREALWSLDTRWARSTAGRCALNFIRYAGGPVHQMMRRYRFVEDGLMVGRPETDDVVSQTRRNVKLWYHIDANTAAPTDKERYDAVVRQADRLLFPGMAELLSRPDGEKCPHCRRRLSYGAEAAGEFGGVELCPACRAQWRSYLDSIPERAAHVSGISPGRH

SEQ ID NO: 17 - protein, 417 aa, Botryotinia fuckeliana
MQQVITQTLVDDRFIQISDSKKSEGLATDSTKRQSQEQPIHDKDPIKAATAAMATTPLVKEHQDTWYYPPDIANDLQSINLPAELKGEIFACAWEYTRCVIPNYTNWNRYVAFMRIIIMGIIAEFRGEMVDVTASNNLLGYDLDATLAALFEGTPGHKEMAREYKTFLLITADKASERRDGELFRRYVNALAQSPRHWFRMRDCDALARFTIASALACNDLDDIWFTEDQFEILTEIGDTLYDAVAFYKHRAEGETNSTFAYMPEDLRIKAYSECREILWALDAAWARNPKLANVINFVRFFGGPIHMMMRRYRFVEENLTIGKSETDKVVDQTRKNFKLWNRVDANKRSVLNTQRYKALIARSEELMFPGLAEFLEMGGDGICDKCKYRESTVQNCHTSLVVLNYAANADYRGEST

SEQ ID NO: 18 - DNA, 1323 bp, Botrytis cinerea
  source 1..1323: mol_type = unassigned DNA
atgcagcaggtcattacccagaccctggtggacgaccgcttcatccagatcagcgactcgaagaagtcggagggcctggccaccgactcgaccaagcgccagtcgcaggagcagccgatccatgacaaggacccgatcaaggccgcgaccgccgccatggccgccaccccgctggtgaaggagcaccaggacacctggtattatccgccggacatcgccaacgacctgcaatcgatcaacctccccgcggagctcaagggcgagatttttgcctgcgcgtgggagtacacccgctgcgtgatccccaactacaccaactggaaccgctacgtggcgttcatgcgcatcatcatcatgggcatcatcgccgagttccgcggcgagatggtggacgtgaccgcctcgaacaacctcctgggctatgacctggacgccaccctggccgccctgttcgagggcaccccgggccacaaggagatggcccgcgagtacaagaccttcctgctgatcaccgcggacaaggcctcggagcgccgcgatggcgagctgttccgccgctatgtgaacgccctggcccagtcgccgcgccattggttccgcatgcgcgactgcgacgccctcgcgcgcttcaccatcgcctcggccctggcctgcaacgacctcgacgacatctggttcaccgaggaccagttcgagatcctcaccgagatcggcgacaccctctacgacgccgtggccttctataagcatcgcgcggagggcgagacgaactcgaccttcgcgtacatgcccgaggacctccgcatcaaggcctattcggagtgccgcgagatcctgtgggccctcgacgccgcctgggcccgcaacccgaagctggccaacgtgatcaacttcgtgcgcttcttcggcggcccgatccacatgatgatgcgccgctatcgcttcgtggaggagaacctcaccatcggcaagtcggagacggacaaggtggtggaccagacccgcaagaacttcaagctctggaaccgcgtggacgccaacaagcgctcggtgctgaacacccagcgctacaaggccctcatcgcgcgctcggaggagctgatgttcccgggcctggccgagttcctcgagatgggcggcgacggcatctgcgacaagtgcaagtaccgcgagtcgtatggcgccgagctctcgcatcagttcggcggcgtggagctctgctcggagtgccggctgtcgtggcgcaagtatctcgagtgcttcgtggagcgcgcgaccaaggtgttccccgagctgaagacccatttcgaggtgccggtgtga

SEQ ID NO: 19 - protein, 440 aa, Botrytis cinerea
  REGION 1..440: note = BFV99797 from Botrytis cinerea (Seq-ID 2 of CN108753744-A)
MQQVITQTLVDDRFIQISDSKKSEGLATDSTKRQSQEQPIHDKDPIKAATAAMATTPLVKEHQDTWYYPPDIANDLQSINLPAELKGEIFACAWEYTRCVIPNYTNWNRYVAFMRIIIMGIIAEFRGEMVDVTASNNLLGYDLDATLAALFEGTPGHKEMAREYKTFLLITADKASERRDGELFRRYVNALAQSPRHWFRMRDCDALARFTIASALACNDLDDIWFTEDQFEILTEIGDTLYDAVAFYKHRAEGETNSTFAYMPEDLRIKAYSECREILWALDAAWARNPKLANVINFVRFFGGPIHMMMRRYRFVEENLTIGKSETDKVVDQTRKNFKLWNRVDANKRSVLNTQRYKALIARSEELMFPGLSEFLEMGGDGICDKCKYRESYGAELSHQFGGVELCSECRLSWRKYLECFVERATKVFPELKTHFEVPV

SEQ ID NO: 20 - protein, 440 aa, synthetic construct
  REGION 1..440: note = Protein variant 1
MQQVITNTLVDERFIQISDTKKSEGLATDSTKRQSQEQPLHDKDPIKAATAAMGATPLVKEHQDTWYYPPDIANDLQSINLPAEVKGEIFACAWEYTRCIIPNYTQWNRYVAFMRIIVMGIIAEFRGEMVDVTASNNLLGYEVDATLAALFEGTPGHKEMAREYKTFLLITADKASERKDGDLFRRYLNALAQSPMHWFRMRDCDGLARFTIASALACNDLDDIWFTEDNFEILTEIGDTLYDAVAFYKHRAEGETNSTFAYMPEDLRIKAFSECREILWALDAAWARNPKLANVINFVRFFGGPIHMMMRRYRFVEEQLTIGKSETDKVVDQTRKNFKLWNRVDANKRSLLNTQRYKALIARSEELMFPGLAEFLEMGGDGICDKCKYRESYGAELSHQFGGVDLCSECRLSWRKYLECFVERGTKVFPELKTHFEVPV

SEQ ID NO: 21 - protein, 440 aa, synthetic construct
  REGION 1..440: note = Protein variant 2
MQQIITQTLVDDRFIQISDSKKSEGLATDSTKKQSQEQPIHDRDPIKAATAAMAATPLVKEHQDTWYFPPDIANDLQSINLPAELKAEIFACAWEYTRCVIPNYTNWNRYVAFMRIIIMGIIAEFRGEMIDVTASNNLLGYDIDATLAALFEGTPGHKEMAREYKTFLLITADKASERRDGELFRKYVNALGQSPMHWFRMRDCDALARFTIASALACNDLDDIWFTEDQFEILTEIGDTIYDAVAFFKHRGEGETNSTFAYMPEDLRIKAYTECREILWAIDAAWARNPKLANVLNFVRFFGGPIHMMMRRYRFVEENLTVGKSETDKVVDQTRKQFKLWNRVDANKRSVLNTQRYKALIARSEELMFPALAEFLEMGGDGICDKCRYRESYGAELSHQFGGVELCSECRVSWRKYLECFVERATKLFPELKTHFEVPV

SEQ ID NO: 22 - protein, 440 aa, synthetic construct
  REGION 1..440: note = Protein variant 3
MQQVVSQTLVDDRFVQISDSKKSEALATDSTKRQSQEQPVHDKDPIKAASAAMAATPLVKEHQDTWWYPPEIAQDIQSIQLPAELKGELFACAWEYTRCVIPNYTNWNRYVAFMRILIMGIIAEFMGEMVDVTASNNIVGYDLDGTLAALFEGTPGHKEMAREFKTFLLITADKASERRDGELFRRYVNALAQSPRHWFRRRDCDALARFTVASALACNELDDIWFTEDQWEIVTEIGDTVYDALAFFKHRGEGETNSTFAYMPDDLRIKAYSECREVLWALDAAWARNPKLGNVINFVRFYGGPIHMMMRRYRFVEENLTVGKSETDKVIDQTRKNFKLWNRVDANKRSVLNTQRYKALIARSEELMFPGLAEYLDKGGDGICDKCKYRESYGAEVTHQFGGVEICSDCMLSWMKYIECFIERATKVFPELKTHFEVPV

SEQ ID NO: 23 - protein, 440 aa, synthetic construct
  REGION 1..440: note = Protein variant 4
MQQLITNTLLDDRFIQISDSMKSEGLGTDSTKRQSQEQPIHEKDPLRAATAAMAATPLIKEHQDTWWYPPDIANDLQSVNLPAELKGEIFACAWEYTRCVIPNYTQWNRYVAFMRIIIMALIAEFRGEKVDVTATQQLLAYDVDATLAALFEATPGHKEMAREYKTFLLITGDKASEMREGELFRKYLNALAQSPRHWFRMRDCDALARFTIASALACNDLDDIFFTEDQFEILTEIGDTLYDAVAFYKHRAEGETNSTFAYMPEDLRIKGYSECREILWALDAAYARNPKLANVINFLRWFGGPIHKMMRRYRFVEENLTIGKSETDKVVDQTRKNFKLWNRVDANKKSVLQTQRYKAVIARSDDLMFPGLAEWLDMGGDGICEKCKYRESYGAELSHNFGGVELCTECRLSWRKWLDCFVERATKVFPELKTHFEVPV

SEQ ID NO: 24 - protein, 440 aa, synthetic construct
  REGION 1..440: note = Protein variant 5
MQQVITNSVVDDKFVQLSDSKKSEGLGTDTTMRNSNEQPIHDMDPIMAATAAMAATPLVKEHQDTWYYPPDIANDLQSINLPAELKGEIFACAWEYTRCVIPNYTNWQRYVAFMRLIIMGIIAEFRGERIDITASNNLLGYDLEGTLAALFDASPGHMDMAREYKTFIVITGDKASERRDAELFMKYVNALAQSPRHWFRRRDCDGLARFTVASALACNDIDEIWFTEDQFEVLTEIADTLYDAVAFFKHRAEGETNSTFAYKPEDLRLKAYSECREVLWALDAAFARNPKLANVINFVRYYGGPIHMMMRRYRFVEENLTIGMSETDKVVDNSRKNFKLWNRIDANKRTLLNTQRYKGLIARSEELMWPGLAEFLDKGADGICEKCMYRESYGAELSHQFGGVELCTECRLSWRKYLECFVERASKLFPDLKTHYEVPL

SEQ ID NO: 25 - protein, 440 aa, synthetic construct
  REGION 1..440: note = Protein variant 6
MQQVITQTVVDDRFIQISDSKKSDGLATDSTKMQTQEQPIHDRDPIKAATAAMAATPLLKEHQDTWYYPPDIANDLQSVNVPAEVRGEIYACAWEYTRCVIPNFTNWNRYVAFMRIIVRGIIAEFMGEMIDVTASQNLLGYDIDATLAGLFEATPGHMEMGREYKTYLLITADKASERRDGELFRRYVQALAQSPRHWFRMRDCDALARFTIASALACNEVDDIFFTEENYEILTEIGDSIYDAVAFYKHRAEGETNSTFAYMPEDLRLKAYSECRELLWALDAAWARQPKVANVINFVRWFGGPVHKMMRRYRFVEENLTIGMSETDKVLDNTRMQYKLWNRIDGNKRSVVNTQRYKAVIARSEEVMFPGLAEFLEKGGEGVCDKCRYKESYGAELSHQFGGVEICSECRLSWRRYIECFVERATRLFPEVKTHYEIPL

SEQ ID NO: 26 - protein, 440 aa, synthetic construct
  REGION 1..440: note = Protein variant 7
MQQVLTQTLVDDRYVNISDSRRSEGLATDSTKRQSNEQPVHDKDPLKGATAAMAATPLVKDHQDSFYWPPDIANDLNSINVPGELKGEIFACAWEYSRCVIPNYTQWNRYLAFMRIILKGVIAEYRGEMVDLTASQNLVGYELDGTLAGVFDGTPGHMEMAREFMTYVLITADKATERRDGEIFRRYLNALAQTPRHYFRMRDCDALARFTIASALGCNDLDDIWFSEDNFEVITELGESLYDAIAFFKHRAEGETNSTFAYKPEDLRVKAYSECREVLWGLDAAWARQPKIANVVNFVRYYGGPVHMKMRRYRFVEEQLTIGKSETDKVVDQTRMNFKLWNRIDGNKRSVVNTQRYKALIARSEEVMWPGVGEFLDRGGDGICDKCMYRDSYGAELSHNFGGVELCSDCRVTWRKYLECFVDRASMLFPELKTHYEVPI

SEQ ID NO: 27 - protein, 440 aa, synthetic construct
  REGION 1..440: note = Protein variant 8
MNQIITNTIIDDKWINISDSKRSEGVGTDSTKRQSQDQPVHDRDPLKAATAAMAATPLLMEHQDTYYYPPDIANDLQSINLPAELKGEVWACAWEYTRCIIPNFTQWNRYLAFMRVIIMGIVGEFRGDRIDVSATNNLVAYELEATLAAIFEGTPGHREMAREYKTFLIITADKATDRRDGELFRRYVNGLAQSPRHWFRMRDCDALARFTIGSALACNDLDDIWWSEDNFEILTEIADTIYDAVAFYKHRAEGETNTTFAYMPDDLRIKAYTECREILWALDAGWARNPKLANLINFVRWYGGPIHRKMRRYRFVEENLSVGMTETEKVLDNTRMNFKLWNRVDGNKKSVLNTQMYMALIAKSEDLRFPGVAEFLEMGGDGICERCKYRESYGAELSHNFGGVELCTECRVSWRKYLDCFIDRASMVFPDLKTHWEVPL

SEQ ID NO: 28 - protein, 440 aa, synthetic construct
  REGION 1..440: note = Protein variant 9
MQQIVTQTIVEDRYINITDTRRSDGLATDTTMMQSQENPLHDKEPIKAATAARAATPLVKDHQDTWFWPPDIANDLQSLNIPAELKGEIFACAWEYTRCVIPNYTNWNRYIAFKRIIIKGIIAEFRGEKVDVTASNQILGYDLDATLGGIFEATPGHREMAREWKTFVLVTADKASDRRDAELFMKYIQALAQSPKHWFRMRDCDGLARYSIASALACNDVDDIWFTEDQYELLTEVADTLYDAVAFFKHRAEGETNTTFAYMPDEIRIKAYSDCRELLWALDAGWARNPMLANVVNFIRFFGGPVHRMMRRYRFVEENLTLGRSESDKLVEQTRKNWKLWNRVDGQMKSLLQSNRYRGLVAMTDDVMWPALADYLEMGGDGICEKCKYMESYGGEISHNFGGVDVCSDCMVTWKRYLDCFIERATKVFPEIKTHFDLPV

SEQ ID NO: 29 - protein, 440 aa, synthetic construct
  REGION 1..440: note = Protein variant 10
MQNVITQTLVEDMFIQITDSKKSDGLATDSTMRQTNDQPIHDKDPIRAATAAMGATPLLREHQETWYYPPEIANDIQSINVPAELKAEVFACGWEYTRCVIPNYTNWNRYVAFMRVLVRGILAEWMGEKVEVTGTNNILGYEVDATLAALFDATPAHKDMGREYKTFLLVTGEKASERKDGEVFKKYVNALGQSPMHWFRKRDCDGLARWTIASALACNDVDDIYFSEDQWEVLTEVADTLYDALAFYKHRAEGETQSTFAYMPEDLRIKAFSECREILWAVDAAWARNPMLGNLINFVRWYGGPVHKMMRRYRFLEENLTIGRSESDKLIDQTRRNFKLWQRLDGQKMTVLNTQRYRAILAMTEELMFPGIADYLERAGDGLCEMCRYRESYGAEVSHNFGGVEVCTDCRLSWKKYVECYVERASKIFPDLKTHYEVPL

SEQ ID NO: 30 - protein, 440 aa, synthetic construct
  REGION 1..440: note = Protein variant 11
MQQVVTQTLVDDMWINISDSRMSDGLGTDSTMRNTNEQPLHERDPLMGATAAKAATPLLMEHNETFFYPPDIANDLQTLNVPAEVMAEIFACAWEYTRCIIPNYSNWQRYVAFMRILVKGLLAEFRGEMIDITASNNLLGYDVDGTLAAVFEGTPGHMEMGREWKSWILLSADKGSEKRDAEIFKRYLNALAQSPMHFFRRRDCDALGRYTIGSALACNEVEDVYFTDDNYELLTELGESIYDAVAFWKHRGEGESNTTFGYKPEDVRIKAFTECRELLWAIDAAWARQPMIAQVINFVRWFGGPIHMMMRRYRFLEENLTLGRTETDKVIEQSRKNYKLWNRVDGNRKTLLNTQRYKGLVAKTDELRFPGLAEFLERGGEAICDKCKYKESYGAEITHQFGGVDICTECRVSWRRYIECFVERGTMVFPEVKTHYELPV

SEQ ID NO: 31 - protein, 440 aa, synthetic construct
  REGION 1..440: note = Protein variant 12
MNQVITQTVLDEKWIQVSDTKKSDGLATDSTRKQTNEQPVHDKEPLRAATAAKAGSPIVMEHQDSWWYPPDVGNELNTIQLPAELMAEIWACAWEYTRCIIPQYTQWNRYVAFMRIIVMAVIAEFRGERIDLSGSNQLLAYEVEASLAGVFEGTPGHKEMGREYRTFLIITADKATDKMDGELFRRYINGLGQSPRHYFRMRDCDALARFTIGSALACNELDDVWYTEDQWEIITELGETVYDAVAFYKHRAEGESQSTFGYMPEDLRIKGYSDCRDLLWAIDAGFARNPMIANLINFVRFFGGPLHKMMRRYRFVEENLTIGKTESEMIVDQSRKNWKLWNRIEAQMRSVIQTQKYKGIIAMTDEIRYPALAEFLEMGADAICDKCRYKDSYGGELTHNFGGVDLCTDCRLSWKKYIECFIERASKLFPDLKSHWEIPV

SEQ ID NO: 32 - protein, 440 aa, synthetic construct
  REGION 1..440: note = Protein variant 13
MQNVLSNTLLEDRYVQVTESRMTEGIATDSTKRNSNEQPVHDKDPLKAATAGMAASPILMEHNETYWWPPDIGNDLNTVNIPAEVKGEVYACAWEYTRCVIPNFTNWQRYLAFMRILLKGVIAEYRGEMIDVSASQNIVGYEVDGTLAGLFEGTPGHKDMGREFKTYILVSGDKASEKRDADLFRMYVQALAQSPKHYFRMRDCDALGRFSVGSALGCNELDDIFFTDDQFELVTEVAESLYDAIAFFKHRAEGESNTTFAYKPEEIRIMAYSECRELLWAVDGAYARNPMLGQVLNFVRWWGGPLHRKMRRYRFVEDNLSLGKSETDMVIDQTRKNYKLWNRLDANMMSIINSNMYKGLVAKSDDLRWPALAEFLERGGDGICDKCMWMESFGGDVSHQFGGVDVCSDCMVSWMKYIDCYIERGTMIFPEVKTHYEIPV

SEQ ID NO: 33 - protein, 440 aa, synthetic construct
  REGION 1..440: note = Protein variant 14
MQNIITQSLLEDKFVQLTESKRSDGIASDSTMKNTQEQPIHEMEPVKAGSGARGGSPVLMDHQESWFYPPEVAQDLNSVNVPADIKGELYGCAWEYSRCVIPQWTNWNRYIAFKRLILMAVIAEWMGDKLDLTASQNVLGYDIDASLAGLFDGSPAHREMAREWKSFLIVSAEKASEMMDGEVFRKYVNGLGQSPRHWFRMRDCDALGRFTVATALACNEVDDIYYSEEQFELLSELAETIYDAVAFYKHRAEGETQSTFGYRPEDLRLKGFSECREVLWALDGGWARNPMLGNILNFLRYYGGPIHKMMRRYRFVEEQLSIGMSETDKVVDNTRKNFKLWNRVEANMKSIINTQMYKALIGRTDELMFPGIGEFLEMAGDGICEKCKFRDSYGGDLTHNFGGVEICSECRITWKMFLDCWLERGTKVFPELMTHFDVPV

(19) United States
(12) Patent Application Publication
(10) Pub. No.: US 2024/0352490 A1
(43) Pub. Date: Oct. 24, 2024

(54) NOVEL PRODUCTION OF AROMA COMPOUNDS WITH IONYLIDENEETHANE SYNTHASES

(71) Applicant: BASF SE, Ludwigshafen am Rhein (DE)

(72) Inventors: Michael BREUER, Ludwigshafen am Rhein (DE); Melanie WEINGARTEN, Ludwigshafen am Rhein (DE)

(21) Appl. No.: 18/294,327

(22) PCT Filed: Aug. 1, 2022
(86) PCT No.: PCT/EP2022/071567

§ 371 Date: Feb. 1, 2024

(30) Foreign Application Priority Data
Aug. 2, 2021 (EP) 21189182.5

(51) Int. Cl. C12P 5/00; C12N 9/88

(52) U.S. Cl. CPC C12P 5/005; C12N 9/88; C12Y 402/03

(57) ABSTRACT

The present invention relates to the use of alpha-ionylideneethane as an aroma compound, and to the use of an alpha-ionylideneethane synthase in the production of one or more aroma compounds. The inventive method for preparing one or more aroma compounds comprises a) providing farnesyl diphosphate and an alpha-ionylideneethane synthase as defined herein, under conditions suitable for the alpha-ionylideneethane synthase to produce alpha-ionylideneethane, b) converting farnesyl diphosphate to alpha-ionylideneethane, in vitro or in a host cell, c) optionally, converting alpha-ionylideneethane to one or more further aroma compounds, d) isolating alpha-ionylideneethane and the optionally one or more further aroma compounds and, e) optionally, purifying alpha-ionylideneethane and the optionally one or more further aroma compounds. The invention pertains also to method for scenting a product, particularly for imparting and/or enhancing an odor or flavor, in which at least one alpha-ionylideneethane synthase is used. In addition, the invention provides an aroma compound or composition and/or fragrance composition and/or perfumed or fragranced product, comprising i) at least an alpha-ionylideneethane. Further encompassed by the invention is a perfumed or fragranced product comprising at least an alpha-ionylideneethane. The invention further relates to a method for producing alpha-ionone (4-(2,6,6-trimethylcyclohex-2-en-1-yl)but-3-en-2-one), comprising the steps in the following order: a) contacting farnesyl diphosphate with at least one alpha-ionylideneethane synthase, under conditions suitable to produce at least one alpha-ionylideneethane; b) producing the at least alpha-ionylideneethane; c) exposing the at least one alpha-ionylideneethane produced in step b) to conditions suitable for oxidative cleavage of alpha-ionylideneethane to produce alpha-ionone; and d) optionally, isolating the alpha-ionone produced in step c). The invention also relates to a host cell for producing alpha-ionone (4-(2,6,6-trimethylcyclohex-2-en-1-yl)but-3-en-2-one), wherein the host cell comprises farnesyl diphosphate and a heterologous nucleic acid encoding an alpha-ionylideneethane synthase, wherein the host cell is capable of oxidatively cleaving alpha-ionylideneethane to produce alpha-ionone. Finally, the invention relates to the use of a host cell comprising farnesyl diphosphate and a heterologous nucleic acid encoding an alpha-ionylideneethane synthase, for (i) producing alpha-ionylideneethane; (ii) producing alpha-ionone; (iii) producing vitamin A; (iv) converting alpha-ionylideneethane to alpha-ionone; (v) converting alpha-ionylideneethane to vitamin A; (vi) for heterologous reconstitution of a terpene or terpenoid; (vii) for producing an industrial product; (viii) a fermentative production system for producing a sesquiterpene.

The present invention relates to a method for preparing one or more aroma compounds, with the help of an alpha-ionylideneethane synthase and the use of such enzyme for the preparation of aroma compounds and aroma compositions and fragrances. Further the invention relates to the production of alpha-ionone with the help of this enzyme and also to the novel use of alpha-ionylideneethane (E,Z alpha-ionylideneethane=1,5,5-trimethyl-6-[(1E,3Z)-3-methyl-penta-1,3-dienyl]cyclohexene) as an aroma compound.

Specifically, the present invention relates to the use of alpha-ionylideneethane as an aroma compound, and to the use of an alpha-ionylideneethane synthase in the production of one or more aroma compounds. The inventive method for preparing one or more aroma compounds comprises, a) providing farnesyl diphosphate and an alpha-ionylideneethane synthase as defined herein, preferably an alpha-ionylideneethane as defined in claim 3, 4 or 5, under conditions suitable for the alpha-ionylideneethane synthase to produce alpha-ionylideneethane, b) converting farnesyl diphosphate to alpha-ionylideneethane, in vitro or in a host cell, c) optionally, converting alpha-ionylideneethane to one or more further aroma compounds, d) isolating alpha-ionylideneethane and the optionally one or more further aroma compounds and, e) optionally, purifying alpha-ionylideneethane and the optionally one or more further aroma compounds. The invention pertains also to a method for scenting a product, particularly for imparting and/or enhancing an odor or flavor, in which at least one alpha-ionylideneethane is used. The invention also provides an aroma compound or composition and/or fragrance composition and/or perfumed or fragranced product, comprising i) at least an alpha-ionylideneethane as defined in claim 1 or 2; ii) optionally, at least one further aroma compound different from i), and iii) optionally, at least one diluent. Further encompassed by the invention is a perfumed or fragranced product comprising at least an alpha-ionylideneethane as defined herein. The invention further relates to a method for producing alpha-ionone (4-(2,6,6-trimethylcyclohex-2-en-1-yl)but-3-en-2-one), comprising the steps in the following order: a) contacting farnesyl diphosphate with at least one alpha-ionylideneethane synthase, under conditions suitable to produce at least one alpha-ionylideneethane; b) producing the at least alpha-ionylideneethane; c) exposing the at least one alpha-ionylideneethane produced in step b) to conditions suitable for oxidative cleavage of alpha-ionylideneethane to produce alpha-ionone; and d) optionally, isolating the alpha-ionone produced in step c). The invention also relates to a host cell for producing alpha-ionone (4-(2,6,6-trimethylcyclohex-2-en-1-yl)but-3-en-2-one), wherein the host cell comprises farnesyl diphosphate and a heterologous nucleic acid encoding an alpha-ionylideneethane synthase, wherein the host cell is preferably a bacterial cell, a yeast cell, a fungal cell, an algal cell, a cyanobacterial cell, a non-human animal cell, a non-human mammalian cell, or a plant cell, and the host cell is suitable for oxidative cleavage of alpha-ionylideneethane to produce alpha-ionone. Finally, the invention relates to the use of a host cell comprising farnesyl diphosphate and a heterologous nucleic acid encoding an alpha-ionylideneethane synthase as defined in herein, for (i) producing alpha-ionylideneethane, preferably 2Z,4E-alpha-ionylideneethane (1,5,5-trimethyl-6-[(1E,3Z)-3-methyl-penta-1,3-dienyl]cyclohexene), preferably as an aroma ingredient or aroma compound, a precursor of an aroma substance or as a precursor of vitamin A; (ii) producing alpha-ionone, preferably R-alpha-ionone; (iii) producing vitamin A; (iv) converting alpha-ionylideneethane to alpha-ionone; (v) converting alpha-ionylideneethane to vitamin A; (vi) for heterologous reconstitution of a terpene or terpenoid; (vii) for producing an industrial product, preferably an aroma composition, flavour or fragrance, a pharmaceutical composition, an agricultural composition, animal feed, a human nutritional product, a cosmetic, a colorant (carotenoid) or a radical scavenger; and/or (viii) a fermentative production system for producing a sesquiterpene. Preferably, the host cell is transgenic for the nucleic acid encoding alpha-ionylideneethane synthase and comprises an active form of the alpha-ionylideneethane synthase as defined herein.

The invention pertains also to a method for preparing alpha-ionone (E-4-(2,6,6-trimethylcyclohex-2-en-1-yl)but-3-en-2-one), comprising converting alpha-ionylideneethane to alpha-ionone in the presence of farnesyl diphosphate and an alpha-ionylideneethane synthase, in vitro or in a host cell. In addition, the invention relates to a method for preparing an aroma composition, flavour, fragrance or perfume, comprising: a) Producing alpha-ionylideneethane according to the method for preparing alpha-ionylideneethane of the invention; and/or producing alpha-ionone according to the method for preparing alpha-ionone of the invention; b) isolating and, optionally, purifying alpha-ionylideneethane and/or alpha-ionone of step a); c) adding the isolated and, optionally, purified alpha-ionylideneethane and/or alpha-ionone of step b) as ingredient to an aroma chemical composition of the invention as described herein below, for example, an aroma composition, flavour, fragrance or perfume, conveying any one of the following olfactory notes: Floral-Violet or Woody-Orris (Iris) Root for alpha-ionylideneethane, and Floral-Violet for alpha-ionone. Further, the invention provides a host cell for preparing alpha-ionylideneethane and/or alpha-ionone, wherein the host cell comprises farnesyl diphosphate and a heterologous nucleic acid encoding an alpha-ionylideneethane synthase. The invention also contemplates a composition comprising (i) the host cell of the invention, alpha-ionylideneethane and/or alpha-ionone, or (ii) the alpha-ionylideneethane synthase as defined in this application, alpha-ionylideneethane and/or alpha-ionone, as well as a kit comprising the host cell of the invention, or the composition of the invention. Finally, the invention relates to the use of a) the host cell of the invention, for: (i) producing alpha-ionylideneethane, preferably 2Z,4E-alpha-ionylideneethane (1,5,5-trimethyl-6-[(1E,3Z)-3-methyl-penta-1,3-dienyl]cyclohexene), preferably as an aroma ingredient, a precursor of an aroma substance or as a precursor of vitamin A; (ii) producing alpha-ionone, preferably R-alpha-ionone; (iii) producing vitamin A; (iv) converting alpha-ionylideneethane to alpha-ionone; (v) converting alpha-ionylideneethane to vitamin A; (vi) heterologous reconstitution of a terpene or terpenoid; (vii) producing an industrial product, preferably an aroma composition, flavour or fragrance, pharmaceutical composition, an agricultural composition, animal feed, a human nutritional product, a cosmetic, a colorant (carotenoid) or a radical scavenger; (viii) a fermentative production system for producing a sesquiterpene, preferably in a host cell of the invention. The invention also relates to the use of alpha-ionylideneethane as an aroma chemical or compound.

During the past few decades, intense scientific research focused on the most abundant secondary metabolites in all living organisms, the terpenes. More than 55,000 terpenoid substances are widely distributed among different families of natural products found in all biological kingdoms.

Many terpenoids are secondary metabolites as they are commonly, not primarily, essential for growth, development, or reproduction of any organism. However, this classification does not expand on the broad additional effects of these secondary metabolites that keep an ecosystem functioning. These substances play important roles and may provide plants with evolutionary advantages in relation to their distinct chemosensory properties such as smell. Amongst others, they may exert insecticidal effects, thus protecting plants and crops against pests and pathogens, or may act as pollinator attractants in reproductive processes.

Many terpenoids are renowned for their economic importance being widely used as base structural moiety in the production of drugs, flavours, fragrances, pigments, and disinfectants. For example, alpha-ionone is used as a fragrance in perfumes, cosmetics and personal care products, as well as in household cleaners and detergents. The monoterpene alcohol linalool which is the main essential oil constituent of rosewood, Aniba rosaeodora, is among the most frequently used ingredients in perfume production. In addition, the sesquiterpene lactone, artemisinin, extracted from the shrub Artemisia annua, is used in the first-line treatment of malaria. The tricyclic diterpene taxol, isolated from the bark of the Pacific yew tree, Taxus brevifolia, and its structural analogs, are used as anticancer agents.

Terpenes are primarily synthesized in plants via common biosynthetic routes. In spite of their diverse structures and functions, all terpenes are built up of isoprene units (five-carbon atoms) following the isoprene rule. According to the number of isoprene units in their structure which are connected through head-to-tail addition, terpenes are classified according to their number of carbon atoms or sesquiterpenoid moieties, respectively: monoterpenes (C10), sesquiterpenes (C15), diterpenes (C20), triterpenes (C30), or polyterpenes having up to 30,000 connected isoprene units. Just like terpenes, terpenoids are likewise classified according to the number of isoprene units they are constituted of and are further named with the suffix “-oids”, as in monoterpenoids (C10), or in sesquiterpenoids (C15).

Isopentyl diphosphate (IPP) and its electrophilic isomer, dimethylallyl diphosphate (DMAPP), are the universal precursors in the biosynthesis of terpenes. Starting from these two building blocks, linear prenyl diphosphates are synthesized by a group of enzymes belonging to the prenyltransferases. IPP and DMAPP are condensed by the catalytic effect of the prenyltransferase geranyl diphosphate synthase to give the C10 geranyl diphosphate (GPP), the intermediate that can be converted to cyclic or linear end products, representing the group of monoterpenes.

Similarly, sesquiterpenes are generated via the addition of a third isoprene unit to GPP forming the C15 farnesyl diphosphate also known as farnesyl pyrophosphate (FPP), the biosynthetic precursor of common sesquiterpenes. Further polymerization of IPP and DMAPP produces longer prenyl diphosphates forming different classes of terpenes named according to the number of contained isoprene units.

IPP and DMAPP biosynthesis is accomplished via two independent pathways: the mevalonic acid (MVA) pathway and the 2-C-methyl-D-erythritol 4-phosphate (MEP) pathway. Although the MVA pathway was considered the universal route in the synthesis of terpenes, it was found to be less prominent in plant secondary metabolites than the MEP pathway during the last decades. MVA is dominant in most eukaryotes, archaea, few eubacteria as well as the cytosol and mitochondria of plants, and generates the precursors for sesquiterpenes (C15) and multiplied analogues such as triterpenes (C30), within the cytoplasm. On the other hand, the MEP pathway is the primary route in chloroplasts of higher plants, cyanobacteria, eubacteria, and algae. With its biosynthetic location in the plastids, MEP leads to monoterpenes (C10), diterpenes (C20) and carotenoids (C40).

The Mevalonic Acid Pathway (MVA) pathway, also known as mevalonate pathway, isoprenoid pathway, or 3-hydroxy-3-methylglutaryl-CoA (HMG-CoA) reductase pathway, was discovered in yeasts and animals in the 1950s. The MVA pathway starts with the Claisen condensation of two acetyl CoA molecules to form the acetoacetyl CoA through the catalytic action of the acetoacetyl CoA transferase enzyme. Acetoacetyl CoA is converted, via an aldol reaction with another acetyl CoA, to HMG-CoA by HMG synthase. In the next two reduction steps, two nicotinamide adenine dinucleotide phosphate molecules are required to convert HMG-CoA to mevalonic acid (MVA) with the help of HMG-CoA reductase. Subsequent phosphorylation of MVA gives mevalonate-5-diphosphate (MVAPP) via two reactions catalyzed by mevalonic acid kinase (MK) and phosphomevalonate kinase (PMK), respectively. Finally, IPP is produced from decarboxylation of MVAPP by an ATP-coupled decarboxylation reaction catalyzed by mevalonate-5-diphosphate decarboxylase (MVD). The IPP:DMAPP isomerase (IDI) then catalyzes the interconversion between IPP and DMAPP.

The Methylerythritol Phosphate Pathway (MEP), or the MVA-independent pathway, was discovered in bacteria and the chloroplasts of green algae and higher plants, in the late 1990s and early 2000s. This pathway starts with two different precursors, namely pyruvate and D-glyceraldehyde 3-phosphate (G3P). Both molecules undergo condensation catalyzed by 1-deoxy-D-xylulose 5-phosphate synthase (DXS) yielding the 1-deoxy-D-xylulose 5-phosphate (DXP), using thiamine pyrophosphate as a cofactor. In the next step, DXP is isomerized by DXP reducto-isomerase (DXR) to MEP. 4-Diphosphocytidyl-2-C-methyl-D-erythritol (CDP-ME) synthase catalyzes, consequently, the coupling between MEP and cytidine triphosphate (CTP), producing methylerythritolcytidyl diphosphate (CDP-ME). In an ATP-dependent reaction, CDP-ME kinase phosphorylates CDP-ME to 4-diphosphocytidyl-2-C-methyl-D-erythritol-2-phosphate (CDP-MEP). Subsequently, the latter undergoes cyclization to 2-C-methyl-D-erythritol-2,4-cyclodiphosphate (MEcPP), in a reaction catalyzed by MEcPP synthase, releasing cytidine monophosphate (CMP). The pathway ends up by ring opening of the cyclic pyrophosphate and the reductive dehydration of MEcPP to 4-hydroxy-3-methylbut-2-enyl-diphosphate (HMBPP) being catalyzed by HMBPP synthase. HMBPP is finally converted by HMBPP reductase to a mixture of IPP and DMAPP.

Abscisic acid (ABA) is an isoprenoid plant hormone, which is synthesized in the plastidial MEP pathway (Abscisic acid: Metabolism, transport and signalling. Da-Peng Zhan—Editor. Springer 2014). The sesquiterpenoid abscisic acid is mostly known for regulating developmental processes and abiotic stress responses in higher plants. Recent studies show that abscisic acid also exhibits a variety of pharmacological activities. However, plants are not the only organisms producing and utilising abscisic acid. For example, abscisic acid production was confirmed in phytopathogenic fungi like Botrytis cinerea, cyanobacteria, the animal parasite Toxoplasma gondii and mammals, including humans. Unlike the structurally related sesquiterpenes, which are formed from the mevalonic acid-derived precursor farnesyl diphosphate, the C15 backbone of abscisic acid is formed after cleavage of C40 carotenoids in MEP, in plants.

While abscisic acid is produced by plants through the carotenoid pathway, a small number of phytopathogenic fungi are also able to produce this sesquiterpene but they use a distinct pathway that starts with the cyclization of farnesyl diphosphate into 2Z,4E-alpha-ionylideneethane which is then subjected to several oxidation steps, by redox enzymes.

Alpha alpha-ionylideneethane and alpha-ionylideneethane synthases have been known from studies on the production of the plant hormone abscisic acid but have not been associated with a use for as aroma compounds or for the production of aroma compounds or aroma compositions, respectively.

In contrast to alpha-ionylideneethane, alpha-ionone is a known aroma compound. As mentioned, alpha-ionone is a highly valued aroma chemical conveying floral notes (Panten, J. and Surburg, H., Ullmann's Encyclopedia of Industrial Chemistry, 2000). While technical synthesis of alpha-ionone is performed, for instance, by acid catalyzed cyclization of pseudo ionone derived from condensing citral with acetone, natural alpha-ionone is generally thought to be biosynthesized by oxidative degradation of carotenoids in vivo. Accordingly, further means and methods for the synthesis of natural alpha-ionone are needed. No precedent for oxidative degradation of alpha-ionylideneethane to alpha-ionone was known before this invention.

In recent years, a number of terpenes including monoterpenes, sesquiterpenes, and their alcohols have been produced in microbial systems, in order to provide alternatives for terpenes from plant sources. Most commercially available terpenes are made by chemical synthesis, or by extraction from plant material. Plant sources are often compromised by low concentrations, harvest dependency, presence of pesticides, and/or risk of extinction of the plant species. Biotechnological production of terpenes can provide sustainable and economically viable alternatives for plant sources.

Comprising more than 30,000 compounds, terpenes are produced predominantly by plants. In light of this, further production systems for terpenes are required as alternatives for plant sources.

Recently, Otto et al. (Microb Cell Fact (2019) 18: 205) established a multistep metabolic pathway in the yeast S. cerevisiae to produce abscisic acid. In another study, the biosynthetic pathway to abscisic acid via ionylideneethane has been described in the fungus Botrytis cinerea, in a study by Inomata and co-workers (Phytochemistry. 2004 October; 65(19):2667-78. doi: 10.1016/j.phytochem.2004.08.025.). However, this pathway to abscisic acid has not been used to produce ionylideneethane and alpha-ionone, for industrial applications. Ionylideneethane has not been considered as an aroma compound so far, and the use of the abscisic acid synthesis pathway for the production of aroma compounds has not been reported by these authors. Furthermore, it was not known that ionylideneethane could also be a useful precursor for vitamin A production.

It is an object of the present invention to provide new aroma chemicals. These should have pleasant organoleptic properties. It is a further object of the present invention to provide substances which can be used as an aroma chemical in ready-to-use compositions. In particular, odor-intensive substances having a pleasant odor are sought. Furthermore, they should be combinable with other aroma chemicals, allowing the creation of novel advantageous sensory profiles. In addition, these aroma chemicals should be obtainable from readily available starting materials, allowing their fast and economic manufacturing.

Thus, the technical problem underlying the present invention may be seen as the provision of means and methods complying with the aforementioned needs. The technical problem is solved by the embodiments characterized in the claims, herein below and the Examples.

The present invention relates to a method for preparing one or more aroma compounds, comprising:
a) providing farnesyl diphosphate and an alpha-ionylideneethane synthase, under conditions suitable for the alpha-ionylideneethane synthase to produce alpha-ionylideneethane,
b) converting farnesyl diphosphate to alpha-ionylideneethane, in vitro or in a host cell,
c) optionally, converting alpha-ionylideneethane to one or more further aroma compounds,
d) isolating alpha-ionylideneethane and the optionally one or more further aroma compounds and,
e) optionally, purifying alpha-ionylideneethane and the optionally one or more further aroma compounds.

One aspect of the invention relates to a method for preparing one or more aroma compounds, comprising providing farnesyl diphosphate and an alpha-ionylideneethane synthase, converting farnesyl diphosphate to alpha-ionylideneethane, in the presence of farnesyl diphosphate and an alpha-ionylideneethane synthase, in vitro or in a host cell, optionally converting all or part of the alpha-ionylideneethane to one or more further aroma compounds, isolating alpha-ionylideneethane and the optionally one or more further aroma compounds and, optionally, purifying alpha-ionylideneethane and the optionally one or more further aroma compound.

Preferably, at least one aroma compound is alpha-ionylideneethane, more preferably, the alpha-ionylideneethane is 2Z,4E-alpha-ionylideneethane (E,Z alpha-ionylideneethane=1,5,5-trimethyl-6-[(1E,3Z)-3-methyl-penta-1,3-dienyl]cyclohexene). Preferably, at least one of the further aroma compounds is alpha-ionone, preferably R-alpha-ionone, and more preferably the method of the invention is a method for the preparation of alpha-ionylideneethane and alpha-ionone and, optionally, one or more aroma compounds other than alpha-ionylideneethane and alpha-ionone.

In a preferred embodiment of the method of the invention for preparing one or more aroma compounds, the method includes the further steps of exposing all or part of the produced at least one alpha-ionylideneethane to conditions suitable for oxidative cleavage of alpha-ionylideneethane to produce at least one alpha-ionone, preferably R-alpha-ionone, and converting all or part of the at least one alpha-ionylideneethane to alpha-ionone, preferably R-alpha-ionone, preferably by chemical or enzymatical oxidative cleavage of alpha-ionylideneethane.

The method of the invention for preparing one or more aroma compounds, such as the aroma compound alpha-ionylideneethane, can be carried out, in vitro or in a host cell. It comprises the provision of farnesyl diphosphate and one or more alpha-ionylideneethane synthase(s) as defined herein. Farnesyl diphosphate is provided as a substrate for the one or more alpha-ionylideneethane synthase(s). The method further comprises the conversion of farnesyl diphosphate to alpha-ionylideneethane by said one or more alpha-ionylideneethane synthase(s). The thus produced alpha-ionylideneethane is isolated and, optionally, purified.

Thanks to the present inventors, ionylideneethane could be identified as an aroma compound. This finding could not be expected because ionylideneethane was not considered as an aroma compound, thus far. Surprisingly, it has been found by the present inventors that alpha-ionylideneethane can be used for preparing one or more aroma compounds which convey a note of Floral-Violet and/or Woody-Orris/Iris Root to a perfume, fragrance or aroma.

In addition, the present inventors found that ionylideneethane could also be a useful precursor for vitamin A production which has not yet been reported in the prior art.

Furthermore, the present inventors advantageously found that this part of the abscisic acid synthesis pathway can be used for the industrial-scale production of aroma compounds for aroma chemical compositions of the invention which is a novel and surprising finding as well.

Alpha-ionylideneethane is a sesquiterpenoid.

A compound description of 2E,4E-alpha-Ionylideneethane can be found, e.g., in https://pubchem.ncbi.nlm.nih.gov/compound/101359914. 2Z,4E-alpha-Ionylideneethane is described, for instance, in https://pubchem.ncbi.nlm.nih.gov/compound/101760128 and https://www.biocyc.org/compound?orgid=META&id=CPD-20099.

As demonstrated in the following Examples, an alpha-ionylideneethane synthase (IES) from the phytopathogenic fungus Botrytis cinerea with the amino acid sequence depicted in SEQ ID NO. 1 was successfully cloned and expressed in Rhodobacter sphaeroides in order to produce 2Z,4E-alpha-ionylideneethane as novel aroma compound, as precursor for aroma compounds and also as potential precursor for vitamin A, by the present inventors. After scaling the production of 2Z,4E-alpha-ionylideneethane from shake flasks to DASGIP-laboratory fermenters, a novel compound was detected in the dodecane phase of the fermentation broth which could unexpectedly be identified as R-alpha-ionone. The isolation and identification of this compound is also shown in the Examples.

Hence the invention also relates to a novel method for producing alpha-ionones and mixtures of aroma compounds including alpha-ionones and/or alpha-ionylideneethane

The method of the invention for preparing one or more aroma compounds, such as the aroma compound alpha-ionylideneethane, can be performed in vitro, or in a host cell as disclosed herein. Preferably, the method for preparing the aroma compound alpha-ionylideneethane is carried out, in a host cell as defined herein.

If the method for preparing one or more aroma compounds, such as alpha-ionylideneethane, is carried out in vitro, farnesyl diphosphate is provided as a substrate in solution, e.g., in an appropriate reaction buffer. For the conversion of farnesyl diphosphate to alpha-ionylideneethane, an appropriate enzyme is used, in the in vitro method. A non-limiting example for such an enzyme is an alpha-ionylideneethane synthase (IES) which belongs to the subclass of carbon-oxygen lyases acting on phosphates (EC 4.2.3). The alpha-ionylideneethane synthase catalyzes the reaction from the substrate farnesyl diphosphate to the product alpha-ionylideneethane, possibly via a three-step reaction mechanism involving two neutral intermediates, beta-farnesene and allofarnesene, in fungi (Takino et al., BIOSCIENCE, BIOTECHNOLOGY, AND BIOCHEMISTRY 2019, VOL. 83, NO. 9, 1642-1649). Sequences of alpha-ionylideneethane synthases are disclosed elsewhere herein. Tests for determining the activity of an alpha-ionylideneethane synthase as defined herein are well known in the literature (see, e.g., Takino et al., 2019, loc. cit.). A suitable test for determining the activity of the alpha-ionylideneethane synthase as defined herein is also shown in the following Examples.

Farnesyl diphosphate (FDP), also known as farnesyl pyrophosphate (FPP), is an intermediate in both the mevalonate and non-mevalonate pathways used by organisms in the biosynthesis of terpenes, terpenoids, and sterols. A compound description of farnesyl diphosphate can be found, e.g., in https://pubchem.ncbi.nlm.nih.gov/compound/Farnesyl-diphosphate.

In plants, farnesyl diphosphate is converted to abscisic acid via oxidative cleavage of beta-carotene. Abscisic acid is one of the important phytohormones and is known as a signalling molecule for plant abiotic stress and a regulator of plant dormancy and germination. On the other hand, farnesyl diphosphate is directly cyclized to alpha-ionylideneethane which undergoes oxidation to give abscisic acid. In 2006, a putative biosynthetic gene cluster of abscisic acid was identified. Gene disruption experiments suggested two cytochrome P450s (BcABA1,2) and short-chain dehydrogenase/reductase (BcABA4) are responsible for five steps oxidative modification from alpha-ionylideneethane into abscisic acid. BcABA3 was identified as a novel terpene synthase which catalyzes a cyclization of farnesyl diphosphate to alpha-ionylideneethane and heterologous production of abscisic acid was achieved by harnessing the four bcABA genes in Aspergillus oryzae (Takino et al., 2018, J. Am. Chem. Soc., 140, 12392-12395). The BcABA3 catalyzing cyclization involves (1) ionization-initiated cyclization of farnesyl diphosphate into beta-farnesene, (2) isomerization of beta-farnesene into allofarnesene, and (3) protonation-initiated cyclization of allofarnesene to furnish alpha-ionylideneethane.

In one embodiment of the in vitro method for preparing one or more aroma compounds of the invention, farnesyl diphosphate can be converted to alpha-ionylideneethane biocatalytically, using crude protein extracts or isolated enzymes. The conversion of farnesyl diphosphate to alpha-ionylideneethane is, thereby, catalyzed by an alpha-ionylideneethane synthase as defined herein.

Appropriate conditions for carrying out the method for preparing one or more aroma compounds of the invention in vitro are described in the literature. In addition, methods for isolating and purifying of alpha-ionylideneethane, and methods for formulating said compound are described in the art; see, e.g. supporting information to the publication by Takino et al., J. Am. Chem. Soc. 2018, 140, 39, 12392-12395. A brief summary of the procedure used by the present inventors includes extraction of the fermentation broth, e.g., with tBME, and distillation of the solvent. Distillation fractions are purified by column chromatography.

As acknowledged by the skilled person, the produced alpha-ionylideneethane can also be treated chemically or subjected to one or more chemical reactions in order to obtain a desired product, such as alpha-ionone or vitamin A or precursors of vitamin A, after its isolation and/or purification.

Alternatively, the method for preparing the one or more aroma compounds of the invention can be carried out, in a host cell as defined herein. The host cell preferably produces or contains farnesyl diphosphate as a substrate. The host cell further comprises a nucleic acid encoding an enzymatically active alpha-ionylideneethane synthase for converting farnesyl diphosphate to alpha-ionylideneethane. Said nucleic acid encoding an enzymatically active alpha-ionylideneethane synthase converting farnesyl diphosphate to alpha-ionylideneethane, is preferably a heterologous nucleic acid.

According to the present invention, alpha-ionylideneethane production in a host cell may be adjusted by modifying the expression or activity of one or more proteins involved in alpha-ionylideneethane biosynthesis. It can be desirable to utilize as host cells organisms that naturally produce one or more alpha-ionylideneethane compounds. Alternatively, it can be desirable to generate production of alpha-ionylideneethane not naturally produced by the host cell.

It can be desirable to introduce one or more heterologous alpha-ionylideneethane-synthesis polypeptides into a host cell. One example for a heterologous alpha-ionylideneethane-synthesis polypeptide is an alpha-ionylideneethane synthase. As will be apparent to the skilled person, any of a variety of heterologous polypeptides as disclosed herein may be employed. Selection will consider, for instance, the particular alpha-ionylideneethane compound, e.g., E,Z-alpha-ionylideneethane, whose production is to be enhanced. The present disclosure contemplates not only introduction of heterologous alpha-ionylideneethane-synthesis polypeptides for example those depicted in SEQ ID NO: 1 to 17 and 19 to 33 and variants thereof, but also adjustment of expression or activity levels of heterologous alpha-ionylideneethane-synthesis polypeptides, including, for example, alteration of constitutive or inducible expression patterns, as explained elsewhere herein.

The produced alpha-ionylideneethane can be isolated from the host cell and purified by methods described in the art. It can then be used for the generation of a composition as disclosed herein, e.g., an aroma composition, flavour or fragrance, animal feed, a human nutritional product, a cosmetic, a colorant (carotenoid), a radical scavenger, a pharmaceutical composition or a compound for crop protection industry.

The generated alpha-ionylideneethane can also be used as a precursor for biosynthetic pathways, such as biosynthetic pathways for producing alpha-ionone, or biosynthetic pathways for producing precursors for vitamin A synthesis, in the host cell. To this end, the host cell can comprise further nucleic acids, preferably heterologous nucleic acids, encoding, for example, one, two, three, or even more, or preferably all of the enzymes of the mevalonate pathway. Such enzymes include acetyl-CoA C-acetyltransferase, hydroxymethylglutaryl-CoA synthase, (2E,6E)-farnesyl diphosphate synthase, isopentenyl-diphosphate DELTA-isomerase, hydroxymethylglutaryl-CoA reductase, diphosphomevalonate decarboxylase, mevalonate kinase, and phosphomevalonate kinase, well known in the art (see, e.g., Goldstein and Brown, Nature. 1990 Feb. 1; 343(6257):425-30. doi: 10.1038/343425a0.). The corresponding sequences of enzymes involved in the mevalonate pathway are available under, e.g., EC numbers 2.3.1.9, 2.3.3.10, 2.5.1.10, 5.3.3.2, 1.1.1.88, 4.1.1.33, 2.7.1.36, and 2.7.4.2.

Alternatively, or in addition to one, two, three, or even more, or preferably all of the enzymes of the mevalonate pathway, the host cell can comprise the nucleic acids, preferably heterologous nucleic acids, encoding, for instance, one, two, three, or even more, or preferably all of the enzymes of the deoxyxylulose phosphate (DXP or DOXP) pathway, also known as non-mevalonate pathway, mevalonate-independent pathway or MEP pathway. Such enzymes include 1-deoxy-D-xylulose-5-phosphate synthase, 1-deoxy-D-xylulose-5-phosphate reductoisomerase, 2-C-methyl-D-erythritol 4-phosphate cytidylyltransferase, 4-(cytidine 5′-diphospho)-2-C-methyl-D-erythritol kinase, 2-C-methyl-D-erythritol 2,4-cyclodiphosphate synthase, (E)-4-hydroxy-3-methylbut-2-enyl-diphosphate synthase (ferredoxin), (E)-4-hydroxy-3-methylbut-2-enyl-diphosphate synthase (flavodoxin), 4-hydroxy-3-methylbut-2-enyl diphosphate reductase, 4-hydroxy-3-methylbut-2-en-1-yl diphosphate reductase and isopentenyl-diphosphate DELTA-isomerase, described in the literature (see, e.g., Rohmer, Nat Prod Rep. 1999 October; 16(5):565-74. doi: 10.1039/a709175c).

The corresponding sequences of enzymes involved in the deoxyxylulose phosphate pathway are available under, e.g., EC numbers 2.2.1.7, 1.1.1.267, 2.7.7.60, 2.7.1.148, 4.6.1.12, 1.17.7.1, 1.17.7.3, 1.17.1.2, 1.17.7.4, and 5.3.3.2.

Alternatively, or in addition to one, two, three, or even more, or all of the enzymes of the mevalonate pathway and/or deoxyxylulose phosphate pathway, the host cell can comprise one or more nucleic acids encoding oxidative enzymes, preferably one or more nucleic acids encoding a carotene dioxygenase and/or a peroxidase that catalyse an oxidation reaction. The latter oxidative enzymes are known and described in the literature (Menzel, M. S., P., in “Flavours and Fragrances”, Berger, R. G. (ed.), Springer, Berlin, 2007, Zelena, K. et al., J. Agric. Food Chem, 2009, 57, 9951, Rajagopalan, A. et al., Adv. Synth. Catal, 2013, 355, 3321).

Host cells according to the disclosure or invention can be produced based on standard genetic and molecular biology techniques that are generally known in the art, which applies also to suitable cell culture conditions for performing said method in a host cell. In addition, methods for isolating and purifying of alpha-ionylideneethane from a host cell (see, e.g., Sambrook et al., Molecular cloning: a laboratory manual/Sambrook, Joseph; Russell, David W. —. 3rd ed. —New York: Cold Spring Harbor Laboratory, 2001; Ausubel, Current Protocols in Molecular Biology, Green Publishing Associates and Wiley Interscience, N.Y. (1994).

As appreciated by the skilled person, the produced alpha-ionylideneethane can also be treated chemically or subjected to one or more chemical reactions in order to obtain a desired product, after its isolation and/or purification from the host cell, or in the host cell.

In a preferred embodiment of the method for preparing one or more aroma compounds of the invention, in vitro or in a host cell, the alpha-ionylideneethane synthase is a fungal or bacterial alpha-ionylideneethane synthase.

As set forth in the introductory part, E,Z-alpha-ionylideneethane (1,5,5-trimethyl-6-[(1E,3Z)-3-methyl-penta-1,3-dienyl]cyclohexene; E,Z-IE, (1)) is the first cyclic intermediate of fungal abscisic acid (2) biosynthesis. The specific sesquiterpene synthase converting farnesylpyrophosphate (3) to E,Z-alpha-ionylideneethane is an alpha-ionylideneethane synthase (IE synthase); see FIG. 1.

The amino acid sequences of alpha-ionylideneethane synthases are described in the art (Takino, J. et al., J. Am. Chem. Soc., 2018, 140, 12392 FIG. S1) and available under the database accession numbers and SEQ ID numbers shown in the following Table 1. Said Table 1 further includes the organism from which the sequence comes.

TABLE 1

| | | Database |
|---|---|---|
| SEQ ID NO. | Organism | accession number |
| 1 | Botrytis cinerea/ | A0A384JQC9 |
| | Botryotinia fuckeliana | |
| 2 | Rutstroemia sp. NJR-2017a | PQE10665.1 |
| | BBW | |
| 3 | Colletotrichum higginsianum | XP_018158055.1 |
| | IMI 349063 | |
| 4 | Pseudogymnoascus sp. WSF | OBT40576.1 |
| | 3629 | |
| 5 | Eutypa lata UCREL1 | EMR65886.1 |
| 6 | Leptosphaeria maculans JN3 | XP_003843016.1 |
| | X | |
| 7 | Amycolatopsis mediterranei | AEK46506.1 |
| | S699 | |
| 8 | Aspergillus tubingensis CBS | OJI80076.1 |
| | 134.48 | |
| 9 | Pyrenophora tritici-repentis | XP_001939959.1 |
| | Pt-1C-BFP | |
| 10 | Pyrenophora teres f. teres 0-1 | EFQ89822.1 |
| 11 | Exophiala xenobiotica | XP_013319909.1 |
| 12 | Elsinoe australis | PSK60221.1 |
| 13 | Alternaria alternata | XP_018379073.1 |
| 14 | Stemphylium lycopersici | KNG44597.1 |
| 15 | Fusarium oxysporum f. sp. | XP_018257345.1 |
| | lycopersici 4287 | |
| 16 | Streptomyces sp. NRRL | WP_030303341.1 |
| | F-6131 | |
| 17 | Botrytis cinerea/ | Q14RS2 (ABA3_BOTFU) |
| | Botryotinia fuckeliana | |
| 19 | Botrytis cinerea | published as SEQ |
| | | ID NO: 2 in CN108753744 |

In addition, the inventors skillfully created synthetic alpha-ionylideneethane synthase sequences, which are shown as SEQ ID NO: 20 to 33 in the sequence listing.

Preferably, the alpha-ionylideneethane synthase comprises an amino acid sequence selected from the group consisting of:
a) an amino acid sequence as shown in SEQ ID NO. 1 to 17 or 19 to 33;
b) an amino acid sequence having at least 40% sequence identity at the amino acid level with any of SEQ ID NO. 1 to 17 or 19 to 33, having alpha-ionylideneethane synthase activity; and
c) an enzymatically active fragment of the amino acid sequence of a) or b).

Preferably, the alpha-ionylideneethane synthase comprises an amino acid sequence having at least 50%, 55%, 60%, 65%, 66%, 70%, 71%, 75%, 76%, 80%, 81%, 85%, 86%, 90%, 91%, 92%, 93%, 94%, 95%, 96%, 97%, 98%, 99% or even 100% sequence identity at the amino acid level with any of SEQ ID NO: 1 to 17 or 19 to 33, preferably with SEQ ID NO: 1 or 19, and having alpha-ionylideneethane synthase activity.

In one embodiment, the alpha-ionylideneethane synthase useful in the methods, host cells and uses of the invention has the conserved amino acids are shown by white font on black background, in FIG. 7.

In another embodiment, the alpha-ionylideneethane synthase useful in the methods, host cells and uses of the invention comprises preferably the Pfam domains DUF1175 (PF06672) and GATA (PF00320) (PFAM version 35.0); see Pfam: The protein families database in 2021: J. Mistry, S. Chuguransky, L. Williams, M. Qureshi, G. A. Salazar, E. L. L. Sonnhammer, S. C. E. Tosatto, L. Paladin, S. Raj, L. J. Richardson, R. D. Finn, A. Bateman Nucleic Acids Research (2020) doi: 10.1093/nar/gkaa913.

The alpha-ionylideneethane synthase as defined herein can be manufactured by chemical synthesis or recombinant molecular biology techniques well known to the person skilled in the art, as also shown in the following Examples. This applies mutatis mutandis to the isolation of an alpha-ionylideneethane synthase from a host cell or supernatant; see, e.g., Sambrook et al., Molecular cloning: a laboratory manual/Sambrook, Joseph; Russell, David W. —. 3rd ed. —New York: Cold Spring Harbor Laboratory, 2001; Ausubel, Current Protocols in Molecular Biology, Green Publishing Associates and Wiley Interscience, N.Y. (1994).

In a still further preferred embodiment of the method for preparing one or more aroma compounds of the invention, the host cell comprises the nucleic acid(s) encoding one, two, three, or more, or preferably all of the enzymes of the mevalonate pathway and/or the nucleic acid(s) encoding one, two, three or more, or preferably all of the enzymes of the deoxyxylulose phosphate (DOXP) pathway, for providing farnesyl diphosphate as a substrate for producing alpha-ionylideneethane.

In another aspect, it is envisaged that the host cell is fed with farnesol which is then pyrophosphorylated to provide farnesyl diphosphate/pyrophosphate, under appropriate cell culture conditions.

In another preferred embodiment of the method for preparing one or more aroma compounds of the invention, the host cell further comprises one or more nucleic acid(s) encoding oxidative enzymes, preferably one or more nucleic acids encoding a carotene dioxygenase and/or a peroxidase. Said enzymes catalyse oxidative reactions and may support, for instance, the synthesis of alpha-ionone, in the host cell, as elucidated elsewhere herein.

One potential candidate could be, for instance, a gene from Pseudocercospora pini-densiflorae CBS 125139. This organism is supposed to produce abscisic acid via alpha-ionylideneethanol, as described in Okamoto, M. et al., Phytochemistry, 1988, 27, 3465. When blasting the whole organism with the sequence of the alpha-ionylideneethane synthase from Botrytis, an 1140 bp open reading frame is found, which may be the terpene synthase mentioned in the paper from Okamato, M. et al. of 1988. So far, the present inventors do not have any experimental evidence, that the Pseudocercospora pini-densiflorae indeed transforms farnesyl diphosphate to alpha-ionylideneethanol.

The conversion of alpha-ionylideneethane to an oxidised precursor of a chemical synthesis of vitamin A is plausible using different oxidase enzymes. Reasonable candidates might by P450 monooxygenases, laccases and the like.

A direct biosynthesis of vitamin A via alpha-ionylideneethane is quite unlikely.

Preferably, the one or more aroma compounds produced by the method of the invention is or comprises alpha-ionylideneethane, more preferably the alpha-ionylideneethane is 2Z,4E-alpha-ionylideneethane (1,5,5-trimethyl-6-[(1E,3Z)-3-methyl-penta-1,3-dienyl]cyclohexene). 2Z,4E-alpha-ionylideneethane is also referred to herein as E,Z-alpha-ionylideneethane. Both terms are used interchangeably, in this disclosure.

Advantageously, the method of the invention can be used for the large-scale production of E,Z-alpha-ionylideneethane, in vitro or in a host cell, which allows for the production of, e.g., compounds with new odors, or other compositions disclosed herein.

Furthermore, E,Z-alpha-ionylideneethane can be used for synthesis of alpha-ionone or vitamin A, as disclosed herein.

The present invention pertains also to a method for preparing alpha-ionone, comprising converting alpha-ionylideneethane to alpha-ionone in the presence of farnesyl diphosphate and an alpha-ionylideneethane synthase, in vitro or in a host cell.

The present invention further provides for a method for preparing alpha-ionone, comprising the steps of a) providing farnesyl diphosphate and an alpha-ionylideneethane synthase, b) converting farnesyl diphosphate to alpha-ionylideneethane, and c) converting alpha-ionylideneethane to alpha-ionone, in vitro or in a host cell.

Also encompassed by the present invention is a method for preparing alpha-ionone or a mixture of alpha-ionylideneethane and alpha-ionone, comprising the steps of a) providing farnesyl diphosphate and an alpha-ionylideneethane synthase, b) bringing alpha-ionylideneethane in contact with an alpha-ionylideneethane synthase under conditions which allow for the production of alpha-ionone or a mixture of alpha-ionylideneethane and alpha-ionone, in vitro or in a host cell.

Specifically, the invention relates to a method for producing alpha-ionone (4-(2,6,6-trimethylcyclohex-2-en-1-yl)but-3-en-2-one), comprising the steps in the following order:
a) contacting farnesyl diphosphate with at least one alpha-ionylideneethane synthase as defined herein, preferably as defined in claims 3, 4 or 5, under conditions suitable to produce at least one alpha-ionylideneethane;
b) producing the at least alpha-ionylideneethane;
c) exposing the at least one alpha-ionylideneethane produced in step b) to conditions suitable for oxidative cleavage of alpha-ionylideneethane to produce alpha-ionone; and
d) optionally, isolating the alpha-ionone produced in step c).

In a preferred embodiment of the methods for preparing alpha-ionone of the invention, the method comprises a step of conversion of farnesyl diphosphate to alpha-ionylideneethane by an alpha-ionylideneethane synthase as disclosed herein.

In another preferred embodiment of the methods for preparing alpha-ionone of the invention, alpha-ionylideneethane is converted to alpha-ionone, preferably by oxidative cleavage. The oxidative cleavage can be carried out chemically or enzymatically.

“Oxidative cleavage” means a reaction in which a carbon-carbon bond is cleaved, with simultaneous oxidation of the carbons that had formed the carbon-carbon bond.

The oxidative cleavage to alpha-ionone can be achieved by a variety of measures known in the art for oxidation of molecules. Oxygen from the air as well as from oxygen providing substances, for example but not limited to, hydrogen peroxide or other peroxides, ozone may be used as well as enzymes providing oxygen to the reaction. As was demonstrated by the inventors, the oxidative cleavage can be done under conditions that allow for the production of alpha-ionylideneethane as well as alpha-ionone and need not be sophisticated once at least some alpha-ionylideneethane is produced.

Oxidative cleavage via enzymes can be carried out using oxidative enzymes such as a carotene dioxygenase or a peroxidase, or a combination thereof. The use of said enzymes can lead to an improved bioconversion step in the process for the production of natural alpha-ionone by a host cell disclosed herein, or in vitro.

Preferably, the alpha-ionone (4-(2,6,6-trimethylcyclohex-2-en-1-yl)but-3-en-2-one) prepared by the methods of the invention is R-alpha-ionone.

Though alpha-ionone is generally thought to be biosynthesized by oxidative degradation of carotenoids in vivo, a tentative literature search by the present inventors did not show any precedent for oxidative degradation of alpha-ionylideneethane to alpha-ionone.

Advantageously, the following Examples show a novel access to natural R-alpha-ionone, based on a microbial system. R-alpha-ionone (R-4) is probably formed by oxidative cleavage of alpha-ionylideneethane (1); see FIG. 5.

In the methods of preparing alpha-ionone of the invention, alpha-ionylideneethane is converted to alpha-ionone, thereby producing alpha-ionone. The synthesis methods can be performed in vitro, or in a host cell, preferably in a host cell of the invention.

In a preferred embodiment of the methods for preparing alpha-ionone of the invention, alpha-ionylideneethane is converted to alpha-ionone by oxidative cleavage, chemically and/or enzymatically. The conversion can be for a part of the alpha-ionylideneethane, a substantial part of it or more or less all of the alpha-ionylideneethane present. It is envisaged that the use of oxidative enzymes, e.g., carotene dioxygenase or peroxidase, can lead to an improved bioconversion step in the process for the production of natural R-alpha-ionone by a host cell disclosed herein.

Preferably, alpha-ionone (4-(2,6,6-trimethylcyclohex-2-en-1-yl)but-3-en-2-one) is R-alpha-ionone.

The present invention further relates to a host cell for producing alpha-ionone (4-(2,6,6-trimethylcyclohex-2-en-1-yl)but-3-en-2-one), wherein the host cell comprises farnesyl diphosphate and a heterologous nucleic acid encoding an alpha-ionylideneethane synthase as defined herein, preferably an alpha-ionylideneethane synthase as defined in claim 3, 4 or 5, wherein the host cell is preferably a bacterial cell, a yeast cell, a fungal cell, an algal cell, a cyanobacterial cell, a non-human animal cell, a non-human mammalian cell, or a plant cell, and the host cell is suitable for oxidative cleavage of, in one aspect capable of oxidatively cleaving alpha-ionylideneethane to produce alpha-ionone.

In a preferred embodiment,
(i) the alpha-ionylideneethane synthase as defined in claim 3, 4 or 5 converts farnesyl diphosphate to alpha-ionylideneethane, in the host cell of the invention; and/or
(ii) alpha-ionylideneethane is converted in part or total to alpha-ionone by oxidative cleavage chemically or enzymatically, in the host cell of the invention.

Alpha-ionone is a colorless to slightly yellow liquid and moderately soluble in water. Alpha-ionone occurs naturally in plants including violets, blackberries and plums. Alpha-ionone is also found in tobacco and tobacco smoke. It is a fragrant ketone responsible for the scent of Violetes. It has a sweet odor like violets and a woody, berry, floral taste. Alpha-ionone is thus used as a fragrance in perfumes, cosmetics and personal care products, as well as in household cleaners and detergents (Lalko et al., Food Chem Toxicol. 2007; 45 Suppl 1:S235-40. doi: 10.1016/j.fct.2007.09.046.). It is also utilized as a food flavoring in beverages, ice cream, baked goods and candies. Alpha-ionone is a constituent of bitter orange extract which is used widely in dietary supplements. It is used as an ingredient in cat and dog repellant applied on lawns, plants and outdoor furniture, and as a beetle attractant on roses.

According to the present invention, alpha-ionone compound production in a host cell may be adjusted by modifying the expression or activity of one or more proteins involved in alpha-ionone biosynthesis. It can be desirable to utilize as host cells organisms that naturally produce one or more ionone compounds. Alternatively, it can be desirable to generate production of alpha-ionone not naturally produced by the host cell.

It can be desirable to introduce one or more heterologous alpha-ionone-synthesis polypeptides into a host cell. As will be apparent to those of ordinary skill in the art, any of a variety of heterologous polypeptides as disclosed herein may be employed. To provide an example, farnesyl diphosphate can be converted to alpha-ionylideneethane by an alpha-ionylideneethane synthase as disclosed herein, followed by conversion of alpha-ionylideneethane to alpha-ionone by an enzyme catalysing oxidative cleavage, such as a carotene dioxygenase or peroxidase, as disclosed herein. Selection will consider, for instance, the particular ionone compound, such as alpha-ionone or R-alpha-ionone, whose production is to be enhanced. The present disclosure contemplates not only introduction of heterologous alpha-ionone-synthesis polypeptides, but also adjustment of expression or activity levels of heterologous alpha-ionone-synthesis polypeptides, including, for example, alteration of constitutive or inducible expression patterns, as explained elsewhere herein.

At one time, the extraction of fragrant compounds from flowers and other plants was the sole source of materials for products such as perfumes. For instance, bio-degradation of carotenoids has been shown to be an important route for apocarotenoids formation, in the recent years. However, it is now more economical to synthesize these compounds in the laboratory. Advantageously, the methods of the invention can be used for the large-scale production of alpha-ionone, in vitro or in a host cell, which allows for the production of, e.g., compounds with new odors, or other compositions disclosed herein.

For determination of the content of the alpha-ionylideneethane and/or alpha-ionone produced by the methods of the invention, several instrumental techniques, such as gas chromatography-flame ionization detector (GC-FID), gas chromatography-mass selective detector (GC-MSD), high performance liquid chromatography-evaporative light scattering detector (HPLC-ELSD), high performance liquid chromatography-refractive index detector (HPLC-RID), high performance liquid chromatography-variable wavelength detector (HPLC-VWD), gel permeation chromatography (GPC), high performance thin layer chromatography (HPTLC), nuclear magnetic resonance (NMR), termogravimetric analysis (TGA), and infrared spectroscopy (IR) can be used which are known in the art; see, e.g., review by Jiang et al, Curr Protoc Plant Biol. 2016; 1: 345-358. doi:10.1002/cppb.20024. Further methods for extraction, purification and analysis of alpha-ionylideneethane and/or alpha-ionone are shown in the Examples.

In a preferred embodiment, alpha-ionylideneethane(s) is (are) produced in a ratio to alpha-ionone of about 8:1 or less, preferably about 5:1, 4:1, 3:1, 2:1, 1:1, or 0.5:1 or even 0.1:1, in the methods of the invention.

In another preferred embodiment, at least 10%, preferably at least 20%, 30%, 40%, 50%, 70%, 80%, 90%, 95% or 99% of the alpha-ionylideneethane(s) is (are) converted to alpha-ionone, in the methods of the invention.

The invention further relates to the use of alpha-ionylideneethane as an aroma compound.

Preferably, alpha-ionylideneethane has a note of Floral-Violet and/or Woody-Orris/Iris Root.

The invention also pertains to the use of an alpha-ionylideneethane synthase in the production of one or more aroma compounds.

Preferably, the alpha-ionylideneethane synthase is selected from the group consisting of:
a) the alpha-ionylideneethane synthase belongs to the subclass of carbon-oxygen lyases acting on phosphates (EC 4.2.3); and
b) the alpha-ionylideneethane synthase is a fungal or bacterial alpha-ionylideneethane synthase; and
c) the alpha-ionylideneethane synthase comprises an amino acid sequence selected from the group consisting of:
   i) an amino acid sequence as shown in any of SEQ ID NO. 1 to 17 or 19 to 33;
   ii) an amino acid sequence having at least 40% sequence identity at the amino acid level with any of SEQ ID NO. 1 to 17 or 19 to 33, having alpha-ionylideneethane synthase activity; and
   iii) an enzymatically active fragment of the amino acid sequence of a) or b), having alpha-ionylideneethane synthase activity; and
d) any combination of a) to c) above.

In one embodiment, the alpha-ionylideneethane synthase is for preparing one or more aroma compounds which convey a note of Floral-Violet and/or Woody-Orris/Iris Root to a perfume, fragrance or aroma.

In another embodiment, the alpha-ionylideneethane is produced by an alpha-ionylideneethane synthase as disclosed herein, preferably an alpha-ionylideneethane synthase as defined in claim 3, 4 or 5.

The definitions, explanations and embodiments with respect to the methods of the invention apply mutatis mutandis to the uses of the invention.

The present invention further pertains to a method for preparing vitamin A, comprising converting alpha-ionylideneethane to vitamin A, preferably in vitro or in a host cell, the method comprising converting alpha-ionylideneethane chemically or enzymatically via the respective alcohol to (2E,4E)-3-methyl-5-(2,6,6-trimethylcyclohex-2-en-1-yl)penta-2,4-dien-1-ol, followed by Wittig salt formation under isomerisation ([(2E,4E)-3-methyl-5-(2,6,6-trimethylcyclohexen-1-yl)penta-2,4-dienyl]-triphenyl-phosphonium), and Wittig reaction with C5-aldehyde [(E)-3-methyl-4-oxo-but-2-enyl] acetate, thereby preparing vitamin A; see also FIG. 6.

Specifically, the invention relates to a method for preparing vitamin A, the method comprising the steps of:
a) contacting farnesyl diphosphate with one or more alpha-ionylideneethane synthases as defined herein, preferably with one ore more alpha-ionylideneethane synthases as defined in claim 3, 4 or 5, under conditions suitable to produce at least one alpha-ionylideneethane,
b) producing alpha-ionylideneethane,
c) converting the alpha-ionylideneethane chemically or enzymatically, via the respective alcohol to (2E,4E)-3-methyl-5-(2,6,6-trimethylcyclohex-2-en-1-yl)penta-2,4-dien-1-ol, followed by Wittig salt formation under isomerisation ([(2E,4E)-3-methyl-5-(2,6,6-trimethylcyclohexen-1-yl)penta-2,4-dienyl]-triphenyl-phosphonium), and Wittig reaction with C5-aldehyde [(E)-3-methyl-4-oxo-but-2-enyl] acetate, thereby preparing vitamin A.

Preferably, at least one, more preferably two, even more preferably all of the method steps of the methods for preparing vitamin A of the invention is (are) performed in vitro. In another embodiment of this method of the invention, the method comprises using a host cell comprising farnesyl diphosphate and a heterologous nucleic acid encoding an alpha-ionylideneethane synthase as defined herein, preferably an alpha-ionylideneethane synthase as defined in claim 3, 4 or 5. Preferably, the host cell is a bacterial cell, a yeast cell, a fungal cell, an algal cell, a cyanobacterial cell, a non-human animal cell, a non-human mammalian cell, or a plant cell, more preferably a bacterial cell or a yeast cell. Preferably, the host cell is used in fermentation.

The synthesis of vitamin A and/or similar carotenoids is may not be by biocatalysis/enzymes alone. Possible is also a chemo-enzymatic conversion: Glucose via farnesyl diphosphate to form alpha-ionylideneethane or the respective alcohol (alpha-ionylideneethanol) would make up the bio-part, followed by a purely chemo-catalytic conversion of alpha-ionylideneethane/alpha-ionylideneethanol to vitamin A.

E,Z-alpha-ionylideneethane is potentially an interesting precursor for vitamin A even though the position of the cyclohexene double bond in E,Z-alpha-ionylideneethane is different than in vitamin A.

One goal of this invention was to show the proof of the concept of alpha-ionylideneethane synthesis in Rhodobacter. In order to assess whether alpha-ionylideneethane is a reasonable starting point for a hybrid bio-chemical synthesis of vitamin A, sufficient starting material can now be obtained for example but not limited to by fermentation of the newly constructed Rhodobacter strain ROB034, described in the following Examples.

In particular, the functionalization of the terminal methyl group on C-11 of alpha-ionylideneethane to accommodate carbon-chain elongation and shifting the cyclohexene double bond from C-5 to C-4 is an important milestone, in this context.

Moreover, it should also be possible to provide material for assessing the chemical or biocatalytic conversion of E,Z-alpha-ionylideneethane to more direct precursors of vitamin A, according to the present invention.

Methods for extraction and determination of vitamin A are well described in the art; see, e.g., review by Zhang et al., Molecules. 2018 June; 23(6): 1484. Published online 2018 Jun. 19. doi: 10.3390/molecules23061484.

The invention further relates to a method for scenting a product, particularly for imparting and/or enhancing an odor or flavor, in which at least one alpha-ionylideneethane as defined herein, preferably an alpha-ionylideneethane having a note of Floral-Violet and/or Woody-Orris/Iris Root, more preferably 2Z,4E-alpha-ionylideneethane., is used.

The invention further relates to a method for scenting a product, particularly for imparting and/or enhancing an odor or flavor, in which at least one alpha-ionylideneethane synthase as defined herein is used, wherein the method includes the step of. preparing one or more aroma compounds according to the methods of the invention, followed optionally by a step of purification of the one or more aroma compounds and a subsequent step of scenting a product with the one or more aroma compounds.

A further aspect of the present invention relates to a method of modifying the aroma of a ready-to-use composition. Said method comprises the step of incorporating the alpha-ionylideneethane and/or alpha-ionone, the latter preferably produced by the methods of the present invention, into a ready-to-use composition so as to obtain an aroma-modified ready-to-use composition.

The compound of the present invention and aroma chemical compositions thereof possess advantageous organoleptic properties, in particular a pleasant aroma. Therefore, they can be favorably used as aromatizing ingredients in perfume compositions, body care compositions (including cosmetic compositions and products for oral and dental hygiene), hygiene articles, cleaning compositions (including dishwashing compositions), textile detergent compositions, compositions for scent dispensers, foods, food supplements, pharmaceutical compositions, crop protection compositions and other ready-to-use compositions.

The pleasant aroma, low volatility and excellent solubility make the alpha-ionylideneethane and/or alpha-ionone, the latter preferably produced by the methods of the invention, a suitable ingredient in compositions where a pleasing aroma is desirable. By virtue of its physical properties, the alpha-ionylideneethane and/or alpha-ionone is well combinable with other aroma chemicals and customary ingredients in aromatized ready-to-use compositions such as, in particular, perfume compositions. This allows, e.g., the creation of aroma compositions, in particular perfume compositions having novel advantageous sensory profiles.

Furthermore, the alpha-ionylideneethane and/or alpha-ionone produced by the methods of the invention can be produced in good yields and purities by a simple synthesis starting from readily available starting materials. Thus, the alpha-ionylideneethane and/or alpha-ionone produced by the methods of the invention can be produced in large scales and in a simple and cost-efficient manner.

In addition, the present invention provides a method for preparing an aroma chemical composition of the invention such as but not limited to an aroma composition, flavour, fragrance or perfume, comprising:
a) Producing alpha-ionylideneethane according to the method for preparing alpha-ionylideneethane of the invention;
b) isolating and, optionally, purifying alpha-ionylideneethane of step a);
c) adding the isolated and, optionally, purified alpha-ionylideneethane of step b) as ingredient an aroma chemical composition of the invention as described herein, for example, an aroma composition, flavour, fragrance or perfume, conveying any one of the following olfactory notes: Floral-Violet and/or Woody-Orris (Iris) Root for alpha-ionylideneethane, and Floral-Violet for alpha-ionone.

Orris root (Rhizoma iridis) is the root of Iris germanica and Iris pallida and Iris florentina. The most valued component of orris root is oil of orris (0.1-0.2%), a yellow-white mass containing myristic acid. Once important in western herbal medicine, it is now used mainly as a fixative and base note in perfumery; see, e.g., John Charles Sawer, Odorographia a natural history of raw materials and drugs used in the perfume industry intended to serve growers, manufacturers and consumers. The odor profile of orris root is a powdery earthy rooty scent, with woody, violet flower nuances. The expressions Woody-Orris (Iris) Root or Woody-Orris/Iris Root are to be understood to refer to the typical note of these orris root or Iris root.

Further, the present invention provides a method for preparing an aroma composition, flavour, fragrance or perfume, comprising:
a) Producing alpha-ionone according to any one of the methods for preparing alpha-ionone of the invention;
b) isolating and, optionally, purifying alpha-ionone of step a);
c) adding the isolated and, optionally, purified alpha-ionone of step b) as ingredient aroma chemical composition of the invention such as but not limited to an aroma composition, flavour, fragrance or perfume, conveying any one of the following olfactory notes: Floral-Violet or Woody-Orris (Iris) Root for alpha-ionylideneethane, and Floral-Violet for alpha-ionone.

As acknowledged by the skilled person, the latter method can also include the production, isolation and optional purification of alpha-ionylideneethane as additional method steps.

Accordingly, the present invention also contemplates a method for preparing an aroma composition, flavour, fragrance or perfume, comprising:
a) Producing alpha-ionylideneethane according to the method for preparing alpha-ionylideneethane of the invention; and/or
b) producing alpha-ionone according to any one of the methods for preparing alpha-ionone of the invention;
c) isolating and, optionally, purifying alpha-ionylideneethane of step a) and/or alpha-ionone of step b);
d) adding the isolated and, optionally, purified alpha-ionylideneethane and/or alpha-ionone of step c) as ingredient to an aroma chemical composition of the invention such as but not limited to an aroma composition, flavour, fragrance or perfume, conveying any one of the following olfactory notes: Floral-Violet or Woody-Orris (Iris) Root for alpha-ionylideneethane, and Floral-Violet for alpha-ionone.

Monoterpenes and sesquiterpenes are industrially used as flavour, fragrant, and cosmetic constituents. Fragrances and aromas are used as essential additives enhancing the final quality of foods and beverages, as well as in body care and other hygienic products. In recent time, there is a raising demand, however, for products of natural origin. Therefore, natural flavour compounds that can improve the sensory appeal of these products gained larger value and became more expensive than their artificial counterparts. The alpha-ionylideneethane and/or alpha-ionone, the latter preferably produced by the methods of the invention, can advantageously be used for generating an aroma composition, flavour, fragrance or perfume or any other products disclosed herein.

Means and methods for preparing an aroma compound or composition, flavour, fragrance or perfume are well known in the art; see, e.g., Flavours and Fragrances: Chemistry, Bioprocessing and Sustainability R G Berger; Black et al., EP2897465B1, Chromatography of Aroma Compounds and Fragrances, Cserhati, T. (2010).

The alpha-ionylideneethane and/or alpha-ionone, the latter preferably produced by the methods of the invention, can generally be used in a ready-to-use composition, in particular in an aromatized ready-to-use composition. “Aromatized ready-to-use composition”, as used herein, refers to a ready-to-use composition which predominately induces a pleasant odor and/or taste impression. In preferred embodiments, the aromatized ready-to-use composition is a scented ready-to-use composition, i.e. induces a pleasant odor. Scented ready-to-use compositions are, for example, compositions used in personal care, in home care, in industrial applications as well as compositions used in other applications, such as pharmaceutical compositions or crop protection compositions.

Preferably, the alpha-ionylideneethane and/or alpha-ionone the latter preferably produced by the methods of the invention, is used in a composition selected from the group consisting of perfume compositions, body care compositions (including cosmetic compositions and products for oral and dental hygiene), hygiene articles, cleaning compositions (including dishwashing compositions), textile detergent compositions, compositions for scent dispensers, foods, food supplements, pharmaceutical compositions and crop protection compositions. The alpha-ionylideneethane and/or alpha-ionone the latter preferably produced by the methods of the invention, is used as an aroma chemical, preferably as a fragrance, in the above compositions.

In particular, the alpha-ionylideneethane and/or alpha-ionone is used to impart a note that is reminiscent of sweet, floral, violet, orris, rooty and/or woody; or is used to produce a scent that is reminiscent of, floral and/or woody elements to the compositions.

Details to the above-listed compositions are given below. Similarly, the alpha-ionylideneethane and/or alpha-ionone the latter preferably produced by the methods of the invention, can improve the sensory profiles of aroma chemical compositions as a result of synergistic effects with other aroma chemical (e.g., other fragrances) comprised in the compositions, which means that the compound can provide a booster effect for said other aroma chemicals. The compound is, therefore, suitable as a booster for other aroma chemicals.

Accordingly, the invention also relates to the use of the alpha-ionylideneethane alone or in combination with alpha-ionone for modifying the aroma character (e.g., the scent character) of an aromatized (e.g., fragranced) composition; and specifically to the use as a booster for other aroma chemicals.

Booster effect of a substance means that the substance enhances and intensifies in aroma chemical formulations (such as, e.g., perfumery formulations) the overall sensory (e.g., olfactory) impression of the formulation. In the mint range, for example, it is known that menthyl methyl ether intensifies the perfumery or taste mixtures of peppermint oils and particularly in top notes brings about a considerably more intensive and more complex perception although the ether itself, being a pure substance, develops no particular intensive odor at all. In fragrance applications, Hedione® (methyl dihydrojasmonate), which as a pure substance only exhibits a light floral jasmine note, reinforces diffusion, freshness and volume of a perfume composition as an odor booster. Booster effects are particularly desired when top-note-characterized applications are required, in which the odor impression is to be conveyed particularly quickly and intensively, for example, in deodorants, air fresheners or in the taste sector in chewing gums.

To achieve such a booster effect, alpha-ionylideneethane and/or alpha-ionone can be used, for example, in an amount of 0.001 to 10 wt. % (weight-%), such as in an amount of 0.01 to 2 wt. %, preferably from 0.05 to 1 wt. %, in particular in an amount of from 0.1 to 0.5 wt. %, based on the total weight of the resulting aroma chemical composition.

Furthermore, the alpha-ionylideneethane alone or in combination with alpha-ionone can have further positive effects on the composition in which it is used. For example, the compound can enhance the overall performance of the composition into which it is incorporated, such as the stability, e.g. the formulation stability, the extendibility or the staying power of the composition.

In an embodiment, the present invention relates to an aroma chemical composition comprising the alpha-ionylideneethane without or with alpha-ionone and:
(i) at least one additional aroma chemical, or
(ii) at least one non-aroma chemical carrier, or
(iii) both of (i) and (ii).

The term “aroma composition” or “aroma chemical composition”, as used herein, refers to a composition which induces a pleasant aroma, e.g., a pleasant odor impression. Both terms are used interchangeably, if not indicated otherwise.

The non-aroma chemical carrier in the aroma chemical composition of the invention can be, in particular, selected from surfactants, oil components and solvents.

The additional aroma chemical in one aspect is different from alpha-ionylideneethane or alpha-ionone, i.e. is neither a stereoisomer of alpha-ionylideneethane or alpha-ionone or a mixture of two or more stereoisomers of alpha-ionylideneethane or alpha-ionone.

By virtue of its physical properties, alpha-ionylideneethane and/or alpha-ionone produced by the methods of the invention is well combinable with other aroma chemicals (e.g., other fragrances) and other customary ingredients in aromatized (e.g., fragranced) ready-to-use compositions such as, in particular, perfume compositions. This allows, e.g., the creation of aroma compositions (e.g., perfume compositions) which have novel advantageous sensory profiles. Especially, as already explained above, the compound can provide a booster effect for other aroma chemicals (such as other fragrances).

Accordingly, in one preferred embodiment, the aroma chemical composition comprises a alpha-ionylideneethane without or with alpha-ionone as defined herein; and at least one additional aroma chemical that is different from alpha-ionylideneethane or alpha-ionone.

The additional aroma chemical can, for example, be one, preferably 2, 3, 4, 5, 6, 7, 8 or further aroma chemicals, selected from the group consisting of:

geranyl acetate, alpha-hexylcinnamaldehyde, 2 phenoxyethyl isobutyrate, dihydromyrcenol, methyl dihydrojasmonate, 4,6,6,7,8,8 hexamethyl-1,3,4,6,7,8-hexa-hydro-cyclopenta[g]benzopyran, tetrahydrolinalool, ethyllinalool, benzyl salicylate, 2 methyl-3-(4-tert-butylphenyl)propanal, cinnamyl alcohol, 4,7 methano-3a,4,5,6,7,7a-hexahydro-5 indenyl acetate and/or 4,7 methano-3a,4,5,6,7,7a-hexahydro-6-indenyl acetate, citronellol, citronellyl acetate, tetrahydrogeraniol, vanillin, linalyl acetate, styrolyl acetate, octahydro-2,3,8,8-tetramethyl-2-acetonaphthone and/or 2 acetyl-1,2,3,4,6,7,8-octahydro-2,3,8,8-tetramethylnaphthalene, hexyl salicylate, 4 tert-butylcyclohexyl acetate, 2-tert-butylcyclohexyl acetate, alpha-ionone, n alpha-methylionone, alpha-isomethylionone, coumarin, terpinyl acetate, 2 phenylethyl alcohol, 4-(4-hydroxy-4-methylpentyl)-3-cyclohexene-carboxaldehyde, alpha-amylcinnamaldehyde, ethylene brassylate, (E) and/or (Z)-3-methylcyclopentadec-5 enone, 15-pentadec-11-enolide and/or 15-pentadec-12-enolide, 15-cyclopentadecanolide, 1-(5,6,7,8-tetrahydro-3,5,5,6,8,8-hexamethyl-2-naphthalenyl)ethanone, 2-isobutyl-4-methyltetrahydro-2H pyran-4-ol, 2-ethyl-4-(2,2,3-trimethyl-3-cyclopenten-1-yl)-2-buten-1-ol, cis-3-hexenyl acetate, trans-3-hexenyl acetate, trans-2/cis-6-nonadienol, 2,4-dimethyl-3-cyclohexenecarboxaldehyde, 2,4,4,7-tetramethyloct-6-en-3-one, 2,6-dimethyl-5-hepten-1-al, borneol, 3 (3 isopropylphenyl)butanal, 2-methyl-3-(3,4-methylenedioxyphenyl)-propanal, 3-(4-ethylphenyl)-2,2-dimethylpropanal, 7-methyl-2H 1,5-benzodioxepin-3(4H)-one, 3,3,5-trimethylcyclohexyl acetate, 2,5,5 trimethyl-1,2,3,4,4a,5,6,7-octahydronaphthalen-2-ol, 3-(4-tert-butylphenyl)-propanal, ethyl 2-methylpentanoate, ethoxy methoxy cyclododecane, 2,4-dimethyl-4,4a,5,9b-tetrahydroindeno[1,2-d][1,3]dioxine, (2-tert-butylcyclohexyl) acetate and 3-[5,5,6-trimethylbicyclo[2.2.1]hept-2-yl]cyclohexan-1-ol, 2,4-diethylocta-2,6-dienal.

In yet another preferred embodiment, the at least one aroma chemical (i) is selected from the group consisting of methyl benzoate, benzyl acetate, geranyl acetate, 2-isobutyl-4-methyltetrahydro-2H-pyran-4-ol, linalool, 2-isobutyl-4-methyltetrahydro-2H-pyran-4-ol and methyl benzoate.

In yet another preferred embodiment, the at least one aroma chemical (i) is selected from the group consisting of ethylvanillin, vanillin, 2,5-dimethyl-4-hydroxy-2H-furan-3-one (furaneol) or 3-hydroxy-2-methyl-4H-pyran-4-one (maltol).

Further aroma chemicals with which the compound of formula (1) and/or (4) can be combined to give a composition according to the presently claimed invention can be found, e.g., in S. Arctander, Perfume and Flavor Chemicals, Vol. I and II, Montclair, N. J., 1969, self-published or K. Bauer, D. Garbe and H. Surburg, Common Fragrance and Flavor Materials, 4th Ed., Wiley-VCH, Weinheim 2001. Specifically, mention may be made of: extracts from natural raw materials such as essential oils, concretes, absolutes, resins, resinoids, balsams, tinctures such as, e.g., ambergris tincture; amyris oil; angelica seed oil; angelica root oil; aniseed oil; valerian oil; basil oil; tree moss absolute; bay oil; mugwort oil; benzoin resin; bergamot oil; beeswax absolute; birch tar oil; bitter almond oil; savory oil; buchu leaf oil; cabreuva oil; cade oil; calmus oil; camphor oil; cananga oil; cardamom oil; cascarilla oil; cassia oil; cassia absolute; castoreum absolute; cedar leaf oil; cedar wood oil; cistus oil; citronella oil; lemon oil; copaiba balsam; copaiba balsam oil; coriander oil; costus root oil; cumin oil; cypress oil; davana oil; dill weed oil; dill seed oil; Eau de brouts absolute; oak moss absolute; elemi oil; tarragon oil; eucalyptus citriodora oil; eucalyptus oil; fennel oil; pine needle oil; galbanum oil; galbanum resin; geranium oil; grapefruit oil; guaiacwood oil; gurjun balsam; gurjun balsam oil; helichrysum absolute; helichrysum oil; ginger oil; iris root absolute; iris root oil; jasmine absolute; calmus oil; camomile oil blue; roman camomile oil; carrot seed oil; cascarilla oil; pine needle oil; spearmint oil; caraway oil; labdanum oil; labdanum absolute; labdanum resin; lavandin absolute; lavandin oil; lavender absolute; lavender oil; lemongrass oil; lovage oil; lime oil distilled; lime oil pressed; linalool oil; Litsea cubeba oil; laurel leaf oil; mace oil; marjoram oil; mandarin oil; massoia bark oil; mimosa absolute; musk seed oil; musk tincture; clary sage oil; nutmeg oil; myrrh absolute; myrrh oil; myrtle oil; clove leaf oil; clove flower oil; neroli oil; olibanum absolute; olibanum oil; opopanax oil; orange blossom absolute; orange oil; origanum oil; palmarosa oil; patchouli oil; perilla oil; peru balsam oil; parsley leaf oil; parsley seed oil; petitgrain oil; peppermint oil; pepper oil; pimento oil; pine oil; pennyroyal oil; rose absolute; rose wood oil; rose oil; rosemary oil; Dalmatian sage oil; Spanish sage oil; sandalwood oil; celery seed oil; spike-lavender oil; star anise oil; styrax oil; tagetes oil; fir needle oil; tea tree oil; turpentine oil; thyme oil; tolubalsam; tonka absolute; tuberose absolute; vanilla extract; violet leaf absolute; verbena oil; vetiver oil; juniper berry oil; wine lees oil; wormwood oil; winter green oil; hyssop oil; civet absolute; cinnamon leaf oil; cinnamon bark oil, and fractions thereof, or ingredients isolated therefrom;
individual fragrances from the group of hydrocarbons, such as e.g. 3 carene; alpha-pinene; beta-pinene; alpha-terpinene; gamma-terpinene; p-cymene; bisabolene; camphene; caryophyllene; cedrene; farnesene; limonene; longifolene; myrcene; ocimene; valencene; (E,Z)-1,3,5-undecatriene; styrene; diphenylmethane;
is the aliphatic alcohols such as e.g. hexanol; octanol; 3-octanol; 2,6-dimethylheptanol; 2-methyl-2-heptanol; 2-methyl-2-octanol; (E)-2-hexenol; (E)- and (Z)-3-hexenol; 1 octen-3-ol; mixture of 3,4,5,6,6-pentamethyl-3/4-hepten-2-ol and 3,5,6,6-tetramethyl-4-methyleneheptan-2-ol; (E,Z)-2,6-nonadienol; 3,7-dimethyl-7-methoxyoctan-2-ol; 9-decenol; 10-undecenol; 4-methyl-3-decen-5-ol;
the aliphatic aldehydes and acetals thereof such as e.g. hexanal; heptanal; octanal; nonanal; decanal; undecanal; dodecanal; tridecanal; 2-methyloctanal; 2-methylnonanal; (E)-2-hexenal; (Z)-4-heptenal; 2,6-dimethyl-5-heptenal; 10-undecenal; (E)-4-decenal; 2-dodecenal; 2,6,10-trimethyl-9-undecenal; 2,6,10 trimethyl-5,9-undecadienal; heptanal diethylacetal; 1,1-dimethoxy-2,2,5 trimethyl-4-hexene; citronellyloxyacetaldehyde; (E/Z)-1-(1-methoxypropoxy)-hex-3-ene; the aliphatic ketones and oximes thereof such as e.g. 2-heptanone; 2-octanone; 3-octanone; 2-nonanone; 5-methyl-3-heptanone; 5-methyl-3 heptanone oxime; 2,4,4,7-tetramethyl-6-octen-3-one; 6-methyl-5-hepten-2-one;
the aliphatic sulfur-containing compounds such as e.g. 3-methylthiohexanol; 3-methylthiohexyl acetate; 3-mercaptohexanol; 3-mercaptohexyl acetate; 3-mercaptohexyl butyrate; 3-acetylthiohexyl acetate; 1-menthene-8-thiol;
the aliphatic nitriles such as e.g. 2-nonenenitrile; 2-undecenenitrile; 2 tridecenenitrile; 3,12-tridecadienenitrile; 3,7-dimethyl-2,6-octadienenitrile; 3,7-dimethyl-6 octenenitrile; the esters of aliphatic carboxylic acids such as e.g. (E) and (Z)-3-hexenyl formate; ethyl acetoacetate; isoamyl acetate; hexyl acetate; 3,5,5-trimethylhexyl acetate; 3 methyl-2-butenyl acetate; (E)-2-hexenyl acetate; (E) and (Z)-3-hexenyl acetate; octyl acetate; 3-octyl acetate; 1-octen-3-yl acetate; ethyl butyrate; butyl butyrate; isoamyl butyrate; hexyl butyrate; (E) and (Z)-3-hexenyl isobutyrate; hexyl crotonate; ethyl isovalerate; ethyl 2-methylpentanoate; ethyl hexanoate; allyl hexanoate; ethyl heptanoate; allyl heptanoate; ethyl octanoate; ethyl (E,Z)-2,4-decadienoate; methyl 2-octinate; methyl 2-noninate; allyl 2-isoamyloxy acetate; methyl-3,7-dimethyl-2,6-octadienoate; 4-methyl-2-pentyl crotonate; the acyclic terpene alcohols such as e.g. geraniol; nerol; linalool; lavandulol; nerolidol; farnesol; tetrahydrolinalool; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2 ol; 3,7-dimethyl-4,6-octadien-3-ol; 3,7-dimethyl-1,5,7-octatrien-3-ol; 2,6-dimethyl-2,5,7-octatrien-1-ol; and the formates, acetates, propionates, isobutyrates, butyrates, isovalerates, pentanoates, hexanoates, crotonates, tiglinates and 3-methyl-2 butenoates thereof;
the acyclic terpene aldehydes and ketones such as e.g. geranial; neral; citronellal; 7 hydroxy-3,7-dimethyloctanal; 7 methoxy-3,7-dimethyloctanal; 2,6,10-trimethyl-9 undecenal; geranyl acetone; as well as the dimethyl and diethylacetals of geranial, neral, 7-hydroxy-3,7-dimethyloctanal; the cyclic terpene alcohols such as e.g. menthol; isopulegol; alpha-terpineol; terpine-4-ol; menthan-8-ol; menthan-1-ol; menthan-7-ol; borneol; isoborneol; linalool oxide; nopol; cedrol; ambrinol; vetiverol; guajol; and the formates, acetates, propionates, isobutyrates, butyrates, isovalerates, pentanoates, hexanoates, crotonates, tiglinates and 3-methyl-2-butenoates thereof;
the cyclic terpene aldehydes and ketones such as e.g. menthone; isomenthone; 8 mercaptomenthan-3-one; carvone; camphor; fenchone; alpha-ionone; beta-ionone; alpha-n-methylionone; beta-n-methylionone; alpha-isomethylionone; beta-isomethylionone; alpha-irone; alpha-damascone; beta-damascone; beta-damascenone; delta-damascone; gamma-damascone; 1-(2,4,4-trimethyl-2-cyclohexen-1-yl)-2-buten-1-one; 1,3,4,6,7,8a-hexahydro-1,1,5,5-tetramethyl-2H-2,4a-methano¬naphthalene-8(5H)-one; 2-methyl-4-(2,6,6-trimethyl-1-cyclohexen-1-yl)-2-butenal; nootkatone; dihydronootkatone; 4,6,8-megastigmatrien-3-one; alpha-sinensal; beta-sinensal; acetylated cedar wood oil (methyl cedryl ketone);
the cyclic alcohols such as e.g. 4-tert-butylcyclohexanol; 3,3,5-trimethylcyclohexanol; 3-isocamphylcyclohexanol; 2,6,9-trimethyl-Z2,Z5,E9-cyclododecatrien-1-ol; 2-isobutyl-4-methyltetrahydro-2H-pyran-4-ol;
the cycloaliphatic alcohols such as, e.g., alpha-3,3-trimethylcyclohexylmethanol; 1 (4-isopropylcyclohexyl)ethanol; 2-methyl-4-(2,2,3-trimethyl-3-cyclopent-1-yl)butanol; 2-methyl-4-(2,2,3 trimethyl-3-cyclopent-1-yl)-2-buten-1-ol; 2-ethyl-4-(2,2,3-trimethyl-3 cyclopent-1-yl)-2-buten-1-ol; 3-methyl-5-(2,2,3 trimethyl-3-cyclopent-1-yl)pentan-2 ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl)pentan-3-ol; 1-(2,2,6-trimethylcyclohexyl)hexan-3-ol;
the cyclic and cycloaliphatic ethers such as e.g. cineol; cedryl methyl ether; cyclododecyl methyl ether; 1,1-dimethoxycyclododecane; (ethoxy methoxy)cyclo-dodecane; alpha-cedrene epoxide; 3a,6,6,9a-tetramethyldodecahydronaphtho[2,1-b]furan; 3a-ethyl-6,6,9a-trimethyldodecahydro-naphtho[2,1-b]furan; 1,5,9-trimethyl-13-oxabicyclo-[10.1.0]trideca-4,8-diene; rose oxide; 2-(2,4-dimethyl-3-cyclohexen-1-yl)-5-methyl-5-(1-methylpropyl)-1,3-dioxane;
the cyclic and macrocyclic ketones such as e.g. 4-tert-butylcyclohexanone; 2,2,5 trimethyl-5-pentylcyclopentanone; 2-heptylcyclopentanone; 2-pentylcyclo-pentanone; 2-hydroxy-3-methyl-2-cyclopenten-1-one; 3-methyl-cis-2-penten-1-yl-2 cyclopenten-1-one; 3-methyl-2-pentyl-2-cyclopenten-1-one; 3-methyl-4-cyclopenta-decenone; 3-methyl-5-cyclopentadecenone; 3-methylcyclopentadecanone; 4-(1-ethoxyvinyl)-3,3,5,5-tetramethylcyclohexanone; 4-tert-pentylcyclohexanone; 5-cyclohexadecen-1-one; 6,7-dihydro-1,1,2,3,3-pentamethyl-4(5H)-indanone; 8-cyclo-hexadecen-1-one; 7-cyclohexadecen-1-one; (7/8)-cyclohexadecen-1-one; 9 cyclo-heptadecen-1-one; cyclopentadecanone; cyclohexadecanone;
the cycloaliphatic aldehydes such as e.g. 2,4-dimethyl-3-cyclohexenecarbaldehyde; 2 methyl-4-(2,2,6-trimethylcyclohexen-1-yl)-2-butenal; 4-(4-hydroxy-4-methylpentyl)-3 cyclohexene carbaldehyde; 4-(4-methyl-3-penten-1-yl)-3-cyclohexenecarbaldehyde; the cycloaliphatic ketones such as e.g. 1-(3,3-dimethylcyclohexyl)-4-penten-1-one; 2,2 dimethyl-1-(2,4-dimethyl-3-cyclohexen-1-yl)-1-propanone; 1-(5,5-dimethyl-1 cyclo-hexen-1-yl)-4-penten-1-one; 2,3,8,8-tetramethyl-1,2,3,4,5,6,7,8-octahydro-2-naphthalenyl methyl ketone; methyl 2,6,10-trimethyl-2,5,9-cyclododecatrienyl ketone; tert-butyl (2,4-dimethyl-3-cyclohexen-1-yl) ketone;
the esters of cyclic alcohols such as e.g. 2-tert-butylcyclohexyl acetate; 4-tert-butylcyclohexyl acetate; 2-tert-pentylcyclohexyl acetate; 4-tert-pentylcyclohexyl acetate; 3,3,5-trimethylcyclohexyl acetate; decahydro-2-naphthyl acetate; 2-cyclopentylcyclopentyl crotonate; 3-pentyltetrahydro-2H-pyran-4-yl acetate; decahydro-2,5,5,8a-tetramethyl-2-naphthyl acetate; 4,7-methano-3a,4,5,6,7,7a-hexahydro-5 or 6-indenyl acetate; 4,7-methano-3a,4,5,6,7,7a-hexahydro-5 or 6 indenyl propionate; 4,7-methano-3a,4,5,6,7,7a-hexahydro-5 or 6-indenyl isobutyrate; 4,7 methanooctahydro-5 or 6-indenyl acetate; the esters of cycloaliphatic alcohols such as e.g. 1-cyclohexylethyl crotonate;
the esters of cycloaliphatic carboxylic acids such as e.g. allyl 3-cyclohexylpropionate; allyl cyclohexyloxyacetate; cis and trans-methyl dihydrojasmonate; cis and trans-methyl jasmonate; methyl 2-hexyl-3-oxocyclopentanecarboxylate; ethyl 2-ethyl-6,6 dimethyl-2-cyclohexenecarboxylate; ethyl 2,3,6,6-tetramethyl-2 cyclohexene-carboxylate; ethyl 2-methyl-1,3-dioxolane-2-acetate;
the araliphatic alcohols such as, e.g., benzyl alcohol; 1-phenylethyl alcohol, 2 phenylethyl alcohol, 3-phenylpropanol; 2-phenylpropanol; 2-phenoxyethanol; 2,2-dimethyl-3-phenylpropanol; 2,2-dimethyl-3-(3-methylphenyl)propanol; 1,1-dimethyl-2 phenylethyl alcohol; 1,1-dimethyl-3-phenylpropanol; 1-ethyl-1-methyl-3-phenylpropanol; 2-methyl-5-phenylpentanol; 3-methyl-5-phenylpentanol; 3-phenyl-2-propen-1-ol; 4-methoxy¬benzyl alcohol; 1-(4-isopropylphenyl)ethanol;
the esters of araliphatic alcohols and aliphatic carboxylic acids such as, e.g., benzyl acetate; benzyl propionate; benzyl isobutyrate; benzyl isovalerate; 2-phenylethyl acetate; 2-phenylethyl propionate; 2-phenylethyl isobutyrate; 2 phenylethyl isovalerate; 1 phenylethyl acetate; alpha-trichloromethylbenzyl acetate; alpha,alpha-dimethylphenylethyl acetate; alpha,alpha-dimethylphenylethyl butyrate; cinnamyl acetate; 2-phenoxyethyl isobutyrate; 4-methoxybenzyl acetate;
the araliphatic ethers such as e.g. 2-phenylethyl methyl ether; 2 phenylethyl isoamyl ether; 2-phenylethyl 1-ethoxyethyl ether; phenylacetaldehyde dimethyl acetal; phenylacetaldehyde diethyl acetal; hydratropaaldehyde dimethyl acetal; phenylacetaldehyde glycerol acetal; 2,4,6-trimethyl-4-phenyl-1,3-dioxane; 4,4a,5,9b-tetrahydroindeno[1,2-d]-m-dioxine; 4,4a,5,9b-tetrahydro-2,4-dimethylindeno[1,2-d]-m dioxine;
the aromatic and araliphatic aldehydes such as e.g. benzaldehyde; phenylacetaldehyde; 3-phenylpropanal; hydratropaaldehyde; 4-methylbenzaldehyde; 4 methyl phenylacetaldehyde; 3-(4-ethylphenyl)-2,2-dimethylpropanal; 2-methyl-3-(4-isopropylphenyl)propanal; 2-methyl-3-(4-tert-butylphenyl)propanal; 2-methyl-3-(4-isobutylphenyl)propanal; 3-(4-tert-butylphenyl)propanal; cinnamaldehyde; alpha-butylcinnamaldehyde; alpha-amylcinnamaldehyde; alpha-hexylcinnamaldehyde; 3 methyl-5-phenylpentanal; 4-methoxybenzaldehyde; 4-hydroxy-3 methoxy-benzaldehyde; 4-hydroxy-3-ethoxybenzaldehyde; 3,4-methylenedioxybenzaldehyde; 3,4-dimethoxybenzaldehyde; 2-methyl-3-(4-methoxyphenyl)propanal; 2-methyl-3-(4-methylenedioxyphenyl)propanal;
the aromatic and araliphatic ketones such as e.g. acetophenone; 4-methylacetophenone; 4-methoxyacetophenone; 4-tert-butyl-2,6-dimethylaceto-phenone; 4-phenyl-2-butanone; 4-(4-hydroxyphenyl)-2-butanone; 1-(2-naphthalenyl)-ethanone; 2-benzofuranylethanone; (3-methyl-2-benzofuranyl)ethanone; benzophenone; 1,1,2,3,3,6-hexamethyl-5-indanyl methyl ketone; 6-tert-butyl-1,1 dimethyl-4 indanyl methyl ketone; 1-[2,3-dihydro-1,1,2,6-tetramethyl-3-(1-methylethyl)-1H-5 indenyl]ethanone; 5,6,7,8-tetrahydro-3,5,5,6,8,8-hexamethyl-2-acetonaphthone;
the aromatic and araliphatic carboxylic acids and esters thereof such as e.g. benzoic acid; phenylacetic acid; methyl benzoate; ethyl benzoate; hexyl benzoate; benzyl benzoate; methyl phenylacetate; ethyl phenylacetate; geranyl phenylacetate; phenylethyl phenylacetate; methyl cinnamate; ethyl cinnamate; benzyl cinnamate; phenylethyl cinnamate; cinnamyl cinnamate; allyl phenoxyacetate; methyl salicylate; isoamyl salicylate; hexyl salicylate; cyclohexyl salicylate; cis-3-hexenyl salicylate; benzyl salicylate; phenylethyl salicylate; methyl 2,4-dihydroxy-3,6-dimethylbenzoate; ethyl 3-phenylglycidate; ethyl 3-methyl-3-phenylglycidate;
the nitrogen-containing aromatic compounds such as e.g. 2,4,6-trinitro-1,3-dimethyl-5 tert-butylbenzene; 3,5-dinitro-2,6-dimethyl-4-tert-butylacetophenone; cinnamonitrile; 3 methyl-5-phenyl-2-pentenonitrile; 3-methyl-5-phenylpentanonitrile; methyl anthranilate; methyl-N-methylanthranilate; Schiff bases of methyl anthranilate with 7 hydroxy-3,7-dimethyloctanal, 2-methyl-3-(4-tert-butylphenyl)propanal or 2,4 dimethyl-3-cyclohexenecarbaldehyde; 6-isopropylquinoline; 6-isobutylquinoline; 6-sec-butylquinoline; 2-(3-phenylpropyl)pyridine; indole; skatole; 2-methoxy-3 isopropyl-pyrazine; 2-isobutyl-3-methoxypyrazine; the phenols, phenyl ethers and phenyl esters such as e.g. estragole; anethole; eugenol; eugenyl methyl ether; isoeugenol; isoeugenyl methyl ether; thymol; carvacrol; diphenyl ether; beta-naphthyl methyl ether; beta-naphthyl ethyl ether; beta-naphthyl isobutyl ether; 1,4-dimethoxybenzene; eugenyl acetate; 2-methoxy-4-methylphenol; 2 ethoxy-5-(1-propenyl)phenol; p-cresyl phenylacetate;
the heterocyclic compounds such as e.g. 2,5-dimethyl-4-hydroxy-2H-furan-3-one; 2 ethyl-4-hydroxy-5-methyl-2H-furan-3-one; 3-hydroxy-2-methyl-4H-pyran-4-one; 2 ethyl-3-hydroxy-4H-pyran-4-one;
the lactones such as e.g. 1,4-octanolide; 3-methyl-1,4-octanolide; 1,4-nonanolide; 1,4-decanolide; 8-decen-1,4-olide; 1,4-undecanolide; 1,4-dodecanolide; 1,5-decanolide; 1,5-dodecanolide; 4-methyl-1,4-decanolide; 1,15-pentadecanolide; cis and trans-11-pentadecen-1,15-olide; cis and trans-12-pentadecen-1,15-olide; 1,16-hexadecanolide; 9-hexadecen-1,16-olide; 10-oxa-1,16-hexadecanolide; 11-oxa-1,16-hexadecanolide; 12-oxa-1,16-hexadecanolide; ethylene 1,12-dodecanedioate; ethylene 1,13-tridecanedioate; coumarin; 2,3-dihydrocoumarin; octahydrocoumarin.

In a preferred embodiment, the at least one non-aroma chemical carrier (ii) is selected from the group consisting of surfactants, oil components, antioxidants, deodorant-active agents and solvents.

In the context of the presently claimed invention, a “solvent” serves for the dilution of the compound of formula (1) and/or (4) to be used according to the invention and/or any further component of the composition without having its own aroma.

The amount of solvent(s) is selected depending on the composition.

In yet another preferred embodiment, the solvent is selected from the group consisting of ethanol, isopropanol, diethylene glycol monoethyl ether, glycerol, propylene glycol, 1,2 butylene glycol, dipropylene glycol, triethyl citrate and isopropyl myristate.

In yet another preferred embodiment, the solvent is present in the composition in an amount of 0.01 wt. % to 99.0 wt. %, more preferably in an amount of 0.05 wt. % to 95.0 wt. %, yet more preferably in an amount of 0.1 wt. % to 80.0 wt. %, most preferably 0.1 wt. % to 70.0 wt. %, particularly in an amount of 0.1 wt. % to 60.0 wt. %, based on the total weight of the composition.

In yet another preferred embodiment of the invention, the composition comprises 0.05 wt. % to 10 wt. %, more preferably 0.1 wt. % to 5 wt. %, yet more preferably 0.2 wt. % to 3 wt. % solvent(s), based on the total weight of the composition. In yet another preferred embodiment of the invention, the composition comprises 20 wt. % to 70 wt. %, more preferably 25 wt. % to 50 wt. % of solvent(s), based on the total weight of the composition.

One embodiment of the invention is directed to a composition comprising the compound of formula (1) and/or (4) and at least one oil component.

In a preferred embodiment, the oil components are present in an amount of 0.1 to 80 wt. %, more preferably 0.5 to 70 wt. %, yet more preferably 1 to 60 wt. %, even more preferably 1 to 50 wt. %, particularly 1 to 40 wt. %, more particularly 5 to 25 wt. % and specifically 5 to 15 wt. %, based on the total weight of the composition.

The oil components may be selected, for example, from Guerbet alcohols based on fatty alcohols containing 6 to 18, preferably 8 to 10, carbon atoms and other additional esters, such as myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of C18-C38 alkyl-hydroxycarboxylic acids with linear or branched C6-C22 fatty alcohols, more especially dioctyl malate, esters of linear and/or branched fatty acids with polyhydric alcohols (for example propylene glycol, dimer dial or trimer triol), triglycerides based on C6-C10 fatty acids, liquid mono-, di- and triglyceride mixtures based on C6-C18 fatty acids, esters of C6-C22 fatty alcohols and/or Guerbet alcohols with aromatic carboxylic acids, more particularly benzoic acid, esters of dicarboxylic acids with polyols containing 2 to 10 carbon atoms and 2 to 6 hydroxyl groups, vegetable oils, branched primary alcohols, substituted cyclohexanes, linear and branched C6-C22 fatty alcohol carbonates such as, for example, dicaprylyl carbonate (Cetiol® CC), Guerbet carbonates based on fatty alcohols containing 6 to 18, preferably 8 to 10, carbon atoms, esters of benzoic acid with linear and/or branched C6 to C22 alcohols (for example Finsolv® TN), linear or branched, symmetrical or nonsymmetrical dialkyl ethers containing 6 to 22 carbon atoms per alkyl group such as, for example, dicaprylyl ether (Cetiol® OE), ring opening products of epoxidized fatty acid esters with polyols and hydrocarbons or mixtures thereof.

It is to be understood that antioxidants are able to inhibit or prevent the undesired changes in the compositions to be protected caused by oxygen effects and other oxidative processes. The effect of the antioxidants consists in most cases in them acting as free-radical scavengers for the free radicals which arise during autoxidation.

In a preferred embodiment, the antioxidant is selected from the group consisting of
amino acids (for example glycine, alanine, arginine, serine, threonine, histidine, tyrosine, tryptophan) and derivatives thereof,
imidazoles (e.g. urocanic acid) and derivatives thereof,
peptides, such as D,L-carnosine, D-carnosine, L-carnosine (=β-Alanyl-L-histidin) and derivatives thereof
carotenoids, carotenes (e.g. alpha-carotene, beta-carotene, lycopene, lutein) or derivatives thereof,
chlorogenic acid and derivatives thereof,
lipoic acid and derivatives thereof (for example dihydrolipoic acid),
auro-thioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, gamma-linoleyl, cholesteryl and glyceryl esters thereof) and salts thereof,
dilauryl thiodipropionate, distearyl thiodipropionate, thiodipropionic acid and derivatives thereof (esters, ethers, peptides, lipids, nucleotides, nucleosides and salts),
sulfoximine compounds (for example buthionine sulfoximines, homocysteine sulfoximine, buthionine sulfones, penta-, hexa-, heptathionine sulfoximine)
(metal) chelating agents (e.g. alpha-hydroxy fatty acids, palmitic acid, phytic acid, lactoferrin),
alpha-hydroxy acids (for example citric acid, lactic acid, malic acid),
humic acid, bile acid, bile extracts, bilirubin, biliverdin, boldin (=alkaloid from the plant Peumus boldus, boldo extract,
EDTA, EGTA and derivatives thereof,
unsaturated fatty acids and derivatives thereof (e.g. gamma-linolenic acid, linoleic acid, oleic acid),
folic acid and derivatives thereof,
ubiquinone and ubiquinol and derivatives thereof,
vitamin C and derivatives (for example ascorbyl palmitate, Mg ascorbyl phosphate, ascorbyl acetate),
tocopherols and derivatives (for example vitamin E acetate),
vitamin A and derivatives (for example vitamin A palmitate), coniferyl benzoate of gum benzoin, rutic acid and derivatives thereof, alpha-glycosylrutin, ferulic acid, furfurylideneglucitol,
butylhydroxytoluene (BHT), butylhydroxyanisole (BHA)
nordihydroguaiacic acid, nordihydroguaiaretic acid, trihydroxybutyrophenone, uric acid and derivatives thereof, mannose and derivatives thereof,
superoxide dismutase
zinc and derivatives thereof (for example ZnO, ZnSO4),
selenium and derivatives thereof (for example selenomethionine) and
stilbenes and derivatives thereof (e.g. stilbene oxide, trans-stilbene oxide)

In a preferred embodiment, the antioxidant is selected from the group consisting of pentaerythrityl, tetra-di-t-butyl-hydroxyhydrocinnamate, nordihydroguaiaretic acid, ferulic acid, resveratrol, propyl gallate, butylhydroxytoluene (BHT), butylhydroxyanisole (BHA), ascorbyl palmitate and tocopherol.

In yet another preferred embodiment, the compositions according to the presently claimed invention can comprise the anti-oxidant in an amount of 0.001 to 25 wt.-%, preferably 0.005 to 10 wt.-%, more preferably 0.01 to 8 wt.-%, yet more preferably 0.025 to 7 wt.-%, even more preferably 0.05 to 5 wt.-%, based on the total weight of the composition.

Deodorizing compositions (deodorants and antiperspirants) counteract, mask or eliminate body odors. Body odors are formed through the action of skin bacteria on apocrine perspiration which results in the formation of unpleasant-smelling degradation products.

One embodiment of the invention is therefore directed to a composition comprising the compound of formula (1) and/or (4) and at least one deodorant-active agent. In a preferred embodiment, the deodorant-active agent is selected from the groups consisting of anti-perspirants, esterase inhibitors and antibacterial agents.

Suitable antiperspirant is selected from the group consisting of salts of aluminum, zirconium or zinc. Examples are aluminum chloride, aluminum chlorohydrate, aluminum dichlorohydrate, aluminum sesquichlorohydrate and complex compounds thereof, for example with 1,2-propylene glycol, aluminum hydroxyallantoinate, aluminum chloride tartrate, aluminum zirconium trichlorohydrate, aluminum zirconium tetrachlorohydrate, aluminum zirconium pentachlorohydrate and complex compounds thereof, for example with amino acids, such as glycine. Aluminum chlorohydrate, aluminum zirconium tetrachlorohydrate, aluminum zirconium pentachlorohydrate and complex compounds thereof are preferably used.

In a preferred embodiment, the anti-perspirant is selected from the group consisting of aluminum chloride, aluminum chlorohydrate, aluminum dichlorohydrate, aluminum sesquichlorohydrate, aluminum hydroxyallantoinate, aluminum chloride tartrate, aluminum zirconium trichlorohydrate, aluminum zirconium tetrachlorohydrate and aluminum zirconium pentachlorohydrate.

Where perspiration is present in the underarm region, extracellular enzymes-esterases, mainly proteases and/or lipases are formed by bacteria and split the esters present in the perspiration, releasing odors in the process. Suitable esterase inhibitors are for example trialkyl citrates, such as trimethyl citrate, tripropyl citrate, triisopropyl citrate, tributyl citrate and, in particular, triethyl citrate. Esterase inhibitors inhibit enzyme activity and thus reduce odor formation. The free acid is probably released by the cleavage of the citric acid ester and reduces the pH value of the skin to such an extent that the enzymes are inactivated by acylation. Other esterase inhibitors are sterol sulfates or phosphates such as, for example, lanosterol, cholesterol, campesterol, stigmasterol and sitosterol sulfate or phosphate, dicarboxylic acids and esters thereof, for example glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and esters thereof, for example citric acid, malic acid, tartaric acid or tartaric acid diethyl ester, and zinc glycinate.

In a preferred embodiment, the esterase inhibitor is selected from the group consisting of trimethyl citrate, tripropyl citrate, triisopropyl citrate, tributyl citrate triethyl citrate, lanosterol, cholesterol, campesterol, stigmasterol, sitosterol sulfate, sitosterol phosphate, glutaric acid, glutaric acid monoethyl ester, glutaric acid diethyl ester, adipic acid, adipic acid monoethyl ester, adipic acid diethyl ester, malonic acid, malonic acid diethyl ester, citric acid, malic acid, tartaric acid, tartaric acid diethyl ester and zinc glycinate.

The compositions according to the presently claimed invention comprises the esterase inhibitor in the range of 0.01 to 20 wt.-%, preferably 0.1 to 10 wt.-% and more particularly 0.5 to 5 wt.-%, based on the total weight of the composition.

The term “anti-bacterial agents” as used herein encompasses substances which have bactericidal and/or bacteriostatic properties. Typically these substances act against gram-positive bacteria such as, for example, 4-hydroxybenzoic acid and salts and esters thereof, N-(4-chlorophenyl)-N′-(3,4-dichlorophenyl)-urea, 2,4,4′-trichloro-2′-hydroxydiphenylether (triclosan), 4-chloro-3,5-dimethylphenol, 2,2′-methylene-bis-(6-bromo-4-chlorophenol), 3-methyl-4-(1-methylethyl)-phenol, 2-benzyl-4-chlorophenol, 3-(4-chlorophenoxy)-propane-1,2-diol, 3-iodo-2-propinyl butyl carbamate, chlorhexidine, 3,4,4′-trichlorocarbanilide (TTC), phenoxyethanol, glycerol monocaprate, glycerol monocaprylate, glycerol monolaurate (GML), diglycerol monocaprate (DMC), salicylic acid-N-alkylamides such as, for example, salicylic acid-n-octyl amide or salicylic acid-n-decyl amide.

In a preferred embodiment, the antibacterial agent is selected from the group consisting of chitosan, phenoxyethanol, 5-chloro-2-(2,4-dichlorophenoxy)-phenol, 4-hydroxybenzoic acid and salts and esters thereof, N-(4-chlorophenyl)-N′-(3,4-dichlorophenyl)-urea, 2,4,4′-trichloro-2′-hydroxydiphenylether (triclosan), 4-chloro-3,5-dimethylphenol, 2,2′-methylene-bis-(6-bromo-4-chlorophenol), 3-methyl-4-(1-methylethyl)-phenol, 2-benzyl-4-chlorophenol, 3-(4-chlorophenoxy)-propane-1,2-diol, 3-iodo-2-propinyl butyl carbamate, chlorhexidine, 3,4,4′-trichlorocarbanilide (TTC), phenoxyethanol, glycerol monocaprate, glycerol monocaprylate, glycerol monolaurate (GML), diglycerol monocaprate (DMC), salicylic acid-N-alkylamides.

The composition according to the presently claimed invention comprises the antibacterial agent in the range of 0.01 to 5 wt. % and preferably 0.1 to 2 wt.-%, based on the total weight of the composition.

In a preferred embodiment, the composition preferably comprises a surfactant. Due to the characteristic fragrance property of the compound of formula (1) and/or (4) and its substantivity, tenacity as well as stability, it can especially be used to provide an odor, preferably a fragrance impression or aroma impression to surfactant-containing compositions such as, for example, cleaners (in particular laundry care products and all-purpose cleaners). It can preferably be used to impart a long-lasting a flowery and/or a green and/or a sweet note and/or a woody note and/or a rooty note and/or a violet note odiferous impression to a surfactant comprising composition.

In a preferred embodiment, the surfactant is selected from the group consisting of anionic, non-ionic, cationic, amphoteric and zwitterionic surfactants. In yet another preferred embodiment, the surfactant is an anionic surfactant.

The compositions according to the presently claimed invention can thus preferably comprise at least one surfactant. The surfactant(s) may be selected from anionic, non-ionic, cationic and/or amphoteric or zwitterionic surfactants. Surfactant-containing compositions, such as for example shower gels, foam baths, shampoos, etc., preferably contain at least one anionic surfactant.

The compositions according to the invention usually contain the surfactant(s), in the aggregate, in an amount of 0 to 40 wt. %, preferably 0 to 20 wt. %, more preferably 0.1 to 15 wt. %, and particularly 0.1 to 10 wt. %, based on the total weight of the composition. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partly oxidized alk(en)yl oligoglycosides or glucuronic acid derivatives, fatty acid-N-alkyl glucamides, protein hydrolysates (particularly wheat-based vegetable products), polyol fatty acid esters, sugar esters, sorbitan esters, polysorbates and amine oxides. If the nonionic surfactants contain polyglycol ether chains, they may have a conventional homolog distribution, although they preferably have a narrow-range homolog distribution.

Zwitterionic surfactants are surface-active compounds which contain at least one quaternary ammonium group and at least one COO (−) or SO3(−) group in the molecule. Particularly suitable zwitterionic surfactants are the so-called betaines, such as the N-alkyl-N,N-dimethyl ammonium glycinates, for example, cocoalkyl dimethyl ammonium glycinate, N-acylaminopropyl-N,N-dimethyl ammonium glycinates, for example, cocoacylaminopropyl dimethyl ammonium glycinate, and 2-alkyl-3-carboxymethyl-3-hydroxyethyl imidazolines, containing 8 to 18 carbon atoms in the alkyl or acyl group, and cocoacylaminoethyl hydroxyethyl carboxymethyl glycinate. The fatty acid amide derivative known under the CTFA name of Cocamidopropyl Betaine is particularly preferred.

Ampholytic surfactants are also suitable, particularly as co-surfactants. Ampholytic surfactants are surface-active compounds which, in addition to a C8 to C18 alkyl or acyl group, contain at least one free amino group and at least one —COOH or —SO3H group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-cocoalkylaminopropionate, cocoacylaminoethyl aminopropionate and acyl sarcosine.

Anionic surfactants are characterized by a water-solubilizing anionic group such as, for example, a carboxylate, sulfate, sulfonate or phosphate group and a lipophilic group.

Dermatologically safe anionic surfactants are known to the practitioner in large numbers from relevant textbooks and are commercially available. They are, in particular, alkyl sulfates in the form of their alkali metal, ammonium or alkanolammonium salts, alkylether sulfates, alkylether carboxylates, acyl isethionates, acyl sarcosinates, acyl taurines containing linear C12-C18 alkyl or acyl groups and sulfosuccinates and acyl glutamates in the form of their alkali metal or ammonium salts.

Particularly suitable cationic surfactants are quaternary ammonium compounds, preferably ammonium halides, more especially chlorides and bromides, such as alkyl trimethyl ammonium chlorides, dialkyl dimethyl ammonium chlorides and trialkyl methyl ammonium chlorides, for example, cetyl trimethyl ammonium chloride, stearyl trim ethyl ammonium chloride, distearyl dimethyl ammonium chloride, lauryl dimethyl ammonium chloride, lauryl dimethyl benzyl ammonium chloride and tricetyl methyl ammonium chloride. In addition, the readily biodegradable quaternary ester compounds, such as, for example, the dialkyl ammonium methosulfates and methyl hydroxyalkyl dialkoyloxyalkyl ammonium methosulfates marketed under the name of Stepantexe and the corresponding products of the Dehyquart® series, may be used as cationic surfactants. “Esterquats” are generally understood to be quaternized fatty acid triethanolamine ester salts. They can provide the compositions with particular softness. They are known substances which are prepared by the relevant methods of organic chemistry. Other cationic surfactants suitable for use in accordance with the invention are the quaternized protein hydrolysates.

One embodiment of the presently claimed invention is directed to a composition which is selected from the group consisting of perfume compositions, body care compositions, hygiene articles, cleaning compositions, textile detergent compositions, compositions for scent dispensers, foods, food supplements, pharmaceutical compositions and crop protection compositions.

Said composition is preferably an aroma chemical composition, more preferably a fragrance composition.

Suitable compositions are for example perfume compositions, body care compositions (including cosmetic compositions and products for oral and dental hygiene), hygiene articles, cleaning compositions (including dishwashing compositions), textile detergent compositions, compositions for scent dispensers, foods, food supplements, pharmaceutical compositions and crop protection compositions.

Perfume compositions can be selected from fine fragrances, air fresheners in liquid form, gel-like form or a form applied to a solid carrier, aerosol sprays, scented cleaners, perfume candles and oils, such as lamp oils or oils for massage.

Examples for fine fragrances are perfume extracts, Eau de Parfums, Eau de Toilettes, Eau de Colognes, Eau de Solide and Extrait Parfum.

Body care compositions include cosmetic compositions and products for oral and dental hygiene, and can be selected from after-shaves, pre-shave products, splash colognes, solid and liquid soaps, shower gels, shampoos, shaving soaps, shaving foams, bath oils, cosmetic emulsions of the oil-in-water type, of the water-in-oil type and of the water-in-oil-in-water type, such as e.g. skin creams and lotions, face creams and lotions, sunscreen creams and lotions, after-sun creams and lotions, hand creams and lotions, foot creams and lotions, hair removal creams and lotions, after-shave creams and lotions, tanning creams and lotions, hair care products such as e.g. hairsprays, hair gels, setting hair lotions, hair conditioners, hair shampoo, permanent and semi-permanent hair colorants, hair shaping compositions such as cold waves and hair smoothing compositions, hair tonics, hair creams and hair lotions, deodorants and antiperspirants such as e.g. underarm sprays, roll-ons, deodorant sticks and deodorant creams, products of decorative cosmetics such as e.g. eye-liners, eye-shadows, nail varnishes, make-ups, lipsticks and mascara, and products for oral and dental hygiene, such as toothpaste, dental floss, mouth wash, breath fresheners, dental foam, dental gels and dental strips.

Hygiene articles can be selected from joss sticks, insecticides, repellents, propellants, rust removers, perfumed freshening wipes, armpit pads, baby diapers, sanitary towels, toilet paper, cosmetic wipes, pocket tissues, dishwasher and deodorizer.

Cleaning compositions, such as, e.g., cleaners for solid surfaces, can be selected from perfumed acidic, alkaline and neutral cleaners, such as, e.g., floor cleaners, window cleaners, dishwashing compositions both for handwashing and machine washing use, bath and sanitary cleaners, scouring milk, solid and liquid toilet cleaners, powder and foam carpet cleaners, waxes and polishes such as furniture polishes, floor waxes, shoe creams, disinfectants, surface disinfectants and sanitary cleaners, brake cleaners, pipe cleaners, limescale removers, grill and oven cleaners, algae and moss removers, mold removers, facade cleaners.

Textile detergent compositions can be selected from liquid detergents, powder detergents, laundry pretreatments such as bleaches, soaking agents and stain removers, fabric softeners, washing soaps, washing tablets.

Food means a raw, cooked, or processed edible substance, ice, beverage or ingredient used or intended for use in whole or in part for human consumption, or chewing gum, gummies, jellies, and confectionaries.

A food supplement is a product intended for ingestion that contains a dietary ingredient intended to add further nutritional value to the diet. A dietary ingredient may be one, or any combination, of the following substances: a vitamin, a mineral, an herb or other botanical, an amino acid, a dietary substance for use by people to supplement the diet by increasing the total dietary intake, a concentrate, metabolite, constituent, or extract. Food supplements may be found in many forms such as tablets, capsules, soft gels, gel caps, liquids, or powders.

Pharmaceutical compositions comprise compositions which are intended for use in the diagnosis, cure, mitigation, treatment, or prevention of disease as well as articles (other than food) intended to affect the structure or any function of the body of man or other animals.

Crop protection compositions comprise compositions which are intended for the managing of plant diseases, weeds and other pests (both vertebrate and invertebrate) that damage agricultural crops and forestry.

In a preferred embodiment, the composition further comprises at least one auxiliary agent selected from the group consisting of preservatives, abrasives, anti-acne agents, agents to combat skin aging, anti-cellulite agents, antidandruff agents, anti-inflammatory agents, irritation-preventing agents, irritation-alleviating agents, astringents, sweat-inhibiting agents, antiseptics, anti-statics, binders, buffers, carrier materials, chelating agents, cell stimulants, care agents, hair removal agents, emulsifiers, enzymes, essential oils, fibers, film formers, fixatives, foam formers, foam stabilizers, substances for preventing foaming, foam boosters, fungicides, gelling agents, gel-forming agents, hair care agents, hair shaping agents, hair smoothing agents, moisture-donating agents, moisturizing substances, humectant substances, bleaching agents, strengthening agents, stain removal agents, optical brighteners, impregnating agents, soil repellents, friction-reducing agents, lubricants, moisturizing creams, ointments, opacifiers, plasticizers, covering agents, polish, shine agents, polymers, powders, proteins, refatting agents, exfoliating agents, silicones, skin-calming agents, skin-cleansing agents, skin care agents, skin-healing agents, skin lightening agents, skin-protective agents, skin-softening agents, cooling agents, skin-cooling agents, warming agents, skin-warming agents, stabilizers, UV-absorbent agents, UV filters, fabric softeners, suspending agents, skin-tanning agents, thickeners, vitamins, waxes, fats, phospholipids, saturated fatty acids, mono or polyunsaturated fatty acids, hydroxy acids, polyhydroxy fatty acids, liquefiers, dyes, color-protection agents, pigments, anti-corrosives, polyols, electrolytes and silicone derivatives.

For example, the method can be carried out by mixing the alpha-ionylideneethane without or with alpha-ionone and:
(i) at least one additional aroma chemical different from alpha-ionylideneethane or alpha-ionone, or
(ii) at least one non-aroma chemical carrier, or
(iii) both of (i) and (ii).

The invention is also directed to a method for modifying the aroma character (e.g., scent character) of an aroma chemical composition such as, e.g., a fragranced composition, in particular a fragranced ready-to-use composition, wherein the method comprises incorporating the alpha-ionylideneethane without or with alpha-ionone into an aroma chemical composition such as, e.g., into a fragranced composition, in particular into a fragranced ready-to-use composition.

In particular, the invention is directed to a method of preparing a perfume composition, body care composition, hygiene article, cleaning composition, textile detergent composition, composition for scent dispensers, food, food supplement, pharmaceutical composition or crop protection composition, comprising including the alpha-ionylideneethane without or with alpha-ionone in a perfume composition, body care composition, hygiene article, cleaning composition, textile detergent composition, composition for scent dispensers, food, food supplement, pharmaceutical composition or crop protection composition.

In one embodiment the invention is directed to a method for imparting a note reminiscent of sweet, floral, violet, orris, rooty and/or woody to a perfume composition, body care composition, hygiene article, cleaning composition, textile detergent composition, composition for scent dispensers, food, food supplement, pharmaceutical composition or crop protection composition, which comprises including an alpha-ionylideneethane without or with alpha-ionone in a perfume composition, body care composition, hygiene article, cleaning composition, textile detergent composition, composition for scent dispensers, food, food supplement, pharmaceutical composition or crop protection composition.

It is preferred that the methods of the invention are or comprise fermentative methods.

In addition, the present invention relates to an aroma compound and/or fragrance composition and/or perfumed or fragranced product, comprising:
i) at least an alpha-ionylideneethane as defined herein, preferably an alpha-ionylideneethane as defined in claim 1 or 2;
ii) optionally, at least one further aroma compound different from i), and
iii) optionally, at least one diluent.

Preferably, the aroma compound and/or fragrance composition and/or perfumed or fragranced product of the present invention comprises i) and ii), or i) and iii), more preferably i), ii) and iii).

The present invention also pertains to a perfumed or fragranced product comprising at least an alpha-ionylideneethane as defined herein, preferably an alpha-ionylideneethane having a note of Floral-Violet and/or Woody-Orris/Iris Root and more preferably 2Z,4E-alpha-ionylideneethane.

For instance, the alpha-ionylideneethane as defined herein, preferably an alpha-ionylideneethane as defined herein, can be used in compositions selected from perfumes, detergents and cleaning compositions, cosmetic agents, body care agents, hygiene articles, products for oral and dental hygiene, scent dispensers, and other compositions and products defined herein.

As used herein, the singular forms “a”, “an” and “the” include both singular and plural reference unless the context clearly dictates otherwise. By way of example, “a cell” refers to one or more than one cell.

As used herein, the term “about” when qualifying a value of a stated item, number, percentage, or term refers to a range of plus or minus 10 percent, 9 percent, 8 percent, 7 percent, 6 percent, 5 percent, 4 percent, 3 percent, 2 percent or 1 percent of the value of the stated item, number, percentage, or term. Preferred is a range of plus or minus 10 percent.

The terms “comprising”, “comprises” and “comprised of” as used herein are synonyms with “including”, “includes” or “containing”, “contains”, and are inclusive or open-ended and do not exclude additional, non-recited members, elements or method steps. Evidently, the term “comprising” encompasses the term “consisting of”. More specifically, the term “comprise” as used herein means that the claim encompasses all the listed elements or method steps, but may also include additional, unnamed elements or method steps. For example, a method comprising steps a), b) and c) encompasses, in its narrowest sense, a method which consists of steps a), b) and c). The phrase “consisting of” means that the composition (or kit, or method) has the recited elements (or steps) and no more. In contrast, the term “comprises” can encompass also a method including further steps, e.g., steps d) and e), in addition to steps a), b) and c).

In case numerical ranges are used herein such as “in a concentration between 1 and 5 micromolar”, the range includes not only 1 and 5 micromolar, but also any numerical value in between 1 and 5 micromolar, for example, 2, 3 and 4 micromolar. Per definition, the term “in vitro” means outside the living body and in an artificial environment. Accordingly, the term “in vitro” as used herein denotes outside, or external to, the animal or human body. The term “in vitro” as used herein should be understood to include “ex vivo”. The term “ex vivo” typically refers to tissues or cells removed from an animal or human body and maintained or propagated outside the body, e.g., in a culture vessel. The term “in vivo” as used herein denotes inside, or internal to, the animal or human body.

Per definition, the term “terpenes” comprises the hydrocarbons only, being composed of carbon and hydrogen. In contrast, the term “terpenoids” refers to terpenes containing additional functional groups, resulting in derivatives such as alcohols, aldehydes, ketones, and acids; see, e.g., Flavours and Fragrances: Chemistry, Bioprocessing and Sustainability R G Berger; Black et al., Terpenoids and their role in wine flavour: recent advances. Australian Journal of Grape and Wine Research 21, 582-600, 2015; Zhou & Pichersky, More is better: the diversity of terpene metabolism in plants. Current Opinion in Plant Biology 2020, 55:1-10; Degenhardt J, Köllner TG, Gershenzon J (2009) Monoterpene and sesquiterpene synthases and the origin of terpene skeletal diversity in plants. Phytochemistry 70(15):1621-1637). In the scientific literature, the term “terpene” is frequently used interchangeably with the term “terpenoid”, although they have different meanings. As used herein, the term “terpenes”, comprises both hydrocarbons and their functionalized derivatives.

Sesquiterpenes are C15-terpenoids built from three isoprene units. Like monoterpenes, sesquiterpenes may be acyclic or contain rings, including many unique combinations. They are found particularly in higher plants and in many other living systems such as marine organisms and fungi. Naturally, they occur as hydrocarbons or in oxygenated forms including lactones, alcohols, acids, aldehydes, and ketones. Sesquiterpenes also include essential oils and aromatic constituents with several pharmacological activities.

“Aroma compounds”, also known as aromas, fragrances, odorants, or flavours—or organoleptic ingredients thereof—are chemical substances with sensorial properties showing a wide variety of odors. They comprise a number of classes of volatile chemical compounds, such as alcohols, aldehydes, ketones, acids, esters, lactones, and terpenes, which are widely used in foods, detergents, cosmetics, and in the pharmaceutical industry. For an individual chemical or class of chemical compounds to impart a smell or fragrance, ideally it must be sufficiently volatile for transmission via the air to the olfactory system in the upper part of the nose. Primarily, the organoleptic properties are important, i.e. the compounds should have advantageous odiferous (olfactory) or gustatory properties. Furthermore, aroma compounds should also have additional positive secondary properties, such as, e.g., an efficient preparation method, the possibility of providing better sensory profiles as a result of synergistic effects with other fragrances, a higher stability under certain application conditions, a higher extendibility, a better higher substantivity, etc. As set forth elsewhere herein ionylideneethane could be identified as an aroma compound, thanks to the present inventors. This finding could not be expected because ionylideneethane was not considered as an aroma compound, thus far. It has further been found by the present inventors that alpha-ionylideneethane can be used for preparing one or more aroma compounds which convey a note of Floral-Violet and/or Woody-Orris/Iris Root to a perfume, fragrance or aroma.

An “aroma compound” as used herein comprises at least one aroma compound, but can comprise also two, three, four, five, six, seven, eight, nine, ten, or even more aroma compounds. It can further comprise other ingredients, such as one or more diluents, or ingredients as defined herein.

Fragrance compositions and ingredients are well known in the art (see, e.g., Fundamentals of Fragrance Chemistry, Charles S. Sell, John Wiley & Sons (2019)) and are also illustrated in the following Examples.

A “perfumed or fragranced product” is a product comprising at least one aroma compound such as alpha-ionylideneethane and/or alpha-ionone, and can encompass, for instance, consumer products such as a fine fragrance, a personal care product, a home care product, and an air care product, preferably wherein the fine fragrance is selected from parfum, extrait de parfum, eau de parfum, millesime, parfum de toilette, eau de toilette, eau de cologne, body splash, after shave, body mists, and baby colognes, preferably wherein the personal care product is selected from lotions, creams, moisturizers, body washes, hand soaps, shampoos, conditioners, and soaps, preferably wherein the home care product is selected from fabric conditioner, fabric softener, laundry detergent, laundry additive, rinse additive, bleach, dryer sheets, perfume beads, car care products, dishwashing detergent, and hard surface cleaners, preferably wherein the air care product is selected from a candle, aerosol, air freshener, liquid electric air freshener, fragrance diffuser, gel air freshener, plug-in air freshener, plug-in oil, and wax melt; see, e.g. EP3468527B1.

The term “protein” or “polypeptide” or “(poly)peptide” or “peptide” (all terms are used interchangeably, if not indicated otherwise) as used herein encompasses isolated and/or purified and/or recombinant (poly)peptides being essentially free of other host cell polypeptides. The term “peptide” as referred to herein comprises at least two, three, four, five, six, seven, eight, nine, ten, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, 100, 150, 200, 250, 300 or even more amino acid residues where the alpha carboxyl group of one is bound to the alpha amino group of another. A post-translational modification of the protein or peptide as used and envisaged herein is the modification of a newly formed protein or peptide and may involve deletion, substitution or addition of amino acids, chemical modification of certain amino acids, for example, amidation, acetylation, phosphorylation, glycosylation, formation of pyroglutamate, oxidation/reduction of sulfa group on a methionine, or addition of similar small molecules, to certain amino acids.

As well known to those skilled in the art, enzymes are proteins. Enzymes bind to their substrates and transform them into products. A plot of the initial reaction velocity versus s substrate concentration depicts a rectangular hyperbola. The reaction velocity (v) equals (Vmax [A])/(Km+[A]) as described by the Michaelis-Menten equation where Vmax is the maximal velocity, [A] is the substrate concentration, and Km is the Michaelis constant, or the substrate concentration at half maximal velocity. Steady-state enzyme kinetics are used to determine the Km value for substrates, the Vmax value for enzymes, and the Ki values for various inhibitors, including drugs.

The “turnover number” of an enzyme (kcat or catalytic rate constant) is the maximal number of molecules of substrate converted to product per active site per unit time of several different substrates to different products. The kcat/Km value, or specificity constant, of the various substrates can be compared. That substrate with the highest value is the best substrate for the enzyme, accounting for the name specificity constant. The rate of any reaction is limited by the rate at which reactant molecules collide. The diffusional limiting rate for a bimolecular reaction is 10⁸ to 10⁹ M⁻¹ s⁻¹. The ratio of kcat/Km is a first-order rate constant. The product of kcat/Km and the substrate concentration (at subsaturating levels) yields the rate of the enzyme-catalyzed reaction. This rate is proportional to the substrate concentration and is therefore designated first order. Enzymes that have ratios of kcat/Km near 10⁸ to 10⁹ M⁻¹ s⁻¹ (close to the maximum allowed by the rate of diffusion) have achieved catalytic perfection. For example, triose phosphate isomerase (EC 5.3.1.1), an enzyme of the glycolytic pathway, is an enzyme that has this attribute. Most enzymes, however, have specificity constants orders of magnitude below this value. Methods for determining the turnover number of an enzyme are well known in the art; see, e.g., https://doi.org/10.1016/B978-0-12-801238-3.05143-6 or Heckmann et al., PNAS Sep. 15, 2020 117 (37) 23182-23190; https://doi.org/10.1073/pnas.2001562117.

Sequence identity, homology or similarity is defined herein as a relationship between two or more amino acid sequences or two or more nucleic acid sequences, as determined by comparing those sequences. Usually, sequence identities or similarities are compared over the whole length of the sequences, but may also be compared only for a part of the sequences aligning with each other. Preferably, the sequence identities or similarities are compared over the whole length of the sequences, herein. In the art, “identity” or “similarity” also means the degree of sequence relatedness between polypeptide sequences or nucleic acid sequences, as the case may be, as determined by the match between such sequences.

Sequence alignments can be generated with a number of software tools, such as:
Needleman and Wunsch algorithm—Needleman, Saul B. & Wunsch, Christian D. (1970). “A general method applicable to the search for similarities in the amino acid sequence of two proteins”. Journal of Molecular Biology 48 (3): 443-453.

This algorithm is, for example, implemented into the “NEEDLE” program, which performs a global alignment of two sequences. The NEEDLE program, is contained within, for example, the European Molecular Biology Open Software Suite (EMBOSS).
EMBOSS—a collection of various programs: The European Molecular Biology Open Software Suite (EMBOSS), Trends in Genetics 16 (6), 276 (2000).
BLOSUM (BLOcks SUbstitution Matrix)—typically generated on the basis of alignments of conserved regions, e.g., of protein domains (Henikoff S, Henikoff J G: Amino acid substitution matrices from protein blocks. Proceedings of the National Academy of Sciences of the USA. 1992 Nov. 15; 89(22): 10915-9). One out of the many BLOSUMs is “BLOSUM62”, which is often the “default” setting for many programs, when aligning protein sequences.
BLAST (Basic Local Alignment Search Tool)—consists of several individual programs (BlastP, BlastN) which are mainly used to search for similar sequence in large sequence databases. BLAST programs also create local alignments. Typically used is the “BLAST” interface provided by NCBI (National Center for Biotechnology Information), which is the improved version (“BLAST2”). The “original” BLAST: Altschul, S. F., Gish, W., Miller, W., Myers, E. W. & Lipman, D. J. (1990) “Basic local alignment search tool.” J. Mol. Biol. 215:403-410; BLAST2: Altschul, Stephen F., Thomas L. Madden, Alejandro A. Schaffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402.

Sequence identity as used herein is preferably the value as determined by the EMBOSS Pairwise Alignment Algorithm “Needle”. In particular, the NEEDLE program from the EMBOSS package can be used (version 2.8.0 or higher, EMBOSS: The European Molecular Biology Open Software Suite—Rice, P., et al. Trends in Genetics (2000) 16: 276-277; http://emboss.bioinformatics.nl) using the NOBRIEF option (‘Brief identity and similarity’ to NO) which calculates the “longest-identity”. The identity, homology or similarity between the two aligned sequences is calculated as follows: Number of corresponding positions in the alignment showing an identical amino acid in both sequences divided by the total length of the alignment after subtraction of the total number of gaps in the alignment. For alignment of amino acid sequences the default parameters are: Matrix=Blosum62; Open Gap Penalty=10.0; Gap Extension Penalty=0.5. For alignment of nucleic acid sequences the default parameters are: Matrix=DNAfull; Open Gap Penalty=10.0; Gap Extension Penalty=0.5.

The term “alpha-ionylideneethane synthase” as used herein means a sesquiterpene synthase which is able to convert farnesyl diphosphate to alpha-ionylideneethane. Accordingly, the term “alpha-ionylideneethane synthase activity” as used herein means an enzymatic activity which catalyses the conversion of farnesyl diphosphate to alpha-ionylideneethane, preferably via cyclization of farnesyl diphosphate to alpha-ionylideneethane. Alpha-ionylideneethane synthase genes have been found in microorganisms, including fungi as well as bacteria, and are well described in the art (Takino et al., J. Am. Chem. Soc. 2018, 140, 39, 12392-12395; Siewers et al., Appl. Environ. Microbiol. 72:4619-4626 (2006); Otto et al., Microb Cell Fact (2019) 18: 205; Inomata et al., Bioscience, Biotechnology, and Biochemistry, Volume 68, Issue 12, 1 Jan. 2004, Pages 2571-2580, https://doi.org/10.1271/bbb.68.2571; Takino et al., 2019, Bioscience Biotechnology and Biochemistry, 83(9), 1642-1649). For instance, Takino et al., 2019, describe the cyclization of farnesyl diphosphate to alpha-ionylideneethane catalyzed by a novel sesquiterpene synthase, BcABA3, which exhibits low amino acid sequence identities with sesquiterpene synthases, in a biosynthetic pathway of abscisic acid in phytopathogenic fungi. Another version of the BcABA3 enzyme and its use in abscisic acid production is reported in the Chinese patent application CN108753744.

“Homologues” means bacterial, fungal, plant or animal homologues of the protein or enzyme referred to herein, such as the alpha-ionylideneethane synthase as defined herein, but also includes truncated sequences, single-stranded DNA or RNA of the coding and non-coding DNA sequence.

Enzyme variants may be defined by their sequence identity when compared to a parent protein or enzyme such as the alpha-ionylideneethane synthases with the amino acid sequences depicted in any one of SEQ ID NO. 1 to 17 or 19 to 33.
Sequence identity usually is provided as “% sequence identity” or “% identity”. To determine the percent-identity between two amino acid sequences in a first step a pairwise sequence alignment is generated between those two sequences, wherein the two sequences are aligned over their complete, entire or full length (i.e., a pairwise global alignment). The alignment is generated with a program or software described herein. The preferred alignment for the purpose of this invention is that alignment, from which the highest sequence identity can be determined.

The term “protein” or “polypeptide” or “peptide” as used herein encompasses peptidomimetics of the protein or enzyme as referred to herein, such as the alpha-ionylideneethane synthase as defined herein. As known in the art, peptidomimetics are compounds whose essential elements (pharmacophore) mimic a natural peptide or protein in 3D space and which retain the ability to interact with the biological target (such as substrate of the enzyme) and produce the same biological effect (for example, alpha-ionylideneethane synthase activity); see, e.g., the review by Vagner et al. 2008, Current Opinion in Chemical Biology 12, Pages 292-296. Peptidomimetics are designed to circumvent some of the problems associated with a natural polypeptide, e.g., stability against proteolysis (duration of biological activity) and poor bioavailability. Certain other properties, such as selectivity for the biological target or substrate or potency of the biological activity, such as the aforementioned biological activity, often can be substantially improved.

Discrepancies between a nucleic acid sequence or an amino acid sequence of a protein or enzyme referred to herein, such as the alpha-ionylideneethane synthase as defined herein, and the nucleic acid sequence or amino acid sequence of a functional homologue of said enzyme, may in particular be the result of modifications performed, e.g., to improve a property of the enzyme or nucleic acid (e.g., improved expression of the enzyme or increased enzymatic activity of the enzyme) by a biological technique known to the skilled person in the art, such as, e.g., molecular evolution or rational design, or by using a mutagenesis technique known in the art and described elsewhere herein (random mutagenesis, site-directed mutagenesis, directed evolution, gene recombination, etc.).

The enzyme's or the nucleic acid's sequence may be altered, as a result of one or more natural occurring variations. Examples of such natural modifications or variations are differences in glycosylation (more broadly defined as “post-translational modifications”), differences due to alternative splicing, and single-nucleic acid polymorphisms (SNPs). The nucleic acid may be modified such that it encodes a polypeptide that differs by at least one amino acid, or two, three, four, five, six, or even more amino acids so that it encodes a polypeptide comprising one or more amino acid substitutions, deletions and/or insertions, which polypeptide still has biological or enzymatic activity, such as alpha-ionylideneethane synthase activity as defined herein. Further, use may be made of artificial gene-synthesis (synthetic DNA), codon optimisation or codon pair optimisation, e.g. based on a method as described in WO 2008/000632 or as offered by commercial DNA synthesizing companies like DNA2.0, Geneart, and GenScript.

The enzyme's or the nucleic acid's sequence may be altered by gene editing. Gene editing or genome editing is a type of genetic engineering in which DNA is inserted, replaced, or removed from a genome and which can be obtained by using a variety of techniques such as “gene shuffling” or “directed evolution” consisting of iterations of DNA shuffling followed by appropriate screening and/or selection to generate variants of nucleic acids or portions thereof encoding proteins having a modified biological activity (Castle et al., (2004) Science 304(5674): 1151-4; U.S. Pat. Nos. 5,811,238 and 6,395,547), or with “T-DNA activation” tagging (Hayashi et al. Science (1992) 1350-1353), where the resulting transgenic organisms show dominant phenotypes due to modified expression of genes close to the introduced promoter, or with “TILLING” (Targeted Induced Local Lesions In Genomes) and refers to a mutagenesis technology useful to generate and/or identify nucleic acids encoding proteins with modified expression and/or activity. TILLING also allows selection of organisms carrying such mutant variants. Methods for TILLING are well known in the art (McCallum et al., (2000) Nat Biotechnol 18: 455-457; reviewed by Stemple (2004) Nat Rev Genet 5(2): 145-50). Another technique uses artificially engineered nucleases like Zinc finger nucleases, Transcription Activator-Like Effector Nucleases (TALENs), the CRISPR/Cas system, and engineered meganuclease such as re-engineered homing endonucleases (Esvelt, KM.; Wang, HH. (2013), Mol Syst Biol 9 (1): 641; Tan, WS. et al. (2012), Adv Genet 80: 37-97; Puchta, H.; Fauser, F. (2013), Int. J. Dev. Biol 57: 629-637).

Derivatives of the protein or enzyme as referred to herein, such as the alpha-ionylideneethane synthase as defined herein, comprise functional, i.e. enzymatically active variants which can be obtained by deletion, insertion, or substitution of amino acid residues from/into the amino acid sequence. A modification or mutation may be a replacement of an amino acid residue by a different one, a deletion of an amino acid residue, or an insertion of an amino acid residue. For instance, amino acid residues that are involved in substrate binding can be modified or mutated. To provide a specific example, the modified or mutated amino acid sequence has preferably improved, e.g., increased alpha-ionylideneethane synthase activity, in comparison to the unmodified amino acid sequence as shown in any one of SEQ ID NO. 1 to 17 or 19 to 33. To this end, for instance, site-directed mutagenesis of said alpha-ionylideneethane synthases can be carried out, focusing on amino acid residues found in highly conserved motifs among homologues to identify mutants producing intermediates of alpha-ionylideneethane and/or alpha-ionone synthesis reaction, or to elucidate the cyclization mechanism in more detail.

Preferably, said homologues, variants, derivatives, or peptidomimetics of the protein or enzyme referred to herein, such as the alpha-ionylideneethane synthase as defined herein, have at least 50%, 60%, 70%, 80%, 90%, or even 100% of the biological or enzymatic activity, of the non-modified or non-mutated protein or enzyme, for example, at least 50%, 60%, 70%, 80%, 90%, or even 100% of the alpha-ionylideneethane synthase activity of the non-modified or non-mutated alpha-ionylideneethane synthase of any one of amino acid sequence of SEQ ID NO. 1 to 17 or 19 to 33. Said homologues, variants, derivatives or peptidomimetics preferably also maintain the substrate specificity and/or substrate preference of the non-modified or non-mutated protein or enzyme, such as the substrate specificity and/or substrate preference of the alpha-ionylideneethane synthase of any one of SEQ ID NO. 1 to 17 or 19 to 33. For instance, the homologue, variant, derivative or peptidomimetic of the alpha-ionylideneethane synthase of any one of SEQ ID NO. 1 to 17 or 19 to 33 is able to convert farnesyl diphosphate to alpha-ionylideneethane, as explained elsewhere herein. In a preferred embodiment the homologues, variants, derivatives or peptidomimetics have a turnover number of at least 90% of the turnover number of the alpha-ionylideneethane synthase of any one of amino acid sequence of SEQ ID NO. 1 to 17 or 19 to 33.

DNA and the proteins that they encode can be modified using various techniques known in molecular biology to generate variant proteins or enzymes with new or altered properties (see, e.g., Sambrook; Ausubel, cited elsewhere herein).

Random PCR mutagenesis is described, e.g., in Rice (1992) Proc. Natl. Acad. Sci. USA 89:5467-5471, and combinatorial multiple cassette mutagenesis is described, e.g., in Crameri (1995) Biotechniques 18:194-196.

Alternatively, nucleic acids, e.g., genes, can be reassembled after random, or “stochastic,” fragmentation, see, e.g., U.S. Pat. Nos. 6,291,242; 6,287,862; 6,287,861; 5,955,358; 5,830,721; 5,824,514; 5,811,238; 5,605,793.

Alternatively, modifications, additions or deletions are introduced by error-prone PCR, shuffling, site-directed mutagenesis, assembly PCR, sexual PCR mutagenesis, in vivo mutagenesis (phage-assisted continuous evolution, in vivo continuous evolution), cassette mutagenesis, recursive ensemble mutagenesis, exponential ensemble mutagenesis, site-specific mutagenesis, gene reassembly, gene site saturation mutagenesis (GSSM), synthetic ligation reassembly (SLR), recombination, recursive sequence recombination, phosphothioate-modified DNA mutagenesis, uracil-containing template mutagenesis, gapped duplex mutagenesis, point mismatch repair mutagenesis, repair-deficient host strain mutagenesis, chemical mutagenesis, radiogenic mutagenesis, deletion mutagenesis, restriction-selection mutagenesis, restriction-purification mutagenesis, artificial gene synthesis, ensemble mutagenesis, chimeric nucleic acid multimer creation, and/or a combination of these and other methods.

Alternatively, “gene site saturation mutagenesis” or “GSSM” includes a method that uses degenerate oligonucleotide primers to introduce point mutations into a polynucleotide, as described in detail, in U.S. Pat. Nos. 6,171,820 and 6,764,835.

Alternatively, Synthetic Ligation Reassembly (SLR) includes methods of ligating oligonucleotide building blocks together non-stochastically, as disclosed in, e.g., U.S. Pat. No. 6,537,776.

Alternatively, Tailored multi-site combinatorial assembly (“TMSCA”) is a method of producing a plurality of progeny polynucleotides having different combinations of various mutations at multiple sites by using at least two mutagenic non-overlapping oligonucleotide primers in a single reaction. Said method is described, e.g., in WO 2009/018449.

The protein or enzyme as referred to herein, such as the alpha-ionylideneethane synthase as defined herein, can also be a fusion protein. The term “fusion protein” as used herein denotes a chimeric protein (literally, made of parts from different sources) which is created through the joining of two or more genes that originally coded for separate proteins. Translation of this fusion gene results in a single or multiple polypeptides with functional properties derived from each of the original proteins. For example, the fusion protein as defined herein can comprise an affinity tag for protein purification (such as His tag, FLAG tag etc., see, e.g. Kimple et al., 2015, Curr Protoc Protein Sci.; 73: Unit-9.9. doi:10.1002/0471140864.ps0909s73.), or a label for detection. A “label” as referred to herein is a detectable compound or composition that is conjugated directly or indirectly to another molecule, such as the alpha-ionylideneethane synthase as defined herein, to facilitate detection of that molecule. Specific, non-limiting examples of labels include fluorescent tags, enzymatic linkages, and radioactive isotopes well known in the art. In one embodiment, a protease cleavage site and/or linker (i.e. a protease cleavage site; or a linker; or both a protease cleavage site and a linker; or the linker comprises a protease cleavage site) can be present between the protein or enzyme as referred to herein, such as the alpha-ionylideneethane synthase as defined herein, and label or purification tag. For instance, the protease cleavage site can be used to cleave off the purification tag by treatment with proteases, such as enterokinase or thrombin, if desired. For example, a His tag can be used as a tag for expression and purification while the protein or enzyme as referred to herein, such as the alpha-ionylideneethane synthase as defined herein, can be isolated post-cleavage with the protease. As well known by the skilled person, besides the basic role in linking the functional domains together (as in flexible and rigid linkers), linkers may offer many other advantages for the production of fusion proteins, such as improving biological activity, increasing expression yield, and achieving desirable pharmacokinetic profiles. The linker can be, e.g., a protein/peptide linker such as a polyglycine linker or other linker known in the art (see, e.g., Chen et al., Adv Drug Deliv Rev. 2013; 65(10): 1357-1369). Evidently, the linker can be designed in a way that it comprises a protease cleavage site. In another aspect, the fusion protein can carry a signal peptide for targeting the expressed polypeptide, e.g. to a specific organelle, as explained elsewhere herein.

The fusion protein as defined herein can be manufactured by chemical synthesis or recombinant molecular biology techniques well known to the person skilled in the art. This applies mutatis mutandis to the isolation of fusion protein from the host cell or supernatant; see, e.g., Sambrook et al., Molecular cloning: a laboratory manual/Sambrook, Joseph; Russell, David W. —. 3rd ed. —New York: Cold Spring Harbor Laboratory, 2001; Ausubel, Current Protocols in Molecular Biology, Green Publishing Associates and Wiley Interscience, N.Y. (1994).

The term “nucleic acid” as used herein, includes reference to a deoxyribonucleotide or ribonucleotide polymer, i.e. a polynucleotide, in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids). A polynucleotide can be full-length or a sub-sequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are “polynucleotides” as the term is used herein. It will be appreciated that a great variety of modifications have been made to DNA and RNA that serve many useful purposes known to those of skill in the art. The term “polynucleotide” as it is employed herein embraces such chemically, enzymatically or metabolically modified forms of polynucleotides, as well as the chemical forms of DNA and RNA characteristic of viruses and cells, including among other things, simple and complex cells. Every nucleic acid sequence herein that encodes a polypeptide or enzyme such as the alpha-ionylideneethane synthase as defined herein also, by reference to the genetic code, describes every possible silent variation of the nucleic acid. The term “conservatively modified variants” applies to both amino acid and nucleic acid sequences. With respect to particular nucleic acid sequences, the term “conservatively modified variants” refers to those nucleic acids which encode identical or conservatively modified variants of the amino acid sequences due to the degeneracy of the genetic code. The term “degeneracy of the genetic code” refers to the fact that a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. The terms “polypeptide”, “peptide” and “protein” are used interchangeably herein to refer to a polymer of amino acid residues. An “enzymatically active fragment of the amino acid sequence” of the protein or enzyme as referred to herein, such as the alpha-ionylideneethane synthase as defined herein, means a stretch of at least 30, 35, 40, 45, 50, 60, 70, 80, 90, 100, 150, 200, or 250 amino acid residues having biological or enzymatic activity as referred to herein, such as alpha-ionylideneethane synthase activity as defined herein. The terms “polypeptide”, “peptide” and “protein” apply also to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein, that protein is specifically reactive to antibodies elicited to the same protein but consisting entirely of naturally occurring amino acids. The terms “polypeptide”, “peptide” and “protein” are also inclusive of modifications including, but not limited to, glycosylation, lipid attachment, sulphation, gamma-carboxylation of glutamic acid residues, hydroxylation and ADP-ribosylation. Within the context of the present application, oligomers (such as oligonucleotides, oligopeptides) are considered a species of the group of polymers. Oligomers have a relatively low number of monomeric units, in general 2-100, in particular 6-100, including, e.g., primer sequences, such as used for cloning of the alpha-ionylideneethane synthase, as used in the Examples.

The term “heterologous” when used with respect to a nucleic acid (DNA or RNA) or protein or enzyme of the disclosure, such as an alpha-ionylideneethane synthase as defined herein, refers to a nucleic acid or protein that does not occur naturally as part of the organism, cell, genome or DNA or RNA sequence in which it is present, or that is found in a cell or location or locations in the genome or DNA or RNA sequence that differ from that in which it is found in nature. Heterologous nucleic acids or proteins or enzymes of the disclosure, such as an alpha-ionylideneethane synthase as defined herein, are not endogenous to the cell into which they are introduced, but have been obtained from another cell or synthetically or recombinantly produced. Generally, though not necessarily, such nucleic acids encode proteins that are not normally produced by the cell in which the DNA is expressed. A gene that is endogenous to a particular host cell but has been modified from its natural form, through, for example, the use of DNA shuffling, is also called heterologous. The term “heterologous” also includes non-naturally occurring multiple copies of a naturally occurring DNA sequence. Thus, the term “heterologous” may refer to a DNA segment that is foreign or heterologous to the cell, or homologous to the cell but in a position and/or a number within the host cell nucleic acid in which the segment is not ordinarily found. Exogenous DNA segments are expressed to yield exogenous polypeptides.

A “homologous” DNA sequence as used herein is a DNA sequence that is naturally associated with a host cell into which it is introduced. Any nucleic acid or protein that one of skill in the art would recognize as heterologous or foreign to the cell in which it is expressed is herein encompassed by the term heterologous nucleic acid or protein.

The terms “modified”, “modification”, “mutated”, or “mutation”, as used herein regarding proteins or polypeptides compared to another protein or polypeptide (for example, compared to the alpha-ionylideneethane synthase as defined herein comprising or consisting of the amino acid sequences of Any of SEQ ID NO. 1 to 17 or 19 to 33) apply mutatis mutandis to nucleotide or nucleic acid sequences. The mentioned terms are used to indicate that the modified nucleotide or nucleic acid sequences encoding the protein or polypeptide having biological or enzymatic activity such as alpha-ionylideneethane synthase activity has at least one difference in the nucleotide or nucleic acid sequence compared to the nucleotide or nucleic acid sequence of the protein or polypeptide with which it is compared, e.g., the amino acid sequence of any one of Any of SEQ ID NO. 1 to 17 or 19 to 33. The terms are used irrespective of whether the modified or mutated protein actually has been obtained by mutagenesis of nucleic acids encoding these amino acids or modification of the polypeptide or protein, or in another manner, e.g. using artificial gene-synthesis methodology. Mutagenesis is a well-known method in the art, and includes, for example, site-directed mutagenesis by means of PCR or via oligonucleotide-mediated mutagenesis, as described in Sambrook, J., and Russell, D. W. Molecular Cloning: A Laboratory Manual.3d ed., Cold Spring Harbor Laboratory Press, Cold Spring Harbor, NY, (2001). The term “modified”, “modification”, “mutated”, or “mutation” as used herein regarding genes is used to indicate that at least one nucleotide in the nucleotide sequence of that gene or a regulatory sequence thereof, is different from the nucleotide sequence that it is compared with, e.g. a nucleotide sequence encoding the amino acid sequences of Any of SEQ ID NO. 1 to 17 or 19 to 33. A modification or mutation may in particular be a replacement of a nucleotide by a different one, a deletion of a nucleotide or an insertion of a nucleotide.

The nucleic acid encoding the protein or enzyme referred to herein, such as the alpha-ionylideneethane synthase as defined herein, is operatively linked to expression control sequences allowing expression in prokaryotic or eukaryotic host cells, or isolated fractions thereof, in a vector or gene construct. Thus, in an aspect, the vector is an expression vector. Expression of the nucleic acid encoding the protein or enzyme referred to herein, such as the alpha-ionylideneethane synthase as defined herein, comprises transcription of the polynucleotide into a translatable mRNA. Regulatory elements ensuring expression in prokaryotic or eukaryotic host cells are well known in the art. In an aspect, they comprise regulatory sequences ensuring initiation of transcription and/or poly-A signals ensuring termination of transcription and stabilization of the transcript. Additional regulatory elements may include transcriptional as well as translational enhancers. Possible regulatory elements permitting expression in prokaryotic host cells comprise, e.g., the lac-, trp- or tac-promoter in E. coli, or Rhodobacter promoters (https://doi.org/10.1073/pnas.2010087117), and examples for regulatory elements permitting expression in eukaryotic host cells are the AOX1- or the GAL1-promoter in yeast or the CMV-, SV40-, RSV-promoter (Rous sarcoma virus), CMV-enhancer, SV40-enhancer or a globin intron in mammalian and other animal cells. Plant promoters are described, e.g., in Plant Biotechnology: Principles and Applications, pp 117-172, 2017. Moreover, inducible expression control sequences may be used in an expression vector. Such inducible vectors may comprise tet or lac operator sequences or sequences inducible by heat shock or other environmental factors. Suitable expression control sequences are well known in the art. Beside elements which are responsible for the initiation of transcription such regulatory elements may also comprise transcription termination signals, such as the SV40-poly-A site or the tk-poly-A site, downstream of the polynucleotide. In this context, suitable expression vectors are known in the art, such as Okayama-Berg cDNA expression vector pcDV1 (Pharmacia), pBluescript (Stratagene), pCDM8, pRc/CMV, pcDNA1, pcDNA3 (Invitrogen) or pSPORT1 (Invitrogen). Expression vectors derived from viruses such as retroviruses, vaccinia virus, adeno-associated virus, herpes viruses, or bovine papilloma virus, may be used for delivery of the polynucleotide or vector into a targeted cell population.

Methods which are well known to those skilled in the art can be used to construct vectors or gene constructs comprising the nucleic acid encoding the protein or enzyme referred to herein, such as the alpha-ionylideneethane synthase as defined herein; see, for example, the techniques described in Sambrook, Molecular Cloning A Laboratory Manual, Cold Spring Harbor Laboratory (2001) N.Y. and Ausubel, Current Protocols in Molecular Biology, Green Publishing Associates and Wiley Interscience, N.Y. (1994).

The term “gene” as used herein is used broadly to refer to any segment of nucleic acid associated with a biological function, such as the nucleic acid encoding the enzymatically active alpha-ionylideneethane synthase as defined herein. Thus, genes include coding sequences and/or the regulatory sequences required for their expression. For example, gene refers to a nucleic acid fragment that expresses mRNA or functional RNA, or encodes a specific protein, and which includes regulatory sequences. Genes also include non-expressed DNA segments that, for example, form recognition sequences for other proteins. Genes can be obtained from a variety of sources, including cloning from a source of interest or synthesizing from known or predicted sequence information, and may include sequences designed to have desired parameters.

The term “chimeric gene” as used herein refers to any gene that contains 1) DNA sequences, including regulatory and coding sequences that are not found together in nature, or 2) sequences encoding parts of proteins not naturally adjoined, or 3) parts of promoters that are not naturally adjoined. Accordingly, a chimeric gene may comprise regulatory sequences and coding sequences that are derived from different sources, or comprise regulatory sequences and coding sequences derived from the same source, but arranged in a manner different from that found in nature.

A “gene construct” as used herein can vary in complexity according to the insertion of interest. The construct can be designed to be inserted randomly into the genome of an organism, which is called transgenesis by addition, or can be designed to be inserted into the genome at a specific targeted site, into the correct position of a determined chromosome, which is called transgenesis by homologous recombination. In both cases, the construct must be impeccable, with structures to control gene expression, such as a promoter, a site of transcription initiation, a site of polyadenylation, and a site of transcription termination. That is, the information which is being inserted into the receptor genome has a beginning, middle, and an end, thus avoiding problems of uncontrolled expression in the host cell or organism.

The terms “open reading frame” and “ORF” as used herein refer to the amino acid sequence encoded between translation initiation and termination codons of a coding sequence. The terms “initiation codon” and “termination codon” refer to a unit of three adjacent nucleotides (‘codon’) in a coding sequence that specifies initiation and chain termination, respectively, of protein synthesis (mRNA translation).

“Coding sequence” as used herein refers to a DNA or RNA sequence that codes for a specific amino acid sequence and excludes the non-coding sequences. It may constitute an “uninterrupted coding sequence”, i.e. lacking an intron, such as in a cDNA or it may include one or more introns bound by appropriate splice junctions. An “intron” is a sequence of RNA which is contained in the primary transcript but which is removed through cleavage and re-ligation of the RNA within the cell to create the mature mRNA that can be translated into a protein.

“Regulatory sequences” as used herein refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences which may be a combination of synthetic and natural sequences. As is noted above, the term “suitable regulatory sequences” is not limited to promoters. Examples of regulatory sequences include promoters (such as transcriptional promoters, constitutive promoters, inducible promoters), operators, enhancers, mRNA ribosomal binding sites, and appropriate sequences which control transcription and translation initiation and termination. Nucleic acid sequences are “operably linked” when the regulatory sequence functionally relates to the DNA or cDNA sequence of the disclosure. As used herein, the term “operably linked” or “operatively linked” refers to a juxtaposition wherein the components so described are in a relationship permitting them to function in their intended manner. A control sequence “operably linked” to another control sequence and/or to a coding sequence is ligated in such a way that transcription and/or expression of the coding sequence is achieved under conditions compatible with the control sequence. Generally, operably linked means that the nucleic acid sequences being linked are contiguous and, where necessary to join two protein coding regions, contiguous and in the same reading frame. Each of the regulatory sequences may independently be selected from heterologous and homologous regulatory sequences.

“Promoter” as used herein refers to a nucleotide sequence, usually upstream (5′) to its coding sequence, which controls the expression of said coding sequence by providing the recognition for RNA polymerase and other factors required for proper transcription. “Promoter” includes a minimal promoter that is a short DNA sequence comprised of a TATA box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence which can stimulate promoter activity and may be an innate element of the promoter or a heterologous element inserted to enhance the level or tissue specificity of a promoter. It is capable of operating in both orientations (normal or flipped), and is capable of functioning even when moved either upstream or downstream from the promoter. Both enhancers and other upstream promoter elements bind sequence-specific DNA-binding proteins that mediate their effects. Promoters may be derived in their entirety from a native gene, or be composed of different elements derived from different promoters found in nature, or even be comprised of synthetic DNA segments. A promoter may also contain DNA sequences that are involved in the binding of protein factors which control the effectiveness of transcription initiation in response to physiological or developmental conditions.

“Expression cassette” as used herein means a DNA sequence capable of directing expression of a particular nucleotide sequence, for example, a nucleotide sequence encoding the alpha-ionylideneethane synthase as defined herein, in an appropriate host cell as defined herein, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example, antisense RNA or a non-translated RNA, in the sense or antisense direction. The expression cassette comprising the nucleotide sequence of interest may be chimeric, meaning that at least one of its components is heterologous with respect to at least one of its other components. The expression cassette may also be one which is naturally occurring but has been obtained in a recombinant form useful for heterologous expression. The expression of the nucleotide sequence in the expression cassette may be under the control of a constitutive promoter or of an inducible promoter which initiates transcription only when the host cell is exposed to some particular external stimulus. In the case of a multicellular organism, the promoter can also be specific to a particular tissue or organ or stage of development, e.g., in plant development.

The term “vector” as used herein refers to a construction comprised of genetic material designed to direct transformation of a targeted cell. A vector contains multiple genetic elements oriented positionally and sequentially, i.e., operatively linked with other necessary elements such that the nucleic acid in a nucleic acid cassette can be transcribed and when necessary, translated in the transformed cells. In particular, the vector may be selected from the group of viral vectors, (bacterio)phages, cosmids or plasmids. The vector may also be a yeast artificial chromosome (YAC), a bacterial artificial chromosome (BAC) or Agrobacterium binary vector. The vector may be in double or single stranded linear or circular form which may or may not be self transmissible or mobilizable, and which can transform host organisms such as, e.g., Rhodobacter, either by integration into the cellular genome or exist extrachromosomally (e.g., autonomous replicating plasmid with an origin of replication). Specifically included are shuttle vectors by which means a DNA vehicle capable, naturally or by design, of replication in two different host organisms as defined herein. Preferably, the nucleic acid in the vector is under the control of, and operably linked to, an appropriate promoter or other regulatory elements for transcription in a host cell as specified herein. The vector may be a bi-functional expression vector which functions in multiple hosts. In the case of genomic DNA, this may contain its own promoter or other regulatory elements, and in the case of cDNA this may be under the control of an appropriate promoter or other regulatory elements for expression in the host cell. Vectors containing a nucleic acid can be prepared based on methodology known in the art. For instance, use can be made of a cDNA sequence encoding the alpha-ionylideneethane synthase as defined herein operably linked to suitable regulatory elements, such as transcriptional or translational regulatory nucleic acid sequences.

The term “vector” as used herein, includes reference to a vector for standard cloning work (“cloning vector”) as well as to more specialized type of vectors, like an (autosomal) expression vector and a cloning vector used for integration into the chromosome of the host cell (“integration vector”).

“Cloning vectors” typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector.

The term “expression vector” as used herein refers to a DNA molecule, linear or circular, that comprises a segment encoding a polypeptide of interest under the control of (, i.e. operably linked to) additional nucleic acid segments that provide for its transcription. Such additional segments may include promoter and terminator sequences, and may optionally include one or more origins of replication, one or more selectable markers, an enhancer, a polyadenylation signal, and the like. Expression vectors are generally derived from plasmid or viral DNA, or may contain elements of both. In particular, an expression vector comprises a nucleotide sequence that comprises in the 5′ to 3′ direction and operably linked: (a) a transcription and translation initiation region that are recognized by the host organism, (b) a coding sequence for a polypeptide of interest, and (c) a transcription and translation termination region that are recognized by the host organism. “Plasmid” refers to autonomously replicating extrachromosomal DNA which is not integrated into a microorganism's genome and is usually circular in nature.

An “integration vector” refers to a DNA molecule, linear or circular, that can be incorporated, e.g., into a microorganism's genome, such as a bacteria's genome, and provides for stable inheritance of a gene encoding a polypeptide of interest, such as the alpha-ionylideneethane synthase as defined herein. The integration vector generally comprises one or more segments comprising a gene sequence encoding a polypeptide of interest under the control of (i.e., operably linked to) additional nucleic acid segments that provide for its transcription.

Such additional segments may include promoter and terminator sequences, and one or more segments that drive the incorporation of the gene of interest into the genome of the target cell, usually by the process of homologous recombination. Typically, the integration vector will be one which can be transferred into the target cell, but which has a replicon which is non-functional in that organism. Integration of the segment comprising the gene of interest may be selected if an appropriate marker is included within that segment. One or more nucleic acid sequences encoding appropriate signal peptides that are not naturally associated with a polypeptide to be expressed in a host cell as defined herein, preferably a host cell of the invention, can be incorporated into (expression) vectors. For example, a DNA sequence for a signal peptide leader can be fused in-frame to a nucleic acid of the disclosure so that the protein or enzyme referred to herein, such as the alpha-ionylideneethane synthase as defined herein, is initially translated as a fusion protein comprising the signal peptide. Depending on the nature of the signal peptide, the expressed polypeptide will be targeted differently. A secretory signal peptide that is functional in the intended host cells, for instance, enhances extracellular secretion of the expressed polypeptide. Other signal peptides direct the expressed polypeptide to certain organelles, like the chloroplasts, mitochondria and peroxisomes. The signal peptide can be cleaved from the polypeptide upon transportation to the intended organelle or from the cell. It is possible to provide a fusion of an additional peptide sequence at the amino or carboxyl terminal end of the polypeptide.

The host cell is transformed with the vector or gene construct as disclosed herein. The skilled artisan is well aware of the genetic elements that must be present on the genetic construct to successfully transform, select and propagate host cells containing the vector or gene construct as disclosed herein. The host cell is capable of expressing a polypeptide or enzyme as referred to herein, such as a protein with alpha-ionylideneethane synthase activity, included in the vector or gene construct of the disclosure. The host cell also comprises farnesyl diphosphate as a substrate for the expressed, enzymatically active alpha-ionylideneethane synthase.

“Transformation” and “transforming”, as used herein, refers to the introduction of a heterologous nucleotide sequence, such as the nucleotide sequence encoding a protein or enzyme as referred to herein, such as the alpha-ionylideneethane synthase as defined herein, into a host cell, irrespective of the method used for the insertion, for example, direct uptake, transduction, conjugation, f-mating or electroporation. The exogenous polynucleotide may be maintained as a non-integrated vector, for example, a plasmid, or alternatively, may be integrated into the host cell genome.

A host cell according to the disclosure may be produced based on standard genetic and molecular biology techniques that are generally known in the art, e.g., as described in Sambrook, J., and Russell, D. W. “Molecular Cloning: A Laboratory Manual” 3d ed., Cold Spring Harbor Laboratory Press, Cold Spring Harbor, NY, (2001); and F. M. Ausubel et al, eds., “Current protocols in molecular biology”, John Wiley and Sons, Inc., New York (1987), and later supplements thereto.

The host cell can be any cell selected from a microbial cell, e.g., a bacterial cell, a archaeal cell, a fungal cell, such as a yeast cell, and a protist cell. The host cell can also be an algal cell or a cyanobacterial cell, a non-human animal cell or a mammalian cell, or a plant cell.

Specifically, the host cell can be selected from any one of the following organisms:

### Bacteria

The bacterial host cell can, for example, be selected from the group consisting of the genera Escherichia, Klebsiella, Helicobacter, Bacillus, Lactobacillus, Streptococcus, Amycolatopsis, Rhodobacter, Pseudomonas, Paracoccus or Lactococcus.

gram positive: Bacillus, Streptomyces

Useful gram positive bacterial host cells include, but are not limited to, a Bacillus cell, e.g., Bacillus alkalophius, Bacillus amyloliquefaciens, Bacillus brevis, Bacillus circulans, Bacillus clausii, Bacillus coagulans, Bacillus firmus, Bacillus Jautus, Bacillus lentus, Bacillus licheniformis, Bacillus megaterium, Bacillus pumilus, Bacillus stearothermophilus, Bacillus subtilis, and Bacillus thuringiensis. Most preferred, the prokaryote is a Bacillus cell, preferably, a Bacillus cell of Bacillus subtilis, Bacillus pumilus, Bacillus licheniformis, or Bacillus lentus.

Some other preferred bacteria include strains of the order Actinomycetales, preferably, Streptomyces, preferably Streptomyces spheroides (ATTC 23965), Streptomyces thermoviolaceus (IFO 12382), Streptomyces lividans or Streptomyces murinus or Streptoverticillum verticillium ssp. verticillium. Other preferred bacteria include Rhodobacter sphaeroides, Rhodomonas palustri, Streptococcus lactis. Further preferred bacteria include strains belonging to Myxococcus, e.g., M. virescens.

gram negative: E. coli, Pseudomonas, Rhodobacter, Paracoccus

Preferred gram negative bacteria are Escherichia coli, Pseudomonas sp., preferably, Pseudomonas purrocinia (ATCC 15958) or Pseudomonas fluorescens (NRRL B-11), Rhodobacter capsulatus or Rhodobacter sphaeroides, Paracoccus carotinifaciens or Paracoccus zeaxanthinifaciens).

### Fungi

Aspergillus, Fusarium, Trichoderma

The host cell may be a fungal cell. “Fungi” as used herein includes the phyla Ascomycota, Basidiomycota, Chytridiomycota, and Zygomycota as well as the Oomycota and Deuteromycotina and all mitosporic fungi. Representative groups of Ascomycota include, e.g., Neurospora, Eupenicillium (=Penicillium), Emericella (=Aspergillus), Eurotium (=Aspergillus), and the true yeasts listed below. Examples of Basidiomycota include mushrooms, rusts, and smuts. Representative groups of Chytridiomycota include, e.g., Allomyces, Blastocladiella, Coelomomyces, and aquatic fungi. Representative groups of Oomycota include, e.g. Saprolegniomycetous aquatic fungi (water molds) such as Achlya. Examples of mitosporic fungi include Aspergillus, Penicillium, Candida, and Alternaria. Representative groups of Zygomycota include, e.g., Rhizopus and Mucor.

Some preferred fungi include strains belonging to the subdivision Deuteromycotina, class Hyphomycetes, e.g., Fusarium, Humicola, Tricoderma, Myrothecium, Verticillum, Arthromyces, Caldariomyces, Ulocladium, Embellisia, Cladosporium or Dreschlera, in particular Fusarium oxysporum (DSM 2672), Humicola insolens, Trichoderma resii, Myrothecium verrucana (IFO 6113), Verticillum alboatrum, Verticillum dahlie, Arthromyces ramosus (FERM P-7754), Caldariomyces fumago, Ulocladium chartarum, Embellisia alli or Dreschlera halodes.

Other preferred fungi include strains belonging to the subdivision Basidiomycotina, class Basidiomycetes, e.g. Coprinus, Phanerochaete, Coriolus or Trametes, in particular Coprinus cinereus f. microsporus (IFO 8371), Coprinus macrorhizus, Phanerochaete chrysosporium (e.g. NA-12) or Trametes (previously called Polyporus), e.g. T. versicolor (e.g. PR4 28-A). Further preferred fungi include strains belonging to the subdivision Zygomycotina, class Mycoraceae, e.g. Rhizopus or Mucor, in particular Mucor hiemalis.

### Yeast

Pichia

Saccharomyces

The fungal host cell may be a yeast cell. Yeast as used herein includes ascosporogenous yeast (Endomycetales), basidiosporogenous yeast, and yeast belonging to the Fungi Imperfecti (Blastomycetes). The ascosporogenous yeasts are divided into the families Spermophthoraceae and Saccharomycetaceae. The latter is comprised of four subfamilies, Schizosaccharomycoideae (e.g., genus Schizosaccharomyces), Nadsonioideae, Lipomycoideae, and Saccharomycoideae (e.g. genera Kluyveromyces, Pichia, and Saccharomyces). The basidiosporogenous yeasts include the genera Leucosporidim, Rhodosporidium, Sporidiobolus, Filobasidium, and Filobasidiella. Yeasts belonging to the Fungi Imperfecti are divided into two families, Sporobolomycetaceae (e.g., genera Sporobolomyces and Bullera) and Cryptococcaceae (e.g. genus Candida).

### Eukaryotes

Eukaryotic host cells further include, without limitation, a non-human animal cell, a non-human mammal cell, an avian cell, reptilian cell, insect cell, or a plant cell.

In a preferred embodiment, the host cell is a host cell selected from:
a) a bacterial cell of the group of Gram negative bacteria, such as Rhodobacter (e.g. Rhodobacter sphaeroides or Rhodobacter capsulatus), Paracoccus (e.g. P. carotinifaciens, P. zeaxanthinifaciens), Escherichia or Pseudomonas;
b) a bacterial cell selected from the group of Gram positive bacteria, such as Bacillus, Corynebacterium, Brevibacterium, Amycolatopis;
c) a fungal cell selected from the group of Aspergillus, Blakeslea, Peniciliium, Phaffia (Xanthophyllomyces), Pichia, Saccharamoyces, Kluyveromyces, Yarrowia, and Hansenula;
d) a transgenic plant cell or a culture comprising transgenic plant cells, wherein the cell is of a transgenic plant selected from Arabidopsis spp., Nicotiana spp, Cichorum intybus, lacuca sativa, Mentha spp, Artemisia annua, tuber forming plants, oil crops, e.g. Brassica spp. or Brassica napus, flowering plants (angiosperms) which produce fruits, and trees; or
e) a transgenic mushroom or culture comprising transgenic mushroom cells, wherein the microorganism is selected from Schizophyllum, Agaricus and Pleurotisi.

More preferred host cells from organisms are host cells from microorganisms belonging to the genus Escherichia, Saccharomyces, Pichia, Rhodobacter, Pseudomonas or Paracoccus, (e.g. Paracoccus carotinifaciens, Paracoccus zeaxanthinifaciens) and even more preferred those of the species E. coli, S. cerevisae, Rhodobacter sphaeroides, Rhodobacter capsulatus, or Amycolatopis sp.

Particularly preferred is a Rhodobacter host cell selected from the group of Rhodobacter capsulatus and Rhodobacter sphaeroides.

The present invention also relates to a fermentation composition comprising:
(a) a genetically modified microbial host cell cultured in a culture medium, wherein the microbial host cell is a microbial host cell of the invention; and
(b) alpha-ionylideneethane and/or alpha-ionone produced from the microbial host cell of the invention.

The present invention further provides a host cell for preparing alpha-ionone, wherein the host cell comprises farnesyl diphosphate and a heterologous nucleic acid encoding an alpha-ionylideneethane synthase.

The definitions and explanations as regards the term “host cell”, the “host cell (as) disclosed herein” or “the host cell (as) referred to herein” apply mutatis mutandis to the host cell of the invention.

The host cell of the invention comprises a heterologous nucleic acid encoding an alpha-ionylideneethane synthase as disclosed herein, and farnesyl diphosphate as a substrate for the alpha-ionylideneethane synthase.

Advantageously, the host cell of the invention can be used for the production of alpha-ionylideneethane, as demonstrated, in the following Examples. Preferably, the alpha-ionylideneethane is E,Z-alpha-ionylideneethane (1,5,5-trimethyl-6-[(1E,3Z)-3-methyl-penta-1,3-dienyl]cyclohexene).

The host cell of the invention can further be used for the production of alpha-ionone, as shown, in the following Examples. The host cell of the invention is suitable to convert alpha-ionylideneethane to alpha-ionone. Preferably, alpha-ionone is R-alpha-ionone.

Preferably, the alpha-ionylideneethane and/or alpha-ionone is used as a precursor of vitamin A and/or for synthesis of vitamin A, in the host cell of the invention. So said host cell is capable of converting alpha-ionylideneethane to vitamin A.

The host cell can be also used for heterologous reconstitution of a terpene or terpenoid.

The host cell can further be utilized for producing an industrial product, preferably an aroma composition, flavour or fragrance, animal feed, a human nutritional product, a cosmetic, a colorant (carotenoid) or a radical scavenger.

The host cell of the invention can serve as a fermentative production system for producing a sesquiterpene, as defined herein.

In a preferred embodiment of the host cell of the invention, the alpha-ionylideneethane synthase converts farnesyl diphosphate to alpha-ionylideneethane.

In another preferred embodiment of the host cell of the invention, at least part of the produced alpha-ionylideneethane is converted to alpha-ionone by oxidative cleavage chemically or enzymatically.

In a still further preferred embodiment of the invention, the alpha-ionylideneethane synthase is a fungal or bacterial alpha-ionylideneethane synthase. In a preferred embodiment, the alpha-ionylideneethane synthase is from a fungus of the Ascomyta, preferably the Pezizomycotina. The fungus in one embodiment is from the family of the Sclerotiniaceae or the Rutstroemiaceae, for example, a Botrytis species or a Rutstroemia species.

In a further preferred embodiment of the host cell of the invention, the alpha-ionylideneethane synthase comprises an amino acid sequence selected from the group consisting of:
a) an amino acid sequence as shown in any one of Any of SEQ ID NO. 1 to 17 or 19 to 33;
b) an amino acid sequence having at least 40%, 50%, 55%, 60%, 65%, 66%, 70%, 71%, 75%, 80%, 81%, 85%, 86%, 90%, or 95% sequence identity at the amino acid level with any one of Any of SEQ ID NO. 1 to 17 or 19 to 33, having alpha-ionylideneethane synthase activity; and
c) an enzymatically active fragment of the amino acid sequence of a) or b), having alpha-ionylideneethane synthase activity.

In still a further preferred embodiment of the host cell of the invention, the host cell further comprises
(i) one or more nucleic acids encoding (an) enzyme(s) of the mevalonate pathway and/or one or more nucleic acids encoding (an) enzyme(s) of the deoxyxylulose phosphate (DXP) pathway; and/or
(ii) one or more nucleic acids encoding (an) oxidative enzyme(s), preferably one or more nucleic acids encoding a carotene dioxygenase and/or a peroxidase; and/or
(iii) one or more nucleic acids encoding (an) enzyme(s) for synthesis of vitamin A.

In another preferred embodiment of the host cell of the invention, the host cell is a bacterial cell, a yeast cell, a fungal cell, an algal cell or a cyanobacterial cell, a non-human animal cell or a non-human mammalian cell, a non-vertebrate cell or a plant cell, preferably a bacterial cell, or a yeast cell. In one embodiment the host cell is an isolated cell, i.e. it is not within the context of a multicellular organism. More preferably, the host cell is a Saccharomyces cerevisiae host cell, or a Rhodobacter host cell, even more preferably, a Rhodobacter sphaeroides host cell.

In addition, the invention relates to a composition comprising (i) the host cell of the invention, alpha-ionylideneethane and/or alpha-ionone, or (ii) the alpha-ionylideneethane synthase as defined herein, alpha-ionylideneethane and/or alpha-ionone. Preferably, alpha-ionylideneethane and/or alpha-ionone is produced by the methods of the invention.

The invention also pertains to a kit comprising the host cell of the invention, or the aroma compound or composition of the invention.

Further, the present invention is directed to a method for preparing alpha-ionone, the method comprising converting farnesyl diphosphate, into alpha-ionylideneethane, in the presence of an enzyme, the enzyme comprising a first segment comprising a tag peptide and a second segment comprising an alpha-ionylideneethane synthase, preferably an alpha-ionylideneethane synthase comprises an amino acid sequence having at least 50%, 55%, 60%, 65%, 66%, 70%, 71%, 75%, 76%, 80%, 81%, 85%, 86%, 90%, 91%, 92%, 93%, 94%, 95%, 96%, 97%, 98%, 99% or even 100% sequence identity at the amino acid level with any of SEQ ID NO: 1 to 17 or 19 to 33, preferably with SEQ ID NO: 1. An enzyme comprising said first and said second segment may herein be referred to as a ‘tagged enzyme’.

Also, the present invention is directed to the use of such a tagged enzyme version of an alpha-ionylideneethane synthase having at least 50%, 55%, 60%, 65%, 66%, 70%, 71%, 75%, 76%, 80%, 81%, 85%, 86%, 90%, 91%, 92%, 93%, 94%, 95%, 96%, 97%, 98%, 99% or even 100% sequence identity at the amino acid level with any of SEQ ID NO: 1 to 17 or 19 to 33, preferably with SEQ ID NO: 1, in the production of one or more aroma compounds.

In addition, tagged enzyme versions of the alpha-ionylideneethane synthase having at least 50%, 55%, 60%, 65%, 66%, 70%, 71%, 75%, 76%, 80%, 81%, 85%, 86%, 90%, 91%, 92%, 93%, 94%, 95%, 96%, 97%, 98%, 99% or even 100% sequence identity at the amino acid level with any of SEQ ID NO: 1 to 17 or 19 to 33, preferably with SEQ ID NO: 1, may be used in the inventive methods for preparing alpha-ionone and/or alpha-ionylideneethane, the method comprising converting farnesyl diphosphate, into alpha-ionylideneethane, in the presence of an enzyme, the enzyme comprising a first segment comprising a tag peptide and a second segment comprising an alpha-ionylideneethane synthase, as described herein.

The tag-peptide is preferably selected from the group of nitrogen utilization proteins (NusA), thioredoxins (Trx), maltose-binding proteins (MBP), Glutathione S-transferases (GST), Small Ubiquitin-like Modifier (SUMO) or Calcium-binding proteins (Fh8), and functional homologues thereof. As used herein, a functional homologue of a tag peptide is a tag peptide having at least about the same effect on the solubility of the tagged enzyme, compared to the non-tagged enzyme. Typically, the homologue differs in that one or more amino acids have been inserted, substituted, deleted from, or extended to the peptide of which it is a homologue. The homologue may in particular comprise one or more substitutions of a hydrophilic amino acid for another hydrophilic amino acid, or of a hydrophobic amino acid for another. The homologue may, in particular, have a sequence identity of at least 40%, more in particular of at least 50%, preferably of at least 55%, more preferably of at least 60%, at least 70%, at least 75%, at least 80%, at least 85%, at least 90%, at least 95%, at least 98% or at least 99% sequence identity with the sequence of a NusA, Trx, MBP, GST, SUMO or Fh8.

Particularly suitable is the maltose-binding protein from Escherichia coli, or a functional homologue thereof.

The use of a tagged enzyme according to the invention is in particular advantageous in that it may contribute to an increased production, especially increased cellular production of alpha-ionylideneethane and/or alpha-ionone.

For improved solubility of the tagged enzyme (compared to the enzyme without the tag), the first segment of the enzyme is preferably bound at its C-terminus to the N-terminus of the second segment. Alternatively, the first segment of the tagged enzyme is bound at its N-terminus to the C-terminus of the second segment.

Further, the present invention is directed to an enzyme, comprising a first segment comprising a tag-peptide and a second segment comprising a polypeptide having enzymatic activity for converting a farnesyl diphosphate into alpha-ionylideneethane, in particular an alpha-ionylideneethane synthase, the tag-peptide preferably being selected from the group of MBP, NusA, Trx or SET, as well as nucleic acids encoding these and host cells harbouring said nucleic acids and producing said tagged enzymes.

Finally, the present invention pertains to the use of
a) the host cell of the invention, for
   (i) producing alpha-ionylideneethane, preferably E,Z-alpha-ionylideneethane (1,5,5-trimethyl-6-[(1E,3Z)-3-methyl-penta-1,3-dienyl]cyclohexene), preferably as an aroma ingredient, a precursor of an aroma substance, or as a precursor of vitamin A;
   (ii) producing alpha-ionone, preferably R-alpha-ionone;
   (iii) producing vitamin A;
   (iv) converting alpha-ionylideneethane to alpha-ionone;
   (v) converting alpha-ionylideneethane to vitamin A;
   (vi) for heterologous reconstitution of a terpene or terpenoid;
   (vii) for producing an industrial product, preferably an aroma composition, flavour or fragrance, a pharmaceutical composition, an agricultural composition, animal feed, a human nutritional product, a cosmetic, a colorant (carotenoid) or a radical scavenger;
   (viii) a fermentative production system for producing a sesquiterpene, preferably in a host cell as defined herein, i.e. a bacterial cell, a yeast cell, a fungal cell, an algal cell or a cyanobacterial cell, a non-human animal cell or a non-human mammalian cell, or a plant cell, more preferably a bacterial cell, or a yeast cell;
b) alpha-ionylideneethane as an aroma chemical or compound.

### EMBODIMENTS

1. Producing alpha-ionylideneethane, preferably E,Z-alpha-ionylideneethane (1,5,5-trimethyl-6-[(1E,3Z)-3-methyl-penta-1,3-dienyl]cyclohexene) and/or alpha-ionone by the methods of the invention.

2. Aroma chemical composition comprising the compound of embodiment 1 and:
(i) at least one additional aroma chemical different from alpha-ionylideneethane or alpha-ionone, or
(ii) at least one non-aroma chemical carrier, or
(iii) a mixture of (i) and (ii).

3. The composition according to embodiment 2, wherein the at least one aroma chemical different from alpha-ionylideneethane or alpha-ionone is selected from the group consisting of geranyl acetate, alpha-hexylcinnamaldehyde, 2 phenoxyethyl isobutyrate, dihydromyrcenol, methyl dihydrojasmonate, 4,6,6,7,8,8 hexamethyl-1,3,4,6,7,8-hexahydrocyclopenta[g]benzopyran, tetrahydrolinalool, ethyllinalool, benzyl salicylate, 2 methyl-3-(4-tert-butylphenyl)propanal, cinnamyl alcohol, 4,7 methano-3a,4,5,6,7,7a-hexahydro-5 indenyl acetate and/or 4,7 methano-3a,4,5,6,7,7a-hexahydro-6-indenyl acetate, citronellol, citronellyl acetate, tetrahydrogeraniol, vanillin, linalyl acetate, styrolyl acetate, octahydro-2,3,8,8-tetramethyl-2-acetonaphthone and/or 2-acetyl-1,2,3,4,6,7,8-octahydro-2,3,8,8-tetramethylnaphthalene, hexyl salicylate, 4-tert-butylcyclohexyl acetate, 2-tert-butylcyclohexyl acetate, alpha-ionone, alpha-methylionone, alpha-isomethylionone, coumarin, terpinyl acetate, 2-phenylethyl alcohol, 4-(4-hydroxy-4-methylpentyl)-3-cyclohexene-carboxaldehyde, alpha-amylcinnamaldehyde, ethylene brassylate, (E) and/or (Z)-3-methylcyclopentadec-5 enone, 15-pentadec-11-enolide and/or 15-pentadec-12-enolide, 15-cyclopentadecanolide, 1-(5,6,7,8-tetrahydro-3,5,5,6,8,8-hexamethyl-2-naphthalenyl)ethanone, 2-isobutyl-4-methyltetrahydro-2H pyran-4-ol, 2-ethyl-4-(2,2,3-trimethyl-3-cyclopenten-1-yl)-2-buten-1-ol, cis-3-hexenyl acetate, trans-3-hexenyl acetate, trans-2/cis-6-nonadienol, 2,4-dimethyl-3-cyclohexene carboxaldehyde, 2,4,4,7-tetramethyloct-6-en-3-one, 2,6-dimethyl-5-hepten-1-al, borneol, 3-(3-isopropylphenyl)butanal, 2-methyl-3-(3,4-methylenedioxyphenyl) propanal, 3-(4-ethyl-phenyl)-2,2-dimethylpropanal, 7-methyl-2H 1,5-benzodioxepin-3(4H)-one, 3,3,5-trimethyl-cyclohexyl acetate, 2,5,5 trimethyl-1,2,3,4,4a,5,6,7-octahydronaphthalen-2-ol, 3-(4-tert-butylphenyl)-propanal, ethyl 2-methylpentanoate, ethoxymethoxycyclododecane, 2,4-dimethyl-4,4a,5,9b-tetrahydroindeno[1,2-d][1,3]dioxine, (2-tert-butylcyclohexyl) acetate and 3-[5,5,6-trimethylbicyclo[2.2.1]hept-2-yl]cyclohexan-1-ol.

4. The composition according to embodiment 2 or 3, wherein the at least one non-aroma chemical carrier (ii) is selected from the group consisting of surfactants, oil components, anti-oxidants, deodorant-active agents and solvents.

5. The composition according to embodiment 4, wherein the solvent is selected from the group consisting of ethanol, isopropanol, diethylene glycol monoethyl ether, glycerol, propylene glycol, 1,2-butylene glycol, dipropylene glycol, triethyl citrate and isopropyl myristate.

6. The composition according to embodiment 5, wherein the at least one solvent is present in the composition in amount of 0.01 wt.-% to 99.0 wt.-%, based on the total weight of the composition.

7. The composition according to embodiment 5, wherein the at least one deodorant-active agent is selected from the group consisting of anti-perspirants, esterase inhibitors and antibacterial agents.

8. The composition according to embodiment 5, wherein the at least one surfactant is selected from the group consisting of anionic, non-ionic, cationic, amphoteric and zwitterionic surfactants.

9. The aroma chemical composition according to any one of embodiments 2 to 8 which is an aromatized ready-to-use composition.

10. The aroma chemical composition according to embodiment 9, wherein the aromatized ready-to-use composition is selected from perfume compositions, body care compositions, hygiene articles, cleaning compositions, textile detergent compositions, compositions for scent dispensers, foods, food supplements, pharmaceutical compositions and crop protection compositions.

11. Use of the compound according to embodiment 1 as an aroma chemical.

12. Use of the compound according to embodiment 1 for preparing an aroma chemical composition.

13. Use of the compound according to embodiment 1 for modifying the aroma character of an aroma chemical composition.

14. The use according to embodiments 11 to 13, wherein the aroma chemical composition is an aromatized ready-to-use composition.

15. The use according to embodiment 14, wherein the aromatized ready-to-use composition is selected from perfume compositions, body care compositions, hygiene articles, cleaning compositions, textile detergent compositions, compositions for scent dispensers, foods, food supplements, pharmaceutical compositions and crop protection compositions.

### Sequences

SEQ ID NO. 1 to 17 and 19 correspond to the amino acid sequences of alpha-ionylideneethane synthases shown in Table 1.

SEQ ID NO. 18 corresponds to Rhodobacter codon-optimized DNA encoding the amino acid sequence of SEQ ID NO. 1.

SEQ ID NO: 20 to 33 correspond to synthetic alpha-ionylideneethane synthases inventively created by the inventors.

### FIGURES

FIG. 1 E,Z-alpha-ionylideneethane (1,5,5-trimethyl-6-[(1E,3Z)-3-methyl-penta-1,3-dienyl]cyclohexene; E,Z-IE, 1) is the first cyclic intermediate of fungal abscisic acid (2) biosynthesis. It is formed by an alpha-ionylideneethane synthase (IE synthase) from farnesyl pyrophosphate (3).

FIG. 2 There are reports in literature that claim alpha-ionylideneethane synthases which, contrary to the enzyme used in the following Examples, cyclize farnesyl diphosphate to the cyclohexenepentadienol derivative 4; see Okamoto et al., Phytochemistry, Volume 27, Issue 11, 1988, Pages 3465-3469).

FIG. 3 shows GC traces of t-BME extracts from Rhodobacter ROB034 from DASGIP-fermenters (A) and shake flask cultivation (B), respectively. The peak with a retention time of 6.4 min was identified as alpha-ionone.

FIG. 4 shows alpha-ionone (4)=(E)-4-((2,6,6-trimethylcyclohex-2-en-1-yl)but-3-en-2-one.

FIG. 5 illustrates that R-alpha-ionone (R-4) is probably formed by oxidative cleavage of alpha-ionylideneethane (1).

FIG. 6 illustrates a method for preparing vitamin A, encompassing conversion of alpha-ionylideneethane via the respective alcohol to (2E,4E)-3-methyl-5-(2,6,6-trimethylcyclohex-2-en-1-yl)penta-2,4-dien-1-ol, followed by Wittig salt formation and reaction with C5-aldehyde.

FIG. 7 shows an alignment of the alpha-ionylideneethane synthase of SEQ ID NO: 1 and other alpha-ionylideneethane synthases. Conserved amino acids are shown by white font on black background.

The invention will now be illustrated by the following examples which shall, however, not be construed as limiting the scope of the present invention.

### EXAMPLES

### Summary

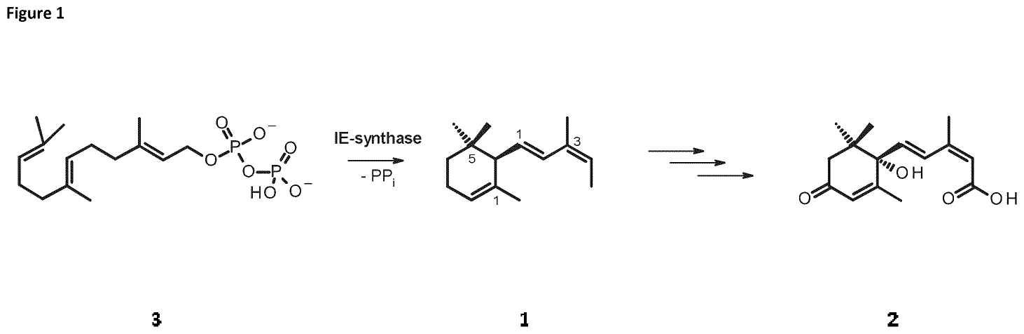

E,Z-alpha-ionylideneethane (1,5,5-trimethyl-6-[(1E,3Z)-3-methyl-penta-1,3-dienyl]cyclohexene; E,Z-IE, 1) is the first cyclic intermediate of fungal abscisic acid (2) biosynthesis. It is formed by a specific sesquiterpene synthase from farnesyl pyrophosphate (3); see FIG. 1.

An alpha-ionylideneethane synthase (IES) from Botrytis cinerea (SEQ ID NO. 1) was successfully cloned and expressed in Rhodobacter sphaeroides in order to assess the production of 1 as potential precursor for vitamin A.

After scaling the production of 1 from shake flasks to DASGIP-laboratory fermenters (approximately 11 working volume), a novel compound was detected in the dodecane phase of the fermentation broth, i.e. alpha-ionone. The isolation and identification of this compound is summarised in the following.

### Example 1: Expression of the Gene for the Alpha-Ionylideneethane Synthase (IES) from Botrytis cinerea in Rhodobacter

### 1.1 Construction of the Production System

The DNA sequence of the alpha-ionylideneethane synthase is from transcript Bcin08g03880.1 of Botrytis cinerea B05.10 (ASM83294v1). The respective gene (Bcin08g03880) is located at position 1,491,127-1,494,679 on chromosome 8. The data were extracted from the Ensembl Fungi release database (Ensembl Genomes 2020-enabling non-vertebrate genomic research, Nucleic Acids Research, 2019, [doi.org/10.1093/nar/gkz890]) and were used as template for the custom synthesis of an alpha-ionylideneethane synthase gene with a codon usage adapted to Rhodobacter sphaeroides (BioCat, Heidelberg) (SEQ ID NO. 18). The alpha-ionylideneethane synthase gene was cloned into the location of the santalene synthase gene in the plasmid p-m-SPppa-MBP-CiCaSSy-mpmii alt, known from WO2018160066. The newly created plasmid was designated as pROB018. Like the santalene synthase in the template plasmid, the alpha-ionylideneethane synthase protein will be produced as an N-terminal fusion to the maltose binding protein from E. coli. Furthermore, the plasmid contains all genes for the mevalonate pathway which ultimately delivers farnesyl diphosphate as substrate for the alpha-ionylideneethane synthase. In addition to this, Rhodobacter also contains the deoxyxylulose phosphate (DXP) pathway, as supplementary source of farnesyl diphosphate on its chromosome.

Transfer of the plasmid to Rhodobacter was done using standard procedures (see, for example, US260709B2, WO2014014339 and WO2011074954). The plasmid was transformed in E. coli S 17 and then transferred to Rhodobacter ROB002 by conjugation. Cultivation on a malic acid medium eliminates contamination by E. coli. Absence of contamination by E. coli was shown by PCR-amplification using E. coli-lacZ-specific oligonucleotides known in the art.

### 1.2 Cultivation of ROB034

Rhodobacter ROB034 harbouring the alpha-ionylideneethane synthase gene from Botrytis cinerea on the plasmid pROB018 was cultivated according to known methods, such as described in WO2018160066, in the DASGIP system.

Preculture 250 ml mROB002 medium in a 11 unbaffled Erlenmeyer-flask was inoculated with 1.5 ml cryo-stock culture. After incubation at 30° C. for 26 h (250 rpm, 5 cm amplitude), 69 ml preculture medium was used to inoculate the main culture.

Main culture started with 0.6 l mROB001 medium plus 10% (w/w) dodecane and was fed with a total of 646 ml feed solution according to standard procedures. After 141 h, the fermentation was terminated.

### Example 2: Isolation of Terpenes

### 2.1 Work-Up

1225 g fermentation broth was extracted with 800 g t-BME by stirring for 30 min. Since no obvious phase separation was observed, 25 ml DMSO and 100 g NaCl were added. Further improvement of phase separation was achieved by centrifugation at 5000*g for 15 min. The organic layer (653 g) was the decanted, the aqueous layer (1301 g) was discarded. The clear organic layer was dried with Na₂SO₄ and concentrated by rotary evaporation.

### 2.2 Purification of Alpha-Ionylldeneethane

From 89.6 g crude reaction extract (35 GC-a % IE, 2.6 GC-a % alpha-ionone, 57 GC-a % dodecane) dodecane was removed by distillation (250 mL distillation apparatus with distillation bridge): T_{bath}=up to 128° C., Tᵢₙ=92-103° C., T_{dist}=89-94° C., p=7-10 mbar.

The sump resulting from dodecane removal was further distilled using the “Pilot-Dist Spaltrohrkolonne” (M311 L4-06) at 2 mbar and T_{head}=80° C. Further purification of distillation fractions was performed by column chromatography (cyclohexane:ethyl acetate).

### 2.3 Purification of Alpha-Ionone

Dodecane was removed by distillation using the split tube distillation column” (at 30 mbar and Thead=106° C.) from 49.8 g crude reaction mixture (5.2 GC-a % alpha-ionone, 1.7 GC-a % alpha-ionylideneethane, 81 GC-a % dodecane); alpha-ionylideneethane as well as alpha-ionone evaporated already at 2 mbar and Thead=˜81° C.).

### 2.4 Analytics

### GC Analytics

Preparation of GC-Samples from Fermentation Broth

NaCl is added to the sample to improve phase separation. The sample is mixed on a vortex shaker until all salt has dissolved. Solid matter (i.e. biomass) is removed by centrifugation (20 min, 15° C., 4500*g) and the top liquid dodecane layer is removed.

100 μl dodecane are mixed with 900 μl acetone-internal standard solution and the sample is analysed by GC (methods: GC107B_0672_b-Bisabolene, A030_GC107B_0672

Isobionics_qual, column: Optima35 MS, 30 m*0.25 mm*0.25 μm) or by RCS/ON—M311 (method: GC610, CP-SIL 50 m; 0.32 mm ID; 1.2 μm FD; 80° C.—8 min; −250° C.—34 min; T injection=250° C., T detection=280° C.).

### GC-MS and NMR

For analysis of the broth, GC-MS and NMR were done.

### Polarimetry

The specific rotation was determined on a Jasco P2000 polarimeter equipped with a sodium-vapor lamp and a 1 dm-quartz cuvette. Samples were dissolved in chloroform and measured at room temperature.

### 2.5 Results

### 2.5.1 Identification of Alpha-Ionone

When the recombinant Rhodobacter expressing the B. cinerea alpha-ionylideneethane synthase was grown in a DASGIP-fermenter, the formation of an additional compound was observed in gas chromatograms of the fermentation broth. This substance is hardly detectable when growing the strain in shake flask cultures.

TABLE GC

| | α- | | |
|---|---|---|---|
| | ionylideneethane | | α- |
| Compound | (1) | Farnesene | ionone (4) |
| Approx. Time (min) | 5.3 | 5.9 | 6.5 |
| Response [pA]* | 65/300 | 10 or less | 75/10 to 20 |

*response for samples from DASGIP-fermenters with sizeable production of alpha-ionylideneethane (1) and alpha-ionone (4) simultaneously/response for samples from shake flasks with alpha-ionylideneethane (1) overproduction

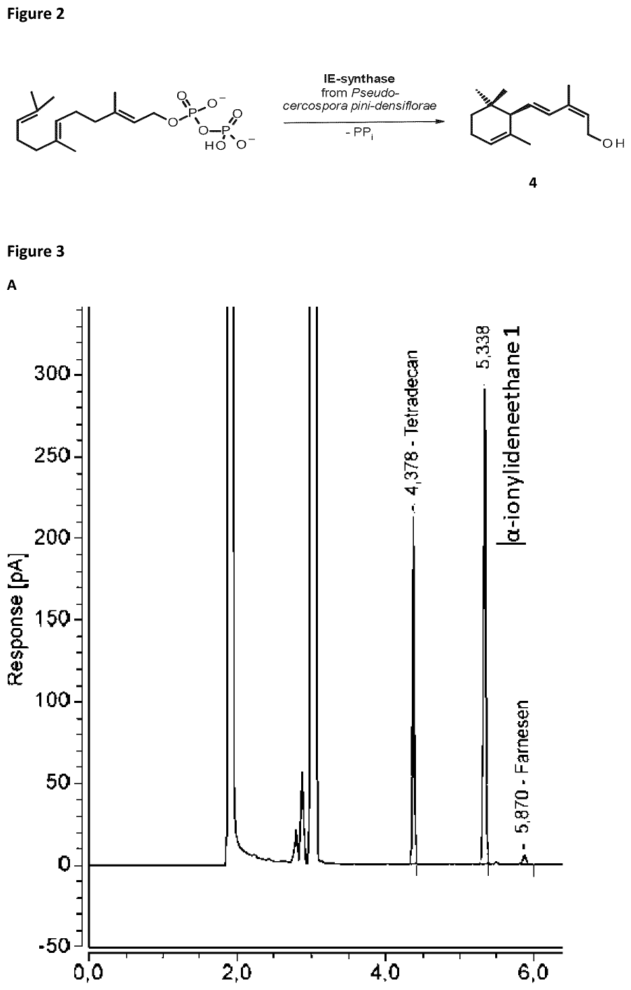

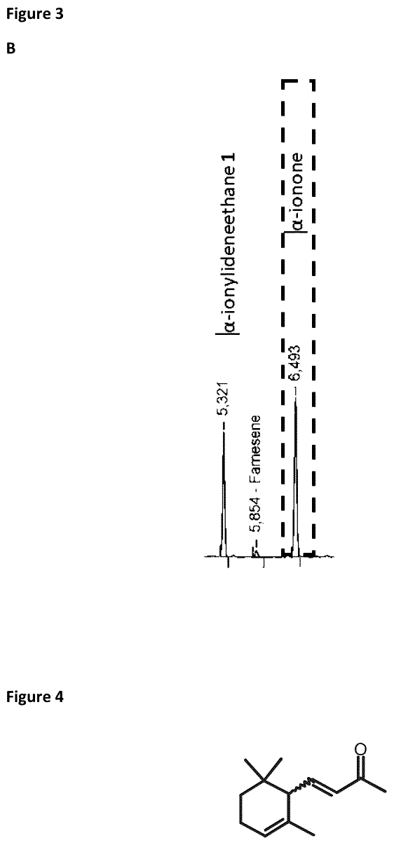

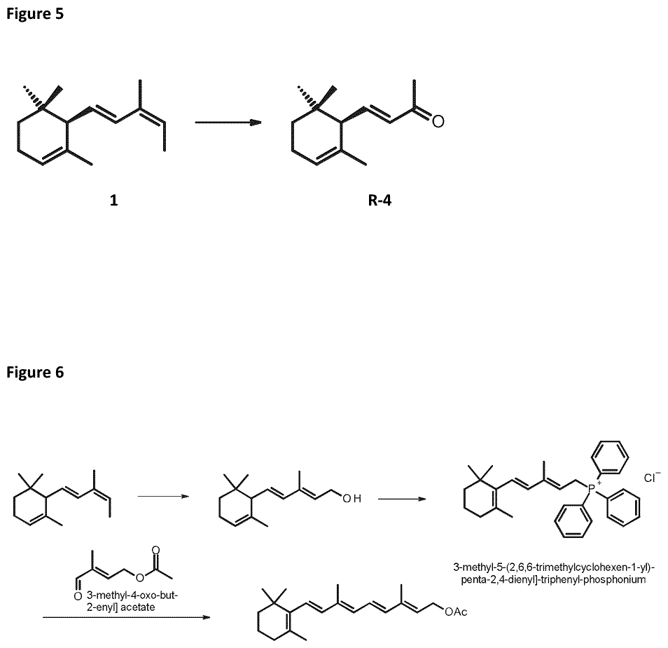

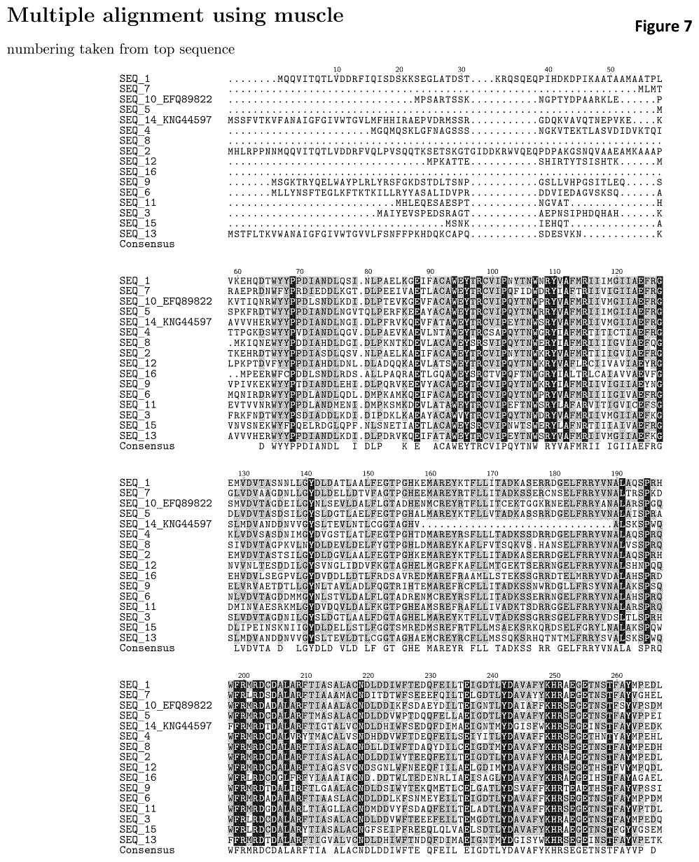

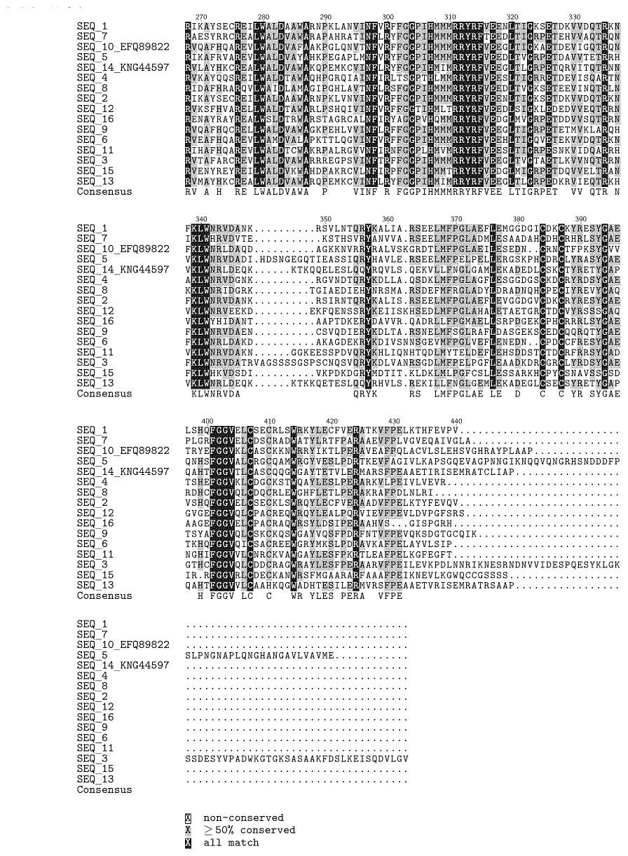

The novel peak was analysed by GC-MS which showed a mass of 192 g/mol. Interpretation of the mass spectra suggested that this compound is alpha-ionone (4-(2,6,6-trimethylcyclohex-2-en-1-yl)but-3-en-2-one) or its isomer (2,6,6-trimethylcyclohex-2-en-1-ylidene)butan-2-one). GC-analysis with authentic alpha-ionone showed identical retention time of the novel compound. FIG. 2 shows the formula of alpha-ionone (4)=(E)-4-((2,6,6-trimethylcyclohex-2-en-1-yl)but-3-en-2-one.

2.5.2 Purification of Terpenes from Fermentation Broth

For corroboration of the molecule's structure and further characterization, terpenes were isolated from the fermentation broth.

### Alpha-Ionylideneethane

Work-up of the total fermentation broth by tBME-extraction yielded 89.6 g of a clear, dark-brown solution. The crude reaction mixture was purified by distillation.

The obtained distillation sump (32 g) contained 70 GC-a % alpha-ionylideneethane and 7 GC-a % of dodecane. Loss of alpha-ionylideneethane (12 and 28 GC-a % in distillate 1 and 2) occurred within the two distillates taken. Based on GC-a % it accounts for a loss of ˜30% alpha-ionylideneethane which has to be optimized either by distillation conditions and/or by another second phase during fermentation: rather than dodecane a high-boiling solvent as co-solvent should be used since it would be preferred to evaporate the terpene products rather than the cosolvent (i.e. dodecane).

DMSO was added in the first extraction step to facilitate phase separation.

The subsequent “Pilot-Dist” sump distillation gave in total 7.5 g (6 fractions) with purities of 87-89 GC-a % alpha-ionylideneethane. The major by-product was alpha-ionone (8-9 GC-a %).

The following alpha-ionylideneethane samples were obtained by a final purification step using column chromatography with cyclohexane:ethyl acetate as eluent:

| | | | | NMR | |
|---|---|---|---|---|---|
| | | GC-a % | | [mol %] | |
| Identifier | [g] | E,Z-•-IE | all E-•IE | or ratio | Comment |
| 17-29 | 6.31 | 97 | 2 | >95 | Intended to |
| | | | | | assess allylic |
| | | | | | oxidation of IE |

### Alpha-Ionone

Distillation of 49.8 g crude reaction extract resulted in 6.8 g sump (55.4 GC-a % of alpha-ionone). After purification by column chromatography (cyclohexane:ethyl acetate). Fractions BOH-L-42 Fr. 36-42 were further purified in a second column chromatography (cyclohexane:ethyl acetate) to give BOH-L-47 Fr. 34-42. Similarly, BOH-L-52 Fr. 42-56 were purified from another fermentation run.

The following alpha-ionone fractions were obtained:

| | | | NMR | |
|---|---|---|---|---|
| Identifier | [g] | GC-a % | [mol %] | ee [%] |
| 36-42 | 1.71 | 91 | 85-90 | 99.54 |
| 34-42 | 0.82 | 98 | >95 | n.d. |
| 42-56 | 0.86 | 98 | n.d. | n.d. |

On aggregate two purification experiments were performed in this study: after removing tBME and in a subsequent step—dodecane from crude fermentation product by distillation, column chromatography was applied to yield
a) 6.3 g alpha-ionylideneethane sump in a purity of 97 GC-a % 0.09 GC-a % of alpha-ionone) from 32 g distillation sump as well as
b) 0.82 g alpha-ionone that could be isolated in a purity of 98 GC-a % (different by-products) from 6.8 g sump.

¹³C-NMR confirmed the following structures:

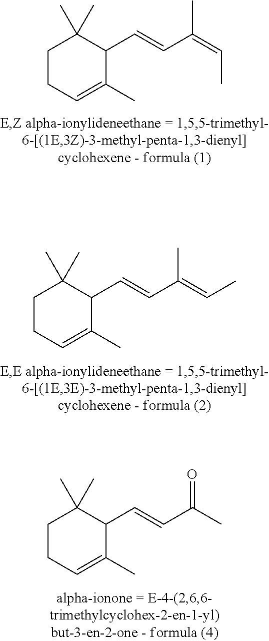

### 2.5.3 Stereochemistry of Isolated Terpenes

Also, it could be shown that alpha-ionone isolated from the fermentation broth is almost optically pure: this material gave only a single peak on a chiral GC with the same retention time as one of two peaks from the racemic standard.

Alpha-ionone isolated from fermentation broth was additionally analysed by polarimetry to give a specific rotation of +388° [α]_{D}²⁰ (c 0.75, CHCl₃). This value is in fair accordance with literature data for the R-enantiomer.

Similarly, alpha-ionylideneethane yielded a specific rotation of +441° [α]_{D} (c 0.762, CHCl₃).

### 2.5.4 Olfactory Notes

### Olfactory Assessment:

A 1% weight solution of alpha-ionylideneethane as obtained in Example 2.5.2 in triethylcitrate was prepared and evaluated by a panel of four professional perfumers at room temperature at about 20° C. using freshly dipped blotter paper. The olfactory notes were ranked from 1 (very weak) to 9 (strong).

TABLE
Olfactory assessment

| | Floral- | Woody-Orris |
|---|---|---|
| Olfactory note | Violet | (Iris) Root |
| alpha-ionylideneethane | 9 | 9 |

### Advantageous Perfume Components

Alpha-ionylideneethane or alpha-ionone is formulated in the perfume compositions according to the following two Tables; compound A is to be understood to be alpha-ionylideneethane or alpha-ionone.

TABLE
Fragrance compositions 1A and 1B

| | 1A | 1B |
|---|---|---|
| Lactone C10 gamma (5-hexyloxolan-2-one) | 2 | 2 |
| Bourgeonal (3-(4-tert-butylphenyl)propanal) | 2 | 2 |
| Citronellol | 3 | 3 |
| Aldehyde C-14 (5-heptyloxolan-2-one) | 3 | 3 |
| Allyl heptylate | 4 | 4 |
| Amber core (1-(2-tert-butylcyclohexyl) | 4 | 4 |
| oxybutan-2-ol) | | |
| Ethyl-2-methyl butyrate | 4 | 4 |
| Geranyl acetate | 5 | 5 |
| Helional (3-(1,3-benzodioxol-5-yl)-2- | 10 | 10 |
| methylpropanal) | | |
| Manzanate (ethyl 2-methylpentanoate) | 10 | 10 |
| Amberwood (ethoxymethoxycyclododecane) | 10 | 10 |
| Hexyl acetate | 11 | 11 |
| Benzyl salicylate | 12 | 12 |
| Magnolan (2,4-dimethyl-4,4a,5,9b- | 15 | 15 |
| tetrahydroindeno[1,2-d][1,3]dioxine) | | |
| Verdox (2-tert-butylcyclohexyl) acetate) | 25 | 25 |
| Bergamot oil bergaptene free | 25 | 25 |
| Linalol | 30 | 30 |
| Dipropylene glycol | 45 | 45 |
| Iso E Super (Tetramethyl | 110 | 110 |
| acetyloctahydronaphthalenes) | | |
| Pyranol (4-methyl-2-(2-methylpropyl)oxan- | 170 | 170 |
| 4-ol) | | |
| Hedione (methyl 3-oxo-2- | 200 | 200 |
| pentylcyclopentaneacetate) | | |
| Galaxolide 50% IPM (1,3,4,6,7,8-hexahydro- | 300 | 300 |
| 4,6,6,7,8,8-hexamethylcyclopenta(g)-2- | | |
| benzopyran 50% in isopropyl myristate) | | |
| Compound A | 5 | 20 |
| | 1005 | 1020 |

TABLE
Fragrance compositions 2A and 2B

| | 2A | 2B |
|---|---|---|
| Raspberry ketone (4-(4-hydroxyphenyl)butan-2-one) | 4 | 4 |
| Vanitrope (2-ethoxy-5-prop-1-enylphenol) | 6 | 6 |
| Cyclamen aldehyde (at least 90% 2-methyl- | 10 | 10 |
| 3-(p-isopropylphenyl)propionaldehyde; | | |
| secondary component: 5% 3-(p-cumenyl)-2- | | |
| methylpropionic acid) | | |
| Bicyclononalactone (3,4,4a,5,6,7,8,8a- | 10 | 10 |
| octahydrochromen-2-one) | | |
| Aldehyde C-14 (5-heptyloxolan-2-one) | 14 | 14 |
| Ethylvanillin (3-ethoxy-4-hydroxybenzaldehyde) | 16 | 16 |
| Heliotropine (1,3-benzodioxole-5-carbaldehyde) | 20 | 20 |
| Iso E Super (tetramethyl acetyloctahydronaphthalenes) | 20 | 20 |
| Sandela (3-[5,5,6-trimethylbicyclo[2.2.1]hept- | 30 | 30 |
| 2-yl]cyclohexan-1-ol) | | |
| Vanillin isobutyrate ((4-formyl-2-methoxyphenyl) | 40 | 40 |
| 2-methylpropanoate) | | |
| Aldehyde C-18 (5-pentyloxolan-2-one) | 50 | 50 |
| Benzyl salicylate | 60 | 60 |
| Hexyl cinnamic aldehyde (2-(phenylmethylidene)octanal) | 70 | 70 |
| Hedione (methyl 3-oxo-2-pentylcyclopentaneacetate) | 130 | 130 |
| Pyranol (4-methyl-2-(2-methylpropyl)oxan-4-ol) | 150 | 150 |
| Ethylene brassylate (1,4-dioxacycloheptadecane- | 170 | 170 |
| 5,17-dione) | | |
| Galaxolide 50% IPM (1,3,4,6,7,8-hexahydro- | 200 | 200 |
| 4,6,6,7,8,8-hexamethylcyclopenta(g)-2- | | |
| benzopyran 50% in isopropyl myristate) | | |
| Compound A | 5 | 20 |
| | 1005 | 1020 |

The examples of olfactory notes of Table: Olfactory assessment, and the fragrance composition according to Table: Fragrance compositions 1A and 1B. and according to Table: Fragrance compositions 2A and 2B, namely 1A, 1B, 2A, 2B, could be included in various compositions enlisted below:
Deo pump spray
Clean hair conditioner
Face wash gel
Foam bath concentrate
Hair gel
Self-foaming bodywash
Sprayable sun care emulsion
Sprayable sun protection emulsion
Emollient facial gel
2-phases oil foam bath
Shampoos
Shower bath
Hydro-alcoholic AP/Deo pump spray
Aerosol
Aqueous/alcoholic AP/Deo roll-on
Styling Gel Type “Out of Bed”
Shaving Foam
Sensitive skin Baby shampoo
Body wash for Sensitive Skin
Gloss Enhancing Shampoo for Sensitive Scalp
Deo Stick
Baby Wipe
After shave balm
Face Gel
Face Day Care Cream
Face Cleanser
Body lotion
Sun Care SPF50+, Sprayable Lotion
Hand dish cleaner, regular
Hand dish cleaner, concentrate
Sanitary cleaner, concentrate
All-purpose cleaner
Anti-bacterial fabric softener
Detergent composition
Powder detergent composition
Liquid detergent composition

A person skilled in art may be well versed with the various general formulations for the above-mentioned products.

Perfume oil compositions 1A, 1B, 2A and 2B can be, for example, formulated in specific formulations as disclosed in, IP.com Number: IPCOM000258614D entitled New Aroma Chemicals pages 6 to 46, Table 1 to Table D13, wherein the “Fragrance Composition 1A” is replaced by identical amounts of perfume oil compositions 1A, 1B, 2A or 2B.

1.-15. (canceled)

16. An aroma compound comprising alpha-ionylideneethane.

17. The aroma compound of claim 16, wherein the aroma compound has a note of Floral-Violet and/or Woody-Orris/Iris Root.

18. The aroma compound of claim 16, wherein alpha-ionylideneethane is produced by an alpha-ionylideneethane synthase.

19. The aroma compound of claim 18, wherein the alpha-ionylideneethane synthase is selected from the group consisting of:
a) the alpha-ionylideneethane synthase belongs to the subclass of carbon-oxygen lyases acting on phosphates (EC 4.2.3);
b) the alpha-ionylideneethane synthase is a fungal or bacterial alpha-ionylideneethane synthase; and
c) the alpha-ionylideneethane synthase comprises an amino acid sequence selected from the group consisting of:
i) an amino acid sequence as shown in any of SEQ ID NOs. 1, 2, 3, 4, 5, 6, 7, 8, 9, 10, 11, 12, 13, 14, 15, 16, 17, 19, 20, 21, 22, 23, 24, 25, 26, 27, 28, 29, 30, 31, 32, or 33;
ii) an amino acid sequence having at least 40% sequence identity at the amino acid level with any of SEQ ID NOs. 1, 2, 3, 4, 5, 6, 7, 8, 9, 10, 11, 12, 13, 14, 15, 16, 17, 19, 20, 21, 22, 23, 24, 25, 26, 27, 28, 29, 30, 31, 32, or 33, having alpha-ionylideneethane synthase activity; and
iii) an enzymatically active fragment of the amino acid sequence of a) or b), having alpha-ionylideneethane synthase activity; and
d) any combination of a) to c) above.

20. A method for producing one or more aroma compounds comprising utilizing the ionylideneethane synthase of claim 19.

21. A method for preparing one or more aroma compounds, comprising
a) providing farnesyl diphosphate and an alpha-ionylideneethane synthase, under conditions suitable for the alpha-ionylideneethane synthase to produce alpha-ionylideneethane,
b) converting farnesyl diphosphate to alpha-ionylideneethane, in vitro or in a host cell,
c) optionally, converting alpha-ionylideneethane to one or more further aroma compounds,
d) isolating alpha-ionylideneethane and/or the optionally one or more further aroma compounds and,
e) optionally, purifying alpha-ionylideneethane and/or the optionally one or more further aroma compounds.

22. The method of claim 21, wherein the method includes the further steps of:
f) exposing alpha-ionylideneethane to conditions suitable for oxidative cleavage of alpha-ionylideneethane to produce alpha-ionone, and
g) converting alpha-ionylideneethane to alpha-ionone; and
h) optionally, purifying the alpha-ionone.

23. The method of claim 21, wherein the alpha-ionylideneethane synthase is selected from the group consisting of:
a) the subclass of carbon-oxygen lyases acting on phosphates (EC 4.2.3);
b) a fungal or bacterial alpha-ionylideneethane synthase; and
c) an amino acid sequence selected from the group consisting of:
i) an amino acid sequence as shown in any of SEQ ID NOs. 1, 2, 3, 4, 5, 6, 7, 8, 9, 10, 11, 12, 13, 14, 15, 16, 17, 19, 20, 21, 22, 23, 24, 25, 26, 27, 28, 29, 30, 31, 32, or 33;
ii) an amino acid sequence having at least 40% sequence identity at the amino acid level with any of SEQ ID NO. 1, 2, 3, 4, 5, 6, 7, 8, 9, 10, 11, 12, 13, 14, 15, 16, 17, 19, 20, 21, 22, 23, 24, 25, 26, 27, 28, 29, 30, 31, 32, or 33, having alpha-ionylideneethane synthase activity;
iii) an enzymatically active fragment of the amino acid sequence of a) or b), having alpha-ionylideneethane synthase activity; and
d) any combination of a) to c) above.

24. A method for imparting and/or enhancing an odor or flavor of a product, wherein the method comprises:
a) providing farnesyl diphosphate and the alpha-ionylideneethane synthase of claim 19, under conditions suitable for the alpha-ionylideneethane synthase to produce alpha-ionylideneethane,
b) converting farnesyl diphosphate to alpha-ionylideneethane, in vitro or in a host cell,
c) optionally, converting alpha-ionylideneethane to one or more further aroma compounds,
d) isolating alpha-ionylideneethane and/or the optionally one or more further aroma compounds,
e) optionally, purifying alpha-ionylideneethane and/or the optionally one or more further aroma compounds, and
f) contacting the product with alpha-ionylideneethane and/or the optionally one or more further aroma compound, thereby imparting and/or enhancing the odor or flavor of the product.

25. The method of claim 24, wherein the method includes the further steps of:
i) exposing alpha-ionylideneethane to conditions suitable for oxidative cleavage of alpha-ionylideneethane to produce alpha-ionone, and
ii) converting alpha-ionylideneethane to alpha-ionone; and
iii) optionally, purifying the alpha-ionone.

26. A composition and/or fragrance composition and/or perfumed or fragranced product, comprising:
i) the aroma compound of claim 16;
ii) optionally, at least one further aroma compound different from i), and
iii) optionally, at least one diluent.

27. A perfumed or fragranced product comprising the aroma compound of claim 16.

28. The perfumed or fragranced product of claim 27, wherein the aroma compound comprises E,Z-alpha-ionylideneethane (1,5,5-trimethyl-6-[(1E,3Z)-3-methyl-penta-1,3-dienyl]cyclohexene).

29. A method for producing alpha-ionone (4-(2,6,6-trimethylcyclohex-2-en-1-yl)but-3-en-2-one), comprising the steps in the following order:
a) contacting farnesyl diphosphate with at least one alpha-ionylideneethane synthase as defined in claim 19, under conditions suitable to produce at least one alpha-ionylideneethane, thereby producing the at least one alpha-ionylideneethane;
b) exposing the at least one alpha-ionylideneethane produced in step a) to conditions suitable for oxidative cleavage of alpha-ionylideneethane to produce alpha-ionone; and
c) optionally, isolating the alpha-ionone produced in step b).

30. A host cell for producing alpha-ionone (4-(2,6,6-trimethylcyclohex-2-en-1-yl)but-3-en-2-one), wherein the host cell comprises farnesyl diphosphate and the alpha-ionylideneethane synthase of claim 19, wherein the host cell is a bacterial cell, a yeast cell, a fungal cell, an algal cell, a cyanobacterial cell, a non-human animal cell, a non-human mammalian cell, or a plant cell, and the host cell is suitable for oxidative cleavage of alpha-ionylideneethane to produce alpha-ionone.

31. The host cell of claim 30, wherein:
(i) alpha-ionylideneethane synthase converts farnesyl diphosphate to alpha-ionylideneethane; and/or
(ii) alpha-ionylideneethane is converted to alpha-ionone by oxidative cleavage chemically and/or enzymatically.

32. A host cell comprising farnesyl diphosphate and a heterologous nucleic acid encoding the alpha-ionylideneethane synthase of claim 19.

33. The host cell of claim 32, wherein the host cell:
(i) produces alpha-ionylideneethane, preferably 2Z,4E-alpha-ionylideneethane (1,5,5-trimethyl-6-[(1E,3Z)-3-methyl-penta-1,3-dienyl]cyclohexene);
(ii) produces alpha-ionone, preferably R-alpha-ionone;
(iii) produces vitamin A;
(iv) converts alpha-ionylideneethane to alpha-ionone;
(v) converts alpha-ionylideneethane to vitamin A;
(vi) heterologously reconstitutes of a terpene or terpenoid; or
(vii) produces an industrial product.
